SEQUENCE LISTING (76 sequences)

SEQ ID NO: 1 - DNA, 3684 bp, synthetic construct
  misc_feature 1..3684: note = Synthetic polynucleotide. Codon optimized LbCpf1-CO1
  source 1..3684: mol_type = other DNA
atgtccaagcttgagaagtttactaattgctatagcctgtctaagacccttcgctttaaggcgattcctgtgggcaagacgcaggaaaacatcgacaacaagcgactgctggtggaagacgagaagcgcgctgaggactataagggcgtaaagaagctgctcgatcggtactacctgagttttatcaacgatgtgttgcattccataaagttgaagaatcttaataactacatctccttgtttcggaaaaagaccaggaccgagaaggagaataaagagttggaaaatctggagatcaacttgcgtaaggagatcgcgaaggcgttcaagggtaatgagggttataagagtctctttaaaaaagatataattgaaacgattctacccgaatttctggatgataaggatgagattgccctcgtcaattcgttcaatggctttacaacagcgtttacaggtttcttcgataacagggaaaatatgtttagcgaggaggcaaagtcgacctctatcgcttttcgctgtataaacgaaaatttaactcgatatatctccaatatggatattttcgagaaagtcgatgcgatctttgataagcatgaagtccaggagattaaggaaaagattcttaattcagattatgatgtggaagattttttcgaaggtgagttctttaacttcgtgcttacgcaagaaggaatcgacgtttacaatgcaataattggtgggtttgttactgaatctggtgaaaagatcaaaggcctcaatgagtacattaacttgtacaatcagaagacgaagcagaagttaccaaaattcaagccgctgtacaagcaagtgttatctgacagggaatctttgtccttttacggtgaaggatacacttctgatgaagaggtgcttgaggtcttcaggaatacactgaacaagaattctgagatcttctcctcaattaagaaactcgaaaaacttttcaagaactttgatgaatacagctctgctggaattttcgtaaagaatggtcccgccataagcactatctcaaaggacattttcggtgagtggaatgttataagagataaatggaatgcagagtacgacgatatccatttgaagaaaaaggcggtagttaccgaaaagtacgaggatgacagaaggaaatcgttcaagaagattggctcattctccctggagcagcttcaggagtacgcggacgcggacctttctgttgttgaaaagctcaaggagatcatcatacaaaaggtagacgagatttataaggtctatgggagctcagagaaattgttcgacgccgatttcgttttggagaagtcactgaaaaagaacgacgctgtcgtcgctattatgaaagaccttttggattctgtcaagtcttttgagaactatattaaggcttttttcggtgagggtaaggagacgaaccgcgacgagtcattctacggagactttgtactcgcatatgacatactgctcaaagttgatcatatttatgacgcgatccgcaattacgttacacaaaaaccatactctaaagataaattcaagctgtatttccaaaacccgcaattcatggggggctgggataaggataaggaaaccgattatagggcgaccatattgcgctacgggagcaagtattacttagcgatcatggataaaaaatacgcaaagtgtttgcaaaagatagacaaggacgatgtcaatggcaattatgagaagattaactataagttgctgccaggacccaataagatgttgcccaaagtttttttctccaaaaaatggatggcttattataaccctagcgaggacatccagaaaatatacaaaaacggcacatttaagaagggggatatgttcaatcttaatgattgtcacaagctgatagactttttcaaggactcaatctctcggtatcccaagtggtcgaatgcgtacgattttaatttttctgagaccgaaaagtacaaggatattgcaggcttttatcgcgaagtggaggaacaaggatacaaggtttcattcgaatccgcctcaaaaaaggaggtcgacaaactcgtcgaagagggtaaactgtacatgttccaaatttacaataaagacttttcagacaaatcacacggaactcctaaccttcacacaatgtactttaaattgctgttcgatgaaaataatcacggtcaaattaggctgtcaggcggagctgagcttttcatgaggagggctagtctgaagaaagaggagctggtggtccatcctgcaaatagtcccatagctaataagaatcctgataaccctaagaaaaccaccactctctcctacgacgtttataaggataaacggttcagtgaagatcagtatgagttgcatattcccattgccataaataagtgccctaagaacatcttcaaaattaacacagaagtgagagttctcttgaaacacgatgataatccatatgtgattgggatagataggggagagcgtaacctcctttatattgtcgtggttgacggaaagggtaacatagtggagcaatacagcctcaatgaaattattaacaactttaatggtattagaataaagactgactatcatagtctcttggataaaaaagagaaggagaggttcgaagctaggcagaattggacgtctattgaaaatattaaagaactcaaagcagggtacattagccaagtcgttcacaagatatgcgagttggttgagaaatatgatgctgtcattgcactggaggatctcaatagcggtttcaaaaacagtcgtgttaaggtggagaagcaggtttaccagaaattcgagaagatgctgattgataagcttaactatatggtggacaaaaagtctaatccatgcgcgaccggtggcgcacttaagggctatcagatcacaaacaagttcgagtcgtttaagtccatgtcaacacagaacggtttcatcttctatatcccggcatggctgacctcaaaaattgatcctagcacggggttcgtaaacttacttaaaactaaatacacctcaattgctgattcaaaaaagtttatatcctcatttgaccgaattatgtatgtgcccgaggaggacctgttcgaattcgctctggactataagaacttttcaagaacagatgcggattatatcaagaagtggaaactttacagttatggtaaccgcattaggatattccggaaccccaaaaaaaataatgtctttgattgggaggaggtatgtctgacgtctgcttataaggagctatttaataagtacggcatcaattatcagcagggggacatccgcgcgcttctctgcgagcaatccgataaggctttctacagctccttcatggcattgatgagcctcatgctgcagatgagaaacagtatcacaggtagaacggacgtagacttcctaatttctccagtgaagaattcagatggcatcttctatgatagccgcaactatgaggcacaggagaacgccatcctgcccaaaaatgctgatgccaacggtgcgtataacattgctaggaaggtcctctgggccataggtcaattcaagaaagctgaagacgagaagctcgacaaggtaaaaattgccatatccaacaaggagtggctcgaatatgcacagacctctgtgaagcat

SEQ ID NO: 2 - protein, 1228 aa, unidentified
  source 1..1228: note = Lachnospiraceae bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 3 - DNA, 30 bp, Lycopersicon esculentum
  source 1..30: mol_type = unassigned DNA
ggatctaagaagagaagaattaaacaagat

SEQ ID NO: 4 - DNA, 3750 bp, synthetic construct
  misc_feature 1..3750: note = Synthetic polynucleotide.NLS-LbCpf1-CO1-NLS
  source 1..3750: mol_type = other DNA
atgggatctaagaagagaagaattaaacaagatatgtccaagcttgagaagtttactaattgctatagcctgtctaagacccttcgctttaaggcgattcctgtgggcaagacgcaggaaaacatcgacaacaagcgactgctggtggaagacgagaagcgcgctgaggactataagggcgtaaagaagctgctcgatcggtactacctgagttttatcaacgatgtgttgcattccataaagttgaagaatcttaataactacatctccttgtttcggaaaaagaccaggaccgagaaggagaataaagagttggaaaatctggagatcaacttgcgtaaggagatcgcgaaggcgttcaagggtaatgagggttataagagtctctttaaaaaagatataattgaaacgattctacccgaatttctggatgataaggatgagattgccctcgtcaattcgttcaatggctttacaacagcgtttacaggtttcttcgataacagggaaaatatgtttagcgaggaggcaaagtcgacctctatcgcttttcgctgtataaacgaaaatttaactcgatatatctccaatatggatattttcgagaaagtcgatgcgatctttgataagcatgaagtccaggagattaaggaaaagattcttaattcagattatgatgtggaagattttttcgaaggtgagttctttaacttcgtgcttacgcaagaaggaatcgacgtttacaatgcaataattggtgggtttgttactgaatctggtgaaaagatcaaaggcctcaatgagtacattaacttgtacaatcagaagacgaagcagaagttaccaaaattcaagccgctgtacaagcaagtgttatctgacagggaatctttgtccttttacggtgaaggatacacttctgatgaagaggtgcttgaggtcttcaggaatacactgaacaagaattctgagatcttctcctcaattaagaaactcgaaaaacttttcaagaactttgatgaatacagctctgctggaattttcgtaaagaatggtcccgccataagcactatctcaaaggacattttcggtgagtggaatgttataagagataaatggaatgcagagtacgacgatatccatttgaagaaaaaggcggtagttaccgaaaagtacgaggatgacagaaggaaatcgttcaagaagattggctcattctccctggagcagcttcaggagtacgcggacgcggacctttctgttgttgaaaagctcaaggagatcatcatacaaaaggtagacgagatttataaggtctatgggagctcagagaaattgttcgacgccgatttcgttttggagaagtcactgaaaaagaacgacgctgtcgtcgctattatgaaagaccttttggattctgtcaagtcttttgagaactatattaaggcttttttcggtgagggtaaggagacgaaccgcgacgagtcattctacggagactttgtactcgcatatgacatactgctcaaagttgatcatatttatgacgcgatccgcaattacgttacacaaaaaccatactctaaagataaattcaagctgtatttccaaaacccgcaattcatggggggctgggataaggataaggaaaccgattatagggcgaccatattgcgctacgggagcaagtattacttagcgatcatggataaaaaatacgcaaagtgtttgcaaaagatagacaaggacgatgtcaatggcaattatgagaagattaactataagttgctgccaggacccaataagatgttgcccaaagtttttttctccaaaaaatggatggcttattataaccctagcgaggacatccagaaaatatacaaaaacggcacatttaagaagggggatatgttcaatcttaatgattgtcacaagctgatagactttttcaaggactcaatctctcggtatcccaagtggtcgaatgcgtacgattttaatttttctgagaccgaaaagtacaaggatattgcaggcttttatcgcgaagtggaggaacaaggatacaaggtttcattcgaatccgcctcaaaaaaggaggtcgacaaactcgtcgaagagggtaaactgtacatgttccaaatttacaataaagacttttcagacaaatcacacggaactcctaaccttcacacaatgtactttaaattgctgttcgatgaaaataatcacggtcaaattaggctgtcaggcggagctgagcttttcatgaggagggctagtctgaagaaagaggagctggtggtccatcctgcaaatagtcccatagctaataagaatcctgataaccctaagaaaaccaccactctctcctacgacgtttataaggataaacggttcagtgaagatcagtatgagttgcatattcccattgccataaataagtgccctaagaacatcttcaaaattaacacagaagtgagagttctcttgaaacacgatgataatccatatgtgattgggatagataggggagagcgtaacctcctttatattgtcgtggttgacggaaagggtaacatagtggagcaatacagcctcaatgaaattattaacaactttaatggtattagaataaagactgactatcatagtctcttggataaaaaagagaaggagaggttcgaagctaggcagaattggacgtctattgaaaatattaaagaactcaaagcagggtacattagccaagtcgttcacaagatatgcgagttggttgagaaatatgatgctgtcattgcactggaggatctcaatagcggtttcaaaaacagtcgtgttaaggtggagaagcaggtttaccagaaattcgagaagatgctgattgataagcttaactatatggtggacaaaaagtctaatccatgcgcgaccggtggcgcacttaagggctatcagatcacaaacaagttcgagtcgtttaagtccatgtcaacacagaacggtttcatcttctatatcccggcatggctgacctcaaaaattgatcctagcacggggttcgtaaacttacttaaaactaaatacacctcaattgctgattcaaaaaagtttatatcctcatttgaccgaattatgtatgtgcccgaggaggacctgttcgaattcgctctggactataagaacttttcaagaacagatgcggattatatcaagaagtggaaactttacagttatggtaaccgcattaggatattccggaaccccaaaaaaaataatgtctttgattgggaggaggtatgtctgacgtctgcttataaggagctatttaataagtacggcatcaattatcagcagggggacatccgcgcgcttctctgcgagcaatccgataaggctttctacagctccttcatggcattgatgagcctcatgctgcagatgagaaacagtatcacaggtagaacggacgtagacttcctaatttctccagtgaagaattcagatggcatcttctatgatagccgcaactatgaggcacaggagaacgccatcctgcccaaaaatgctgatgccaacggtgcgtataacattgctaggaaggtcctctgggccataggtcaattcaagaaagctgaagacgagaagctcgacaaggtaaaaattgccatatccaacaaggagtggctcgaatatgcacagacctctgtgaagcatggatctaagaagagaagaattaaacaagattga

SEQ ID NO: 5 - DNA, 189 bp, Solanum tuberosum
  source 1..189: mol_type = unassigned DNA
gtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcag

SEQ ID NO: 6 - DNA, 3939 bp, synthetic construct
  misc_feature 1..3939: note = Synthetic polynucleotide.NLS- LbCpf15'-Intron-LbCpf13'-NLS
  source 1..3939: mol_type = other DNA
atgggatctaagaagagaagaattaaacaagatatgtccaagcttgagaagtttactaattgctatagcctgtctaagacccttcgctttaaggcgattcctgtgggcaagacgcaggaaaacatcgacaacaagcgactgctggtggaagacgagaagcgcgctgaggactataagggcgtaaagaagctgctcgatcggtactacctgagttttatcaacgatgtgttgcattccataaagttgaagaatcttaataactacatctccttgtttcggaaaaagaccaggaccgagaaggagaataaagagttggaaaatctggagatcaacttgcgtaaggagatcgcgaaggcgttcaagggtaatgagggttataagagtctctttaaaaaagatataattgaaacgattctacccgaatttctggatgataaggatgagattgccctcgtcaattcgttcaatggctttacaacagcgtttacaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtttcttcgataacagggaaaatatgtttagcgaggaggcaaagtcgacctctatcgcttttcgctgtataaacgaaaatttaactcgatatatctccaatatggatattttcgagaaagtcgatgcgatctttgataagcatgaagtccaggagattaaggaaaagattcttaattcagattatgatgtggaagattttttcgaaggtgagttctttaacttcgtgcttacgcaagaaggaatcgacgtttacaatgcaataattggtgggtttgttactgaatctggtgaaaagatcaaaggcctcaatgagtacattaacttgtacaatcagaagacgaagcagaagttaccaaaattcaagccgctgtacaagcaagtgttatctgacagggaatctttgtccttttacggtgaaggatacacttctgatgaagaggtgcttgaggtcttcaggaatacactgaacaagaattctgagatcttctcctcaattaagaaactcgaaaaacttttcaagaactttgatgaatacagctctgctggaattttcgtaaagaatggtcccgccataagcactatctcaaaggacattttcggtgagtggaatgttataagagataaatggaatgcagagtacgacgatatccatttgaagaaaaaggcggtagttaccgaaaagtacgaggatgacagaaggaaatcgttcaagaagattggctcattctccctggagcagcttcaggagtacgcggacgcggacctttctgttgttgaaaagctcaaggagatcatcatacaaaaggtagacgagatttataaggtctatgggagctcagagaaattgttcgacgccgatttcgttttggagaagtcactgaaaaagaacgacgctgtcgtcgctattatgaaagaccttttggattctgtcaagtcttttgagaactatattaaggcttttttcggtgagggtaaggagacgaaccgcgacgagtcattctacggagactttgtactcgcatatgacatactgctcaaagttgatcatatttatgacgcgatccgcaattacgttacacaaaaaccatactctaaagataaattcaagctgtatttccaaaacccgcaattcatggggggctgggataaggataaggaaaccgattatagggcgaccatattgcgctacgggagcaagtattacttagcgatcatggataaaaaatacgcaaagtgtttgcaaaagatagacaaggacgatgtcaatggcaattatgagaagattaactataagttgctgccaggacccaataagatgttgcccaaagtttttttctccaaaaaatggatggcttattataaccctagcgaggacatccagaaaatatacaaaaacggcacatttaagaagggggatatgttcaatcttaatgattgtcacaagctgatagactttttcaaggactcaatctctcggtatcccaagtggtcgaatgcgtacgattttaatttttctgagaccgaaaagtacaaggatattgcaggcttttatcgcgaagtggaggaacaaggatacaaggtttcattcgaatccgcctcaaaaaaggaggtcgacaaactcgtcgaagagggtaaactgtacatgttccaaatttacaataaagacttttcagacaaatcacacggaactcctaaccttcacacaatgtactttaaattgctgttcgatgaaaataatcacggtcaaattaggctgtcaggcggagctgagcttttcatgaggagggctagtctgaagaaagaggagctggtggtccatcctgcaaatagtcccatagctaataagaatcctgataaccctaagaaaaccaccactctctcctacgacgtttataaggataaacggttcagtgaagatcagtatgagttgcatattcccattgccataaataagtgccctaagaacatcttcaaaattaacacagaagtgagagttctcttgaaacacgatgataatccatatgtgattgggatagataggggagagcgtaacctcctttatattgtcgtggttgacggaaagggtaacatagtggagcaatacagcctcaatgaaattattaacaactttaatggtattagaataaagactgactatcatagtctcttggataaaaaagagaaggagaggttcgaagctaggcagaattggacgtctattgaaaatattaaagaactcaaagcagggtacattagccaagtcgttcacaagatatgcgagttggttgagaaatatgatgctgtcattgcactggaggatctcaatagcggtttcaaaaacagtcgtgttaaggtggagaagcaggtttaccagaaattcgagaagatgctgattgataagcttaactatatggtggacaaaaagtctaatccatgcgcgaccggtggcgcacttaagggctatcagatcacaaacaagttcgagtcgtttaagtccatgtcaacacagaacggtttcatcttctatatcccggcatggctgacctcaaaaattgatcctagcacggggttcgtaaacttacttaaaactaaatacacctcaattgctgattcaaaaaagtttatatcctcatttgaccgaattatgtatgtgcccgaggaggacctgttcgaattcgctctggactataagaacttttcaagaacagatgcggattatatcaagaagtggaaactttacagttatggtaaccgcattaggatattccggaaccccaaaaaaaataatgtctttgattgggaggaggtatgtctgacgtctgcttataaggagctatttaataagtacggcatcaattatcagcagggggacatccgcgcgcttctctgcgagcaatccgataaggctttctacagctccttcatggcattgatgagcctcatgctgcagatgagaaacagtatcacaggtagaacggacgtagacttcctaatttctccagtgaagaattcagatggcatcttctatgatagccgcaactatgaggcacaggagaacgccatcctgcccaaaaatgctgatgccaacggtgcgtataacattgctaggaaggtcctctgggccataggtcaattcaagaaagctgaagacgagaagctcgacaaggtaaaaattgccatatccaacaaggagtggctcgaatatgcacagacctctgtgaagcatggatctaagaagagaagaattaaacaagattga

SEQ ID NO: 7 - DNA, 2008 bp, Zea mays
  source 1..2008: mol_type = unassigned DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttctgcaggtc

SEQ ID NO: 8 - DNA, 298 bp, Oryza sativa
  source 1..298: mol_type = unassigned DNA
taatcgatcctccgatcccttaattaccataccattacaccatgcatcaatatccatatatatataaaccctttcgcacgtacttatactatgttttgtcatacatatatatgtgtcgaacgatcgatctatcactgatatgatatgattgatccatcagcctgatctctgtatcttgttatttgtataccgtcaaataaaagtttcttccacttgtgttaataattagctactctcatctcatgaaccctatatataactagtttaatttgctgtcaattgaacatgatgatcgatg

SEQ ID NO: 9 - DNA, 200 bp, Zea mays
  source 1..200: mol_type = unassigned DNA
cggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaatt

SEQ ID NO: 10 - DNA, 3681 bp, synthetic construct
  misc_feature 1..3681: note = Synthetic polynucleotide.Codon optimized LbCpf1-CO2
  source 1..3681: mol_type = other DNA
tcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcac

SEQ ID NO: 11 - DNA, 3681 bp, synthetic construct
  misc_feature 1..3681: note = Synthetic polynucleotide.Codon optimized LbCpf1-Os
  source 1..3681: mol_type = other DNA
tccaagctggagaagtttacaaactgttacagcctctccaaaaccctcaggtttaaagcgatcccggtgggcaagacccaggagaacatcgacaacaagaggctcctggtggaagacgagaagcgcgccgaagactacaagggcgtgaagaagctgctcgataggtactacctcagctttattaacgacgtgctgcacagcatcaaactcaagaatctcaacaactacatctccctcttccgcaaaaagacccgcaccgagaaggagaacaaggagctggagaacctggagatcaacctccgcaaggaaatcgccaaagcgttcaagggcaatgaagggtacaagagcctcttcaagaaagacatcatcgaaactatcctcccagagtttctcgatgacaaggacgagatcgcgctggtgaactcctttaacgggttcacaaccgcgtttaccggcttctttgataacagggaaaatatgttctccgaggaggccaagtccaccagcatcgccttcaggtgtatcaacgagaacctcacccgctacatttccaatatggacattttcgagaaggtggatgcgatcttcgataagcacgaggtgcaggagatcaaagagaagattctcaattccgattatgacgtcgaggatttcttcgaaggggagttctttaattttgtgctcacacaagagggcattgacgtgtacaacgcgattatcgggggcttcgtcacagagtccggggagaagattaaggggctgaatgagtacatcaatctgtacaatcagaagaccaagcagaaactgccgaaattcaagccgctctacaagcaagtcctgtccgatagggaaagcctctccttctacggcgagggctataccagcgacgaggaggtgctggaagtcttccgcaacacactgaataagaatagcgagattttctcctccatcaagaagctcgagaagctctttaagaactttgacgagtacagctccgccgggattttcgtgaagaacgggccggcgatcagcaccatctccaaggacatctttggcgagtggaacgtcatcagggacaagtggaacgccgagtacgacgacatccacctgaagaagaaggcggtggtgaccgagaagtatgaggacgatcgcaggaagtccttcaaaaaaatcggctccttcagcctcgaacagctccaggagtatgccgatgcggatctgtccgtcgtcgagaagctgaaggaaatcatcattcagaaggtcgacgagatctataaagtgtacgggtccagcgagaagctgttcgacgccgactttgtgctcgagaagtccctcaaaaagaatgacgccgtggtggccattatgaaagacctgctcgactccgtgaagtccttcgaaaattacattaaagcgttctttggggaggggaaggaaactaacagggatgagtccttctatggcgactttgtcctcgcgtacgacatcctgctgaaggtcgaccacatttacgacgcgatccgcaactacgtgacacagaagccgtactccaaagacaagttcaagctgtacttccagaacccgcaatttatggggggctgggacaaggataaagagacagactaccgcgcgacaattctccgctatggctccaaatactatctggccatcatggacaagaagtacgcgaagtgcctgcagaagatcgacaaagacgacgtcaatggcaactatgaaaagatcaactacaagctgctgccgggcccgaacaagatgctcccgaaggtgttcttcagcaagaagtggatggcctactacaatccaagcgaggatattcagaaaatctataaaaacgggaccttcaagaagggggacatgtttaacctcaacgactgccacaagctcattgatttcttcaaggatagcatttcccgctacccgaaatggtccaatgcgtacgattttaacttctccgagacagaaaagtacaaagacatcgcgggcttttacagggaggtggaggagcaagggtataaagtttcttttgaatccgcgagcaagaaggaagtcgacaagctcgtcgaggagggcaagctctacatgttccaaatttataacaaggacttttccgacaagagccatgggaccccaaacctccacaccatgtacttcaaactgctctttgacgagaacaaccacgggcaaatcaggctgagcggcggcgccgaattattcatgcgcagggcctccctcaagaaggaagagctggtcgtccatccagccaattccccgatcgcgaacaagaacccggacaatccgaaaaagaccaccaccctgtcctacgacgtctacaaggacaaacgcttcagcgaagaccagtacgaattacacatcccaattgcgattaataagtgcccaaagaatatcttcaaaattaatacagaggtcagggtgctgctcaaacacgacgacaatccgtatgtcatcggcattgacaggggcgagcgcaatctgctctatatcgtggtcgtggatgggaagggcaatattgtggagcagtactccctgaacgagattatcaacaacttcaatgggattaggattaagaccgactatcacagcctgctcgacaagaaagaaaaagagaggtttgaggcccgccaaaactggacctccattgagaatatcaaagaattaaaggccggctatatttcccaagtcgtccacaagatctgcgagctggtggagaaatatgacgccgtgattgcgctcgaagacttaaattctgggttcaagaactcccgcgtgaaggtggaaaaacaggtgtatcagaaattcgagaaaatgctgatcgacaaactcaattatatggtggataagaagtccaacccgtgtgccacagggggcgcgctgaagggctatcagatcaccaacaagttcgagagcttcaagagcatgagcacccagaacgggtttattttctacatcccggcgtggctcacctccaagattgacccgagcaccggcttcgtgaacctcctgaagacaaagtatacctccattgccgacagcaagaagtttatctcctccttcgaccgcattatgtatgtgccggaggaggacctcttcgagttcgccctcgactacaaaaacttcagccgcacagatgcggattacatcaagaagtggaagctgtactcctacgggaacaggatccgcatcttcaggaatccaaaaaaaaataacgtctttgactgggaggaagtgtgcctgacatccgcctacaaggaactgttcaataaatacggcatcaattaccagcagggcgacattcgcgccctcctctgtgagcagtccgacaaagcgttttactccagcttcatggccctcatgtccctgatgctccaaatgaggaatagcatcacagggcgcaccgacgtcgacttcctcatcagcccggtgaagaactccgacgggatcttttacgactcccgcaactatgaggcgcaagagaatgcgatcctcccgaagaacgccgatgcgaacggggcctataatatcgccaggaaagtgctctgggccatcgggcagttcaaaaaggcggaggatgagaagctcgacaaggtgaaaattgccatttccaacaaggagtggctggagtacgcgcagacctccgtgaagcac

SEQ ID NO: 12 - DNA, 3744 bp, synthetic construct
  misc_feature 1..3744: note = Synthetic polynucleotide.NLS-LbCpf1-CO2-NLS
  source 1..3744: mol_type = other DNA
ggatctaagaagagaagaattaaacaagattcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcacggatctaagaagagaagaattaaacaagattga

SEQ ID NO: 13 - DNA, 12 bp, synthetic construct
  misc_feature 1..12: note = Synthetic polynucleotide.kozak sequence
  source 1..12: mol_type = other DNA
gccgccatggcg

SEQ ID NO: 14 - DNA, 3756 bp, synthetic construct
  misc_feature 1..3756: note = Synthetic polynucleotide.kozak-NLS-LbCpf1-CO2-NLS
  source 1..3756: mol_type = other DNA
gccgccatggcgggatctaagaagagaagaattaaacaagattcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcacggatctaagaagagaagaattaaacaagattga

SEQ ID NO: 15 - DNA, 6062 bp, synthetic construct
  misc_feature 1..6062: note = Synthetic polynucleotide.Expression Cassette comprising the ZmUbiqitin promoter cassette, koz-NLS-LbCpf1-CO2-NLS cassette andthe Oryza sativa LTP termination sequence
  source 1..6062: mol_type = other DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttctgcaggtcgccgccatggcgggatctaagaagagaagaattaaacaagattcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcacggatctaagaagagaagaattaaacaagattgataatcgatcctccgatcccttaattaccataccattacaccatgcatcaatatccatatatatataaaccctttcgcacgtacttatactatgttttgtcatacatatatatgtgtcgaacgatcgatctatcactgatatgatatgattgatccatcagcctgatctctgtatcttgttatttgtataccgtcaaataaaagtttcttccacttgtgttaataattagctactctcatctcatgaaccctatatataactagtttaatttgctgtcaattgaacatgatgatcgatg

SEQ ID NO: 16 - DNA, 3744 bp, synthetic construct
  misc_feature 1..3744: note = Synthetic polynucleotide.NLS-LbCpf1-Os-NLS
  source 1..3744: mol_type = other DNA
ggatctaagaagagaagaattaaacaagattccaagctggagaagtttacaaactgttacagcctctccaaaaccctcaggtttaaagcgatcccggtgggcaagacccaggagaacatcgacaacaagaggctcctggtggaagacgagaagcgcgccgaagactacaagggcgtgaagaagctgctcgataggtactacctcagctttattaacgacgtgctgcacagcatcaaactcaagaatctcaacaactacatctccctcttccgcaaaaagacccgcaccgagaaggagaacaaggagctggagaacctggagatcaacctccgcaaggaaatcgccaaagcgttcaagggcaatgaagggtacaagagcctcttcaagaaagacatcatcgaaactatcctcccagagtttctcgatgacaaggacgagatcgcgctggtgaactcctttaacgggttcacaaccgcgtttaccggcttctttgataacagggaaaatatgttctccgaggaggccaagtccaccagcatcgccttcaggtgtatcaacgagaacctcacccgctacatttccaatatggacattttcgagaaggtggatgcgatcttcgataagcacgaggtgcaggagatcaaagagaagattctcaattccgattatgacgtcgaggatttcttcgaaggggagttctttaattttgtgctcacacaagagggcattgacgtgtacaacgcgattatcgggggcttcgtcacagagtccggggagaagattaaggggctgaatgagtacatcaatctgtacaatcagaagaccaagcagaaactgccgaaattcaagccgctctacaagcaagtcctgtccgatagggaaagcctctccttctacggcgagggctataccagcgacgaggaggtgctggaagtcttccgcaacacactgaataagaatagcgagattttctcctccatcaagaagctcgagaagctctttaagaactttgacgagtacagctccgccgggattttcgtgaagaacgggccggcgatcagcaccatctccaaggacatctttggcgagtggaacgtcatcagggacaagtggaacgccgagtacgacgacatccacctgaagaagaaggcggtggtgaccgagaagtatgaggacgatcgcaggaagtccttcaaaaaaatcggctccttcagcctcgaacagctccaggagtatgccgatgcggatctgtccgtcgtcgagaagctgaaggaaatcatcattcagaaggtcgacgagatctataaagtgtacgggtccagcgagaagctgttcgacgccgactttgtgctcgagaagtccctcaaaaagaatgacgccgtggtggccattatgaaagacctgctcgactccgtgaagtccttcgaaaattacattaaagcgttctttggggaggggaaggaaactaacagggatgagtccttctatggcgactttgtcctcgcgtacgacatcctgctgaaggtcgaccacatttacgacgcgatccgcaactacgtgacacagaagccgtactccaaagacaagttcaagctgtacttccagaacccgcaatttatggggggctgggacaaggataaagagacagactaccgcgcgacaattctccgctatggctccaaatactatctggccatcatggacaagaagtacgcgaagtgcctgcagaagatcgacaaagacgacgtcaatggcaactatgaaaagatcaactacaagctgctgccgggcccgaacaagatgctcccgaaggtgttcttcagcaagaagtggatggcctactacaatccaagcgaggatattcagaaaatctataaaaacgggaccttcaagaagggggacatgtttaacctcaacgactgccacaagctcattgatttcttcaaggatagcatttcccgctacccgaaatggtccaatgcgtacgattttaacttctccgagacagaaaagtacaaagacatcgcgggcttttacagggaggtggaggagcaagggtataaagtttcttttgaatccgcgagcaagaaggaagtcgacaagctcgtcgaggagggcaagctctacatgttccaaatttataacaaggacttttccgacaagagccatgggaccccaaacctccacaccatgtacttcaaactgctctttgacgagaacaaccacgggcaaatcaggctgagcggcggcgccgaattattcatgcgcagggcctccctcaagaaggaagagctggtcgtccatccagccaattccccgatcgcgaacaagaacccggacaatccgaaaaagaccaccaccctgtcctacgacgtctacaaggacaaacgcttcagcgaagaccagtacgaattacacatcccaattgcgattaataagtgcccaaagaatatcttcaaaattaatacagaggtcagggtgctgctcaaacacgacgacaatccgtatgtcatcggcattgacaggggcgagcgcaatctgctctatatcgtggtcgtggatgggaagggcaatattgtggagcagtactccctgaacgagattatcaacaacttcaatgggattaggattaagaccgactatcacagcctgctcgacaagaaagaaaaagagaggtttgaggcccgccaaaactggacctccattgagaatatcaaagaattaaaggccggctatatttcccaagtcgtccacaagatctgcgagctggtggagaaatatgacgccgtgattgcgctcgaagacttaaattctgggttcaagaactcccgcgtgaaggtggaaaaacaggtgtatcagaaattcgagaaaatgctgatcgacaaactcaattatatggtggataagaagtccaacccgtgtgccacagggggcgcgctgaagggctatcagatcaccaacaagttcgagagcttcaagagcatgagcacccagaacgggtttattttctacatcccggcgtggctcacctccaagattgacccgagcaccggcttcgtgaacctcctgaagacaaagtatacctccattgccgacagcaagaagtttatctcctccttcgaccgcattatgtatgtgccggaggaggacctcttcgagttcgccctcgactacaaaaacttcagccgcacagatgcggattacatcaagaagtggaagctgtactcctacgggaacaggatccgcatcttcaggaatccaaaaaaaaataacgtctttgactgggaggaagtgtgcctgacatccgcctacaaggaactgttcaataaatacggcatcaattaccagcagggcgacattcgcgccctcctctgtgagcagtccgacaaagcgttttactccagcttcatggccctcatgtccctgatgctccaaatgaggaatagcatcacagggcgcaccgacgtcgacttcctcatcagcccggtgaagaactccgacgggatcttttacgactcccgcaactatgaggcgcaagagaatgcgatcctcccgaagaacgccgatgcgaacggggcctataatatcgccaggaaagtgctctgggccatcgggcagttcaaaaaggcggaggatgagaagctcgacaaggtgaaaattgccatttccaacaaggagtggctggagtacgcgcagacctccgtgaagcacggatctaagaagagaagaattaaacaagattga

SEQ ID NO: 17 - DNA, 3756 bp, synthetic construct
  misc_feature 1..3756: note = Synthetic polynucleotide.kozak-NLS-LbCpf1-Os-NLS
  source 1..3756: mol_type = other DNA
gccgccatggcgggatctaagaagagaagaattaaacaagattccaagctggagaagtttacaaactgttacagcctctccaaaaccctcaggtttaaagcgatcccggtgggcaagacccaggagaacatcgacaacaagaggctcctggtggaagacgagaagcgcgccgaagactacaagggcgtgaagaagctgctcgataggtactacctcagctttattaacgacgtgctgcacagcatcaaactcaagaatctcaacaactacatctccctcttccgcaaaaagacccgcaccgagaaggagaacaaggagctggagaacctggagatcaacctccgcaaggaaatcgccaaagcgttcaagggcaatgaagggtacaagagcctcttcaagaaagacatcatcgaaactatcctcccagagtttctcgatgacaaggacgagatcgcgctggtgaactcctttaacgggttcacaaccgcgtttaccggcttctttgataacagggaaaatatgttctccgaggaggccaagtccaccagcatcgccttcaggtgtatcaacgagaacctcacccgctacatttccaatatggacattttcgagaaggtggatgcgatcttcgataagcacgaggtgcaggagatcaaagagaagattctcaattccgattatgacgtcgaggatttcttcgaaggggagttctttaattttgtgctcacacaagagggcattgacgtgtacaacgcgattatcgggggcttcgtcacagagtccggggagaagattaaggggctgaatgagtacatcaatctgtacaatcagaagaccaagcagaaactgccgaaattcaagccgctctacaagcaagtcctgtccgatagggaaagcctctccttctacggcgagggctataccagcgacgaggaggtgctggaagtcttccgcaacacactgaataagaatagcgagattttctcctccatcaagaagctcgagaagctctttaagaactttgacgagtacagctccgccgggattttcgtgaagaacgggccggcgatcagcaccatctccaaggacatctttggcgagtggaacgtcatcagggacaagtggaacgccgagtacgacgacatccacctgaagaagaaggcggtggtgaccgagaagtatgaggacgatcgcaggaagtccttcaaaaaaatcggctccttcagcctcgaacagctccaggagtatgccgatgcggatctgtccgtcgtcgagaagctgaaggaaatcatcattcagaaggtcgacgagatctataaagtgtacgggtccagcgagaagctgttcgacgccgactttgtgctcgagaagtccctcaaaaagaatgacgccgtggtggccattatgaaagacctgctcgactccgtgaagtccttcgaaaattacattaaagcgttctttggggaggggaaggaaactaacagggatgagtccttctatggcgactttgtcctcgcgtacgacatcctgctgaaggtcgaccacatttacgacgcgatccgcaactacgtgacacagaagccgtactccaaagacaagttcaagctgtacttccagaacccgcaatttatggggggctgggacaaggataaagagacagactaccgcgcgacaattctccgctatggctccaaatactatctggccatcatggacaagaagtacgcgaagtgcctgcagaagatcgacaaagacgacgtcaatggcaactatgaaaagatcaactacaagctgctgccgggcccgaacaagatgctcccgaaggtgttcttcagcaagaagtggatggcctactacaatccaagcgaggatattcagaaaatctataaaaacgggaccttcaagaagggggacatgtttaacctcaacgactgccacaagctcattgatttcttcaaggatagcatttcccgctacccgaaatggtccaatgcgtacgattttaacttctccgagacagaaaagtacaaagacatcgcgggcttttacagggaggtggaggagcaagggtataaagtttcttttgaatccgcgagcaagaaggaagtcgacaagctcgtcgaggagggcaagctctacatgttccaaatttataacaaggacttttccgacaagagccatgggaccccaaacctccacaccatgtacttcaaactgctctttgacgagaacaaccacgggcaaatcaggctgagcggcggcgccgaattattcatgcgcagggcctccctcaagaaggaagagctggtcgtccatccagccaattccccgatcgcgaacaagaacccggacaatccgaaaaagaccaccaccctgtcctacgacgtctacaaggacaaacgcttcagcgaagaccagtacgaattacacatcccaattgcgattaataagtgcccaaagaatatcttcaaaattaatacagaggtcagggtgctgctcaaacacgacgacaatccgtatgtcatcggcattgacaggggcgagcgcaatctgctctatatcgtggtcgtggatgggaagggcaatattgtggagcagtactccctgaacgagattatcaacaacttcaatgggattaggattaagaccgactatcacagcctgctcgacaagaaagaaaaagagaggtttgaggcccgccaaaactggacctccattgagaatatcaaagaattaaaggccggctatatttcccaagtcgtccacaagatctgcgagctggtggagaaatatgacgccgtgattgcgctcgaagacttaaattctgggttcaagaactcccgcgtgaaggtggaaaaacaggtgtatcagaaattcgagaaaatgctgatcgacaaactcaattatatggtggataagaagtccaacccgtgtgccacagggggcgcgctgaagggctatcagatcaccaacaagttcgagagcttcaagagcatgagcacccagaacgggtttattttctacatcccggcgtggctcacctccaagattgacccgagcaccggcttcgtgaacctcctgaagacaaagtatacctccattgccgacagcaagaagtttatctcctccttcgaccgcattatgtatgtgccggaggaggacctcttcgagttcgccctcgactacaaaaacttcagccgcacagatgcggattacatcaagaagtggaagctgtactcctacgggaacaggatccgcatcttcaggaatccaaaaaaaaataacgtctttgactgggaggaagtgtgcctgacatccgcctacaaggaactgttcaataaatacggcatcaattaccagcagggcgacattcgcgccctcctctgtgagcagtccgacaaagcgttttactccagcttcatggccctcatgtccctgatgctccaaatgaggaatagcatcacagggcgcaccgacgtcgacttcctcatcagcccggtgaagaactccgacgggatcttttacgactcccgcaactatgaggcgcaagagaatgcgatcctcccgaagaacgccgatgcgaacggggcctataatatcgccaggaaagtgctctgggccatcgggcagttcaaaaaggcggaggatgagaagctcgacaaggtgaaaattgccatttccaacaaggagtggctggagtacgcgcagacctccgtgaagcacggatctaagaagagaagaattaaacaagattga

SEQ ID NO: 18 - DNA, 6062 bp, synthetic construct
  misc_feature 1..6062: note = Synthetic polynucleotide.Expression Cassette comprising the ZmUbiqitin promoter cassette, koz-NLS-LbCpf1-Os-NLS cassette andthe Oryza sativa LTP termination sequence
  source 1..6062: mol_type = other DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttctgcaggtcgccgccatggcgggatctaagaagagaagaattaaacaagattccaagctggagaagtttacaaactgttacagcctctccaaaaccctcaggtttaaagcgatcccggtgggcaagacccaggagaacatcgacaacaagaggctcctggtggaagacgagaagcgcgccgaagactacaagggcgtgaagaagctgctcgataggtactacctcagctttattaacgacgtgctgcacagcatcaaactcaagaatctcaacaactacatctccctcttccgcaaaaagacccgcaccgagaaggagaacaaggagctggagaacctggagatcaacctccgcaaggaaatcgccaaagcgttcaagggcaatgaagggtacaagagcctcttcaagaaagacatcatcgaaactatcctcccagagtttctcgatgacaaggacgagatcgcgctggtgaactcctttaacgggttcacaaccgcgtttaccggcttctttgataacagggaaaatatgttctccgaggaggccaagtccaccagcatcgccttcaggtgtatcaacgagaacctcacccgctacatttccaatatggacattttcgagaaggtggatgcgatcttcgataagcacgaggtgcaggagatcaaagagaagattctcaattccgattatgacgtcgaggatttcttcgaaggggagttctttaattttgtgctcacacaagagggcattgacgtgtacaacgcgattatcgggggcttcgtcacagagtccggggagaagattaaggggctgaatgagtacatcaatctgtacaatcagaagaccaagcagaaactgccgaaattcaagccgctctacaagcaagtcctgtccgatagggaaagcctctccttctacggcgagggctataccagcgacgaggaggtgctggaagtcttccgcaacacactgaataagaatagcgagattttctcctccatcaagaagctcgagaagctctttaagaactttgacgagtacagctccgccgggattttcgtgaagaacgggccggcgatcagcaccatctccaaggacatctttggcgagtggaacgtcatcagggacaagtggaacgccgagtacgacgacatccacctgaagaagaaggcggtggtgaccgagaagtatgaggacgatcgcaggaagtccttcaaaaaaatcggctccttcagcctcgaacagctccaggagtatgccgatgcggatctgtccgtcgtcgagaagctgaaggaaatcatcattcagaaggtcgacgagatctataaagtgtacgggtccagcgagaagctgttcgacgccgactttgtgctcgagaagtccctcaaaaagaatgacgccgtggtggccattatgaaagacctgctcgactccgtgaagtccttcgaaaattacattaaagcgttctttggggaggggaaggaaactaacagggatgagtccttctatggcgactttgtcctcgcgtacgacatcctgctgaaggtcgaccacatttacgacgcgatccgcaactacgtgacacagaagccgtactccaaagacaagttcaagctgtacttccagaacccgcaatttatggggggctgggacaaggataaagagacagactaccgcgcgacaattctccgctatggctccaaatactatctggccatcatggacaagaagtacgcgaagtgcctgcagaagatcgacaaagacgacgtcaatggcaactatgaaaagatcaactacaagctgctgccgggcccgaacaagatgctcccgaaggtgttcttcagcaagaagtggatggcctactacaatccaagcgaggatattcagaaaatctataaaaacgggaccttcaagaagggggacatgtttaacctcaacgactgccacaagctcattgatttcttcaaggatagcatttcccgctacccgaaatggtccaatgcgtacgattttaacttctccgagacagaaaagtacaaagacatcgcgggcttttacagggaggtggaggagcaagggtataaagtttcttttgaatccgcgagcaagaaggaagtcgacaagctcgtcgaggagggcaagctctacatgttccaaatttataacaaggacttttccgacaagagccatgggaccccaaacctccacaccatgtacttcaaactgctctttgacgagaacaaccacgggcaaatcaggctgagcggcggcgccgaattattcatgcgcagggcctccctcaagaaggaagagctggtcgtccatccagccaattccccgatcgcgaacaagaacccggacaatccgaaaaagaccaccaccctgtcctacgacgtctacaaggacaaacgcttcagcgaagaccagtacgaattacacatcccaattgcgattaataagtgcccaaagaatatcttcaaaattaatacagaggtcagggtgctgctcaaacacgacgacaatccgtatgtcatcggcattgacaggggcgagcgcaatctgctctatatcgtggtcgtggatgggaagggcaatattgtggagcagtactccctgaacgagattatcaacaacttcaatgggattaggattaagaccgactatcacagcctgctcgacaagaaagaaaaagagaggtttgaggcccgccaaaactggacctccattgagaatatcaaagaattaaaggccggctatatttcccaagtcgtccacaagatctgcgagctggtggagaaatatgacgccgtgattgcgctcgaagacttaaattctgggttcaagaactcccgcgtgaaggtggaaaaacaggtgtatcagaaattcgagaaaatgctgatcgacaaactcaattatatggtggataagaagtccaacccgtgtgccacagggggcgcgctgaagggctatcagatcaccaacaagttcgagagcttcaagagcatgagcacccagaacgggtttattttctacatcccggcgtggctcacctccaagattgacccgagcaccggcttcgtgaacctcctgaagacaaagtatacctccattgccgacagcaagaagtttatctcctccttcgaccgcattatgtatgtgccggaggaggacctcttcgagttcgccctcgactacaaaaacttcagccgcacagatgcggattacatcaagaagtggaagctgtactcctacgggaacaggatccgcatcttcaggaatccaaaaaaaaataacgtctttgactgggaggaagtgtgcctgacatccgcctacaaggaactgttcaataaatacggcatcaattaccagcagggcgacattcgcgccctcctctgtgagcagtccgacaaagcgttttactccagcttcatggccctcatgtccctgatgctccaaatgaggaatagcatcacagggcgcaccgacgtcgacttcctcatcagcccggtgaagaactccgacgggatcttttacgactcccgcaactatgaggcgcaagagaatgcgatcctcccgaagaacgccgatgcgaacggggcctataatatcgccaggaaagtgctctgggccatcgggcagttcaaaaaggcggaggatgagaagctcgacaaggtgaaaattgccatttccaacaaggagtggctggagtacgcgcagacctccgtgaagcacggatctaagaagagaagaattaaacaagattgataatcgatcctccgatcccttaattaccataccattacaccatgcatcaatatccatatatatataaaccctttcgcacgtacttatactatgttttgtcatacatatatatgtgtcgaacgatcgatctatcactgatatgatatgattgatccatcagcctgatctctgtatcttgttatttgtataccgtcaaataaaagtttcttccacttgtgttaataattagctactctcatctcatgaaccctatatataactagtttaatttgctgtcaattgaacatgatgatcgatg

SEQ ID NO: 19 - DNA, 6072 bp, synthetic construct
  misc_feature 1..6072: note = Synthetic polynucleotide.Expression Cassette comprising the ZmUbiqitin promoter cassette, NLS-LbCpf1-Os-NLS cassette and theOryza sativa LTP termination sequence
  source 1..6072: mol_type = other DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttctgcaggtccggtaccatgggatctaagaagagaagaattaaacaagatatgtccaagctggagaagtttacaaactgttacagcctctccaaaaccctcaggtttaaagcgatcccggtgggcaagacccaggagaacatcgacaacaagaggctcctggtggaagacgagaagcgcgccgaagactacaagggcgtgaagaagctgctcgataggtactacctcagctttattaacgacgtgctgcacagcatcaaactcaagaatctcaacaactacatctccctcttccgcaaaaagacccgcaccgagaaggagaacaaggagctggagaacctggagatcaacctccgcaaggaaatcgccaaagcgttcaagggcaatgaagggtacaagagcctcttcaagaaagacatcatcgaaactatcctcccagagtttctcgatgacaaggacgagatcgcgctggtgaactcctttaacgggttcacaaccgcgtttaccggcttctttgataacagggaaaatatgttctccgaggaggccaagtccaccagcatcgccttcaggtgtatcaacgagaacctcacccgctacatttccaatatggacattttcgagaaggtggatgcgatcttcgataagcacgaggtgcaggagatcaaagagaagattctcaattccgattatgacgtcgaggatttcttcgaaggggagttctttaattttgtgctcacacaagagggcattgacgtgtacaacgcgattatcgggggcttcgtcacagagtccggggagaagattaaggggctgaatgagtacatcaatctgtacaatcagaagaccaagcagaaactgccgaaattcaagccgctctacaagcaagtcctgtccgatagggaaagcctctccttctacggcgagggctataccagcgacgaggaggtgctggaagtcttccgcaacacactgaataagaatagcgagattttctcctccatcaagaagctcgagaagctctttaagaactttgacgagtacagctccgccgggattttcgtgaagaacgggccggcgatcagcaccatctccaaggacatctttggcgagtggaacgtcatcagggacaagtggaacgccgagtacgacgacatccacctgaagaagaaggcggtggtgaccgagaagtatgaggacgatcgcaggaagtccttcaaaaaaatcggctccttcagcctcgaacagctccaggagtatgccgatgcggatctgtccgtcgtcgagaagctgaaggaaatcatcattcagaaggtcgacgagatctataaagtgtacgggtccagcgagaagctgttcgacgccgactttgtgctcgagaagtccctcaaaaagaatgacgccgtggtggccattatgaaagacctgctcgactccgtgaagtccttcgaaaattacattaaagcgttctttggggaggggaaggaaactaacagggatgagtccttctatggcgactttgtcctcgcgtacgacatcctgctgaaggtcgaccacatttacgacgcgatccgcaactacgtgacacagaagccgtactccaaagacaagttcaagctgtacttccagaacccgcaatttatggggggctgggacaaggataaagagacagactaccgcgcgacaattctccgctatggctccaaatactatctggccatcatggacaagaagtacgcgaagtgcctgcagaagatcgacaaagacgacgtcaatggcaactatgaaaagatcaactacaagctgctgccgggcccgaacaagatgctcccgaaggtgttcttcagcaagaagtggatggcctactacaatccaagcgaggatattcagaaaatctataaaaacgggaccttcaagaagggggacatgtttaacctcaacgactgccacaagctcattgatttcttcaaggatagcatttcccgctacccgaaatggtccaatgcgtacgattttaacttctccgagacagaaaagtacaaagacatcgcgggcttttacagggaggtggaggagcaagggtataaagtttcttttgaatccgcgagcaagaaggaagtcgacaagctcgtcgaggagggcaagctctacatgttccaaatttataacaaggacttttccgacaagagccatgggaccccaaacctccacaccatgtacttcaaactgctctttgacgagaacaaccacgggcaaatcaggctgagcggcggcgccgaattattcatgcgcagggcctccctcaagaaggaagagctggtcgtccatccagccaattccccgatcgcgaacaagaacccggacaatccgaaaaagaccaccaccctgtcctacgacgtctacaaggacaaacgcttcagcgaagaccagtacgaattacacatcccaattgcgattaataagtgcccaaagaatatcttcaaaattaatacagaggtcagggtgctgctcaaacacgacgacaatccgtatgtcatcggcattgacaggggcgagcgcaatctgctctatatcgtggtcgtggatgggaagggcaatattgtggagcagtactccctgaacgagattatcaacaacttcaatgggattaggattaagaccgactatcacagcctgctcgacaagaaagaaaaagagaggtttgaggcccgccaaaactggacctccattgagaatatcaaagaattaaaggccggctatatttcccaagtcgtccacaagatctgcgagctggtggagaaatatgacgccgtgattgcgctcgaagacttaaattctgggttcaagaactcccgcgtgaaggtggaaaaacaggtgtatcagaaattcgagaaaatgctgatcgacaaactcaattatatggtggataagaagtccaacccgtgtgccacagggggcgcgctgaagggctatcagatcaccaacaagttcgagagcttcaagagcatgagcacccagaacgggtttattttctacatcccggcgtggctcacctccaagattgacccgagcaccggcttcgtgaacctcctgaagacaaagtatacctccattgccgacagcaagaagtttatctcctccttcgaccgcattatgtatgtgccggaggaggacctcttcgagttcgccctcgactacaaaaacttcagccgcacagatgcggattacatcaagaagtggaagctgtactcctacgggaacaggatccgcatcttcaggaatccaaaaaaaaataacgtctttgactgggaggaagtgtgcctgacatccgcctacaaggaactgttcaataaatacggcatcaattaccagcagggcgacattcgcgccctcctctgtgagcagtccgacaaagcgttttactccagcttcatggccctcatgtccctgatgctccaaatgaggaatagcatcacagggcgcaccgacgtcgacttcctcatcagcccggtgaagaactccgacgggatcttttacgactcccgcaactatgaggcgcaagagaatgcgatcctcccgaagaacgccgatgcgaacggggcctataatatcgccaggaaagtgctctgggccatcgggcagttcaaaaaggcggaggatgagaagctcgacaaggtgaaaattgccatttccaacaaggagtggctggagtacgcgcagacctccgtgaagcacggatctaagaagagaagaattaaacaagattgattaattaatcgatcctccgatcccttaattaccatgccattacaccatgcatcaatatccatatatatataaaccctttcgcacgtacttatactatgttttgtcatacatatatatgtgtcgaacgatcgatctatcactgatatgatatgattgatccatcagcctgatctctgtatcttgttatttgtataccgtcaaataaaagtttcttccacttgtgttaataattagctactctcatctcatgaaccctatatataactagtttaatttgctgtcaattgaacatgatgatcgatgcctg

SEQ ID NO: 20 - DNA, 5091 bp, synthetic construct
  misc_feature 1..5091: note = Synthetic polynucleotide.Expression Cassette comprising the DaMVpromoter cassette, NLS-LbCpf1-CO2-NLS cassette and a Medicagotruncatula termination sequence
  source 1..5091: mol_type = other DNA
agacatcctggaccaatatgctgaagattatgctacctacaccaggataggacttgaagcacttaaccttgaagattggttcgaagaaccagaacccgatccacctaaccctgtggaccgccagaggatagaggacatcctggacctactgaacgtcagcaatgacgactgaaagattcccaggacaccggcggaagtggtggacccagtctaggtgcgatgcttagtcgcgcacgatgactatgtcggaaggcatctttgctttcggcaaactttagtaatactttaaggaaagtattgtacaagttaggtgcagagacaataatgcacccagctttagctttgtttatggaattattgtgtcggttgcattattggatgcctgcgtgcaccctaagcaatcaacggagaaacaaagataaaaatcaattactcacatgaaagagtattgatcacgagtcactatggagcgacaatctccagacaggatgtcagcatcttatcttcctttgaagaaagcatcatcaataacgatgtaatggtggggacatccactaagttattgctctgcaaacagctcaaaaagctactggccgacaatcataattgctcggcatgtgcaggtggggcctccactagcaataatacaagctttacagcttgcagtgactcatcctccaataatggagaaaaagacgtcagcagtgacgaacaagggtcgaaagacttgcctatataagggcattctcccctcagttgaagatcatcgaaagttggagcaataaactctctcttcaacaaatctatcttttatcttttatcggtaccaaaaaatggcgggatctaagaagagaagaattaaacaagattcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcacggatctaagaagagaagaattaaacaagattgacttaattaaagggctctctgtcatgatttcatactttcattattgagctctgtaattacaattatgaccatgagaacatctcttattgtgtggccttttaattgctgatgttagtactgaaccaaagcttatcgtgatgatgtaaaagcaataagtacttgtttgtagcttctttgtgtctccctttgggcttaatacatctgtttagtgttgtggctttggcatagacttctcttggtaataatgccttgcaatgcaaaatttcaattatcaaattctattatgttctcaccttatggtaacagcttaccctgtggaagatgagattcttgagttgagtcattgccaatttttggcattagcttttgaattagtgaattttgacaaaaattaccgtgacactgattttgttgaagctcttaagtgtagtttttacaaaatttcagtggctcgttgtgattatgtcaaactcacggcgaatgtagttcttacagaatttcagtggctc

SEQ ID NO: 21 - DNA, 5091 bp, synthetic construct
  misc_feature 1..5091: note = Synthetic polynucleotide.Expression Cassette comprising the DaMVpromoter cassette, NLS-LbCpf1-Os-NLS cassette and a Medicagotruncatula termination sequence
  source 1..5091: mol_type = other DNA
agacatcctggaccaatatgctgaagattatgctacctacaccaggataggacttgaagcacttaaccttgaagattggttcgaagaaccagaacccgatccacctaaccctgtggaccgccagaggatagaggacatcctggacctactgaacgtcagcaatgacgactgaaagattcccaggacaccggcggaagtggtggacccagtctaggtgcgatgcttagtcgcgcacgatgactatgtcggaaggcatctttgctttcggcaaactttagtaatactttaaggaaagtattgtacaagttaggtgcagagacaataatgcacccagctttagctttgtttatggaattattgtgtcggttgcattattggatgcctgcgtgcaccctaagcaatcaacggagaaacaaagataaaaatcaattactcacatgaaagagtattgatcacgagtcactatggagcgacaatctccagacaggatgtcagcatcttatcttcctttgaagaaagcatcatcaataacgatgtaatggtggggacatccactaagttattgctctgcaaacagctcaaaaagctactggccgacaatcataattgctcggcatgtgcaggtggggcctccactagcaataatacaagctttacagcttgcagtgactcatcctccaataatggagaaaaagacgtcagcagtgacgaacaagggtcgaaagacttgcctatataagggcattctcccctcagttgaagatcatcgaaagttggagcaataaactctctcttcaacaaatctatcttttatcttttatcggtaccaaaaaatggcgggatctaagaagagaagaattaaacaagattccaagctggagaagtttacaaactgttacagcctctccaaaaccctcaggtttaaagcgatcccggtgggcaagacccaggagaacatcgacaacaagaggctcctggtggaagacgagaagcgcgccgaagactacaagggcgtgaagaagctgctcgataggtactacctcagctttattaacgacgtgctgcacagcatcaaactcaagaatctcaacaactacatctccctcttccgcaaaaagacccgcaccgagaaggagaacaaggagctggagaacctggagatcaacctccgcaaggaaatcgccaaagcgttcaagggcaatgaagggtacaagagcctcttcaagaaagacatcatcgaaactatcctcccagagtttctcgatgacaaggacgagatcgcgctggtgaactcctttaacgggttcacaaccgcgtttaccggcttctttgataacagggaaaatatgttctccgaggaggccaagtccaccagcatcgccttcaggtgtatcaacgagaacctcacccgctacatttccaatatggacattttcgagaaggtggatgcgatcttcgataagcacgaggtgcaggagatcaaagagaagattctcaattccgattatgacgtcgaggatttcttcgaaggggagttctttaattttgtgctcacacaagagggcattgacgtgtacaacgcgattatcgggggcttcgtcacagagtccggggagaagattaaggggctgaatgagtacatcaatctgtacaatcagaagaccaagcagaaactgccgaaattcaagccgctctacaagcaagtcctgtccgatagggaaagcctctccttctacggcgagggctataccagcgacgaggaggtgctggaagtcttccgcaacacactgaataagaatagcgagattttctcctccatcaagaagctcgagaagctctttaagaactttgacgagtacagctccgccgggattttcgtgaagaacgggccggcgatcagcaccatctccaaggacatctttggcgagtggaacgtcatcagggacaagtggaacgccgagtacgacgacatccacctgaagaagaaggcggtggtgaccgagaagtatgaggacgatcgcaggaagtccttcaaaaaaatcggctccttcagcctcgaacagctccaggagtatgccgatgcggatctgtccgtcgtcgagaagctgaaggaaatcatcattcagaaggtcgacgagatctataaagtgtacgggtccagcgagaagctgttcgacgccgactttgtgctcgagaagtccctcaaaaagaatgacgccgtggtggccattatgaaagacctgctcgactccgtgaagtccttcgaaaattacattaaagcgttctttggggaggggaaggaaactaacagggatgagtccttctatggcgactttgtcctcgcgtacgacatcctgctgaaggtcgaccacatttacgacgcgatccgcaactacgtgacacagaagccgtactccaaagacaagttcaagctgtacttccagaacccgcaatttatggggggctgggacaaggataaagagacagactaccgcgcgacaattctccgctatggctccaaatactatctggccatcatggacaagaagtacgcgaagtgcctgcagaagatcgacaaagacgacgtcaatggcaactatgaaaagatcaactacaagctgctgccgggcccgaacaagatgctcccgaaggtgttcttcagcaagaagtggatggcctactacaatccaagcgaggatattcagaaaatctataaaaacgggaccttcaagaagggggacatgtttaacctcaacgactgccacaagctcattgatttcttcaaggatagcatttcccgctacccgaaatggtccaatgcgtacgattttaacttctccgagacagaaaagtacaaagacatcgcgggcttttacagggaggtggaggagcaagggtataaagtttcttttgaatccgcgagcaagaaggaagtcgacaagctcgtcgaggagggcaagctctacatgttccaaatttataacaaggacttttccgacaagagccatgggaccccaaacctccacaccatgtacttcaaactgctctttgacgagaacaaccacgggcaaatcaggctgagcggcggcgccgaattattcatgcgcagggcctccctcaagaaggaagagctggtcgtccatccagccaattccccgatcgcgaacaagaacccggacaatccgaaaaagaccaccaccctgtcctacgacgtctacaaggacaaacgcttcagcgaagaccagtacgaattacacatcccaattgcgattaataagtgcccaaagaatatcttcaaaattaatacagaggtcagggtgctgctcaaacacgacgacaatccgtatgtcatcggcattgacaggggcgagcgcaatctgctctatatcgtggtcgtggatgggaagggcaatattgtggagcagtactccctgaacgagattatcaacaacttcaatgggattaggattaagaccgactatcacagcctgctcgacaagaaagaaaaagagaggtttgaggcccgccaaaactggacctccattgagaatatcaaagaattaaaggccggctatatttcccaagtcgtccacaagatctgcgagctggtggagaaatatgacgccgtgattgcgctcgaagacttaaattctgggttcaagaactcccgcgtgaaggtggaaaaacaggtgtatcagaaattcgagaaaatgctgatcgacaaactcaattatatggtggataagaagtccaacccgtgtgccacagggggcgcgctgaagggctatcagatcaccaacaagttcgagagcttcaagagcatgagcacccagaacgggtttattttctacatcccggcgtggctcacctccaagattgacccgagcaccggcttcgtgaacctcctgaagacaaagtatacctccattgccgacagcaagaagtttatctcctccttcgaccgcattatgtatgtgccggaggaggacctcttcgagttcgccctcgactacaaaaacttcagccgcacagatgcggattacatcaagaagtggaagctgtactcctacgggaacaggatccgcatcttcaggaatccaaaaaaaaataacgtctttgactgggaggaagtgtgcctgacatccgcctacaaggaactgttcaataaatacggcatcaattaccagcagggcgacattcgcgccctcctctgtgagcagtccgacaaagcgttttactccagcttcatggccctcatgtccctgatgctccaaatgaggaatagcatcacagggcgcaccgacgtcgacttcctcatcagcccggtgaagaactccgacgggatcttttacgactcccgcaactatgaggcgcaagagaatgcgatcctcccgaagaacgccgatgcgaacggggcctataatatcgccaggaaagtgctctgggccatcgggcagttcaaaaaggcggaggatgagaagctcgacaaggtgaaaattgccatttccaacaaggagtggctggagtacgcgcagacctccgtgaagcacggatctaagaagagaagaattaaacaagattgacttaattaaagggctctctgtcatgatttcatactttcattattgagctctgtaattacaattatgaccatgagaacatctcttattgtgtggccttttaattgctgatgttagtactgaaccaaagcttatcgtgatgatgtaaaagcaataagtacttgtttgtagcttctttgtgtctccctttgggcttaatacatctgtttagtgttgtggctttggcatagacttctcttggtaataatgccttgcaatgcaaaatttcaattatcaaattctattatgttctcaccttatggtaacagcttaccctgtggaagatgagattcttgagttgagtcattgccaatttttggcattagcttttgaattagtgaattttgacaaaaattaccgtgacactgattttgttgaagctcttaagtgtagtttttacaaaatttcagtggctcgttgtgattatgtcaaactcacggcgaatgtagttcttacagaatttcagtggctc

SEQ ID NO: 22 - DNA, 1000 bp, Medicago truncatula
  source 1..1000: mol_type = unassigned DNA
actgttaataatttttaaacgtcagcgcactaaaaaaacgaaaagacggacacgtgaaaataaaaaacacacactagtttatgacgcaatactattttacttatgatttgggtacattagacaaaaccgtgaaagagatgtatcagctatgaaacctgtatacttcaatacagagacgtactcatatcggatacgtacgcacgaagtatcatattaattattttaatttttaataaatattttatcggatacttatgtgatactctacatatacacaaggatatttctaagatactttatagatacgtatcctagaaaaacatgaagagtaaaaaagtgagacaatgttgtaaaaattcattataaatgtatatgattcaattttagatatgcatcagtataattgattctcgatgaaacacttaaaattatatttcttgtggaagaacgtagcgagagaggtgattcagttagacaacattaaataaaattaatgttaagttcttttaatgatgtttctctcaatatcacatcatatgaaaatgtaatatgatttataagaaaatttttaaaaaatttattttaataatcacatgtactattttttaaaaattgtatcttttataataatacaataataaagagtaatcagtgttaatttttcttcaaatataagttttattataaatcattgttaacgtatcataagtcattaccgtatcgtatcttaatttttttttaaaaaccgctaattcacgtacccgtattgtattgtacccgcacctgtatcacaatcgatcttagttagaagaattgtctcgaggcggtgcaagacagcatataatagacgtggactctcttataccaaacgttgtcgtatcacaaagggttaggtaacaagtcacagtttgtccacgtgtcacgttttaattggaagagctgccgttggcgtaatataacagccaatcgatttttgctataaaagcaaatcaggtaaact

SEQ ID NO: 23 - DNA, 92 bp, Medicago truncatula
  source 1..92: mol_type = unassigned DNA
aaacttcttcattcttttcttccccatcgctacaaaaccggttcctttggaaaagagattcattcaaacctagcacccaattccgtttcaag

SEQ ID NO: 24 - DNA, 499 bp, Medicago truncatula
  source 1..499: mol_type = unassigned DNA
gtataatctactttctattcttcgattattttattattattagctactatcgtttaatcgatcttttcttttgatccgtcaaatttaaattcaattagggttttgttcttttctttcatctgattgaaatccttctgaattgaaccgtttacttgattttactgtttattgtatgatttaatcctttgtttttcaaagacagtctttagattgtgattaggggttcatataaatttttagatttggatttttgtattgtatgattcaaaaaatacgtcctttaattagattagtacatggatattttttacccgatttattgattgtcagggagaatttgatgagcaagtttttttgatgtctgttgtaaattgaattgattataattgctgatctgctgcttccagttttcataacccatattcttttaaccttgttgtacacacaatgaaaaattggtgattgattcatttgtttttctttgttttggattatacag

SEQ ID NO: 25 - DNA, 500 bp, Medicago truncatula
  source 1..500: mol_type = unassigned DNA
ttaatcatctgaaactgttcaccatgcatgcaatcttgtgaaatatatggttttaattagacttcaatcttatgttggctattgtactaataaaagcatgtcatgttattttcatttgattttatctgtactttggtttgtttgaagaataaagatgagcttgctatgcatgcatgcatgccatcgattatcagggtttccttttttcttttctggcttcccatcaatttggtgtgaattagtgtgtgtgatatattatattatgctatttatgaaataaattgttggttatatttgatctacaatctacatacatgtgatttttatcaacaaaatatctcgggaaacaatacctttttggtagcaaaattcaaataatactattttaaataaatcaaagttaaccaataccttattcaagttggaggggtctcaaacaagcaaaagaattcaagttgttaatgaacttcggttaatgataaaagaattcgcatttaaaa

SEQ ID NO: 26 - DNA, 5856 bp, synthetic construct
  misc_feature 1..5856: note = Synthetic polynucleotide.Expression cassette comprising Medicagotruncatula promoter cassette, NLS-LbCpf1-CO2-NLS and Medicagotruncatula termination sequence
  source 1..5856: mol_type = other DNA
actgttaataatttttaaacgtcagcgcactaaaaaaacgaaaagacggacacgtgaaaataaaaaacacacactagtttatgacgcaatactattttacttatgatttgggtacattagacaaaaccgtgaaagagatgtatcagctatgaaacctgtatacttcaatacagagacgtactcatatcggatacgtacgcacgaagtatcatattaattattttaatttttaataaatattttatcggatacttatgtgatactctacatatacacaaggatatttctaagatactttatagatacgtatcctagaaaaacatgaagagtaaaaaagtgagacaatgttgtaaaaattcattataaatgtatatgattcaattttagatatgcatcagtataattgattctcgatgaaacacttaaaattatatttcttgtggaagaacgtagcgagagaggtgattcagttagacaacattaaataaaattaatgttaagttcttttaatgatgtttctctcaatatcacatcatatgaaaatgtaatatgatttataagaaaatttttaaaaaatttattttaataatcacatgtactattttttaaaaattgtatcttttataataatacaataataaagagtaatcagtgttaatttttcttcaaatataagttttattataaatcattgttaacgtatcataagtcattaccgtatcgtatcttaatttttttttaaaaaccgctaattcacgtacccgtattgtattgtacccgcacctgtatcacaatcgatcttagttagaagaattgtctcgaggcggtgcaagacagcatataatagacgtggactctcttataccaaacgttgtcgtatcacaaagggttaggtaacaagtcacagtttgtccacgtgtcacgttttaattggaagagctgccgttggcgtaatataacagccaatcgatttttgctataaaagcaaatcaggtaaactaaacttcttcattcttttcttccccatcgctacaaaaccggttcctttggaaaagagattcattcaaacctagcacccaattccgtttcaaggtataatctactttctattcttcgattattttattattattagctactatcgtttaatcgatcttttcttttgatccgtcaaatttaaattcaattagggttttgttcttttctttcatctgattgaaatccttctgaattgaaccgtttacttgattttactgtttattgtatgatttaatcctttgtttttcaaagacagtctttagattgtgattaggggttcatataaatttttagatttggatttttgtattgtatgattcaaaaaatacgtcctttaattagattagtacatggatattttttacccgatttattgattgtcagggagaatttgatgagcaagtttttttgatgtctgttgtaaattgaattgattataattgctgatctgctgcttccagttttcataacccatattcttttaaccttgttgtacacacaatgaaaaattggtgattgattcatttgtttttctttgttttggattatacagggtaccaaaaaatggcgggatctaagaagagaagaattaaacaagattcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcacggatctaagaagagaagaattaaacaagattgattaattaatcatctgaaactgttcaccatgcatgcaatcttgtgaaatatatggttttaattagacttcaatcttatgttggctattgtactaataaaagcatgtcatgttattttcatttgattttatctgtactttggtttgtttgaagaataaagatgagcttgctatgcatgcatgcatgccatcgattatcagggtttccttttttcttttctggcttcccatcaatttggtgtgaattagtgtgtgtgatatattatattatgctatttatgaaataaattgttggttatatttgatctacaatctacatacatgtgatttttatcaacaaaatatctcgggaaacaatacctttttggtagcaaaattcaaataatactattttaaataaatcaaagttaaccaataccttattcaagttggaggggtctcaaacaagcaaaagaattcaagttgttaatgaacttcggttaatgataaaagaattcgcatttaaaa

SEQ ID NO: 27 - DNA, 617 bp, Cucumis melo
  source 1..617: mol_type = unassigned DNA
aaattttaataattaaaatgaacaatttttcaagagtaatagagtttgagagatgtcagagaagtttgaggaagaagataacaagtgggagaagagaataagtttgttgtgtgaaagagaaggggaaatttcattcaagggtatattgaactttttactcaaattttgtaagtctattttttccgatcaatcctaaaatcacacacacccttaaaaaatggattatatttggcaattttccatgataaactcatttttaatttagagttattttttcaacgagatattaacagttttagttcatatactaattgtaagaatagtttcttttaagttgaatagaatttttgaaacttttaatagttcaaaaggtatttttgaaacaaaataagaatgtttttgaactttttataaaaagaattgagatttttttgaaatttttgataaagagaaaagaaaagaagaaagaaaaaagaaaaacaagtttgtagaactccgtgggaaaatcgtcgagggccctgtgaaggaattttgaaattataatgagggtattttcgtcaacaagggaatttagacatcgtatataagcatcctcaaaccctataattaagcccttc

SEQ ID NO: 28 - DNA, 54 bp, Cucumis melo
  source 1..54: mol_type = unassigned DNA
aatccaattgccattctccatctctcgccgcaagggtttaagagcagcttctct

SEQ ID NO: 29 - DNA, 545 bp, Cucumis melo
  source 1..545: mol_type = unassigned DNA
cctcaggttggggtttccccctatcttcttcattcttcctcttctcgatttctttcttctatttgctcgatagtctcttatttcttgagcttttgctgtttttctcctgtacatcctaacatgaattataacttggttttgattttgtcttttacttctgtattaaacaacttttcttacccttttattcttctcttcttcttcgtgtccctgcccttttgtttttatgctaattttatgtttctgtttatcaatctatcgaggcgtgacctgtcgttcttccaatagcgtagatctgcacttaatctattctagctgattggattggtcgtttttcgtttttttaatttattttctctgttctagttccgataaatttttttatatataattaacaagttctccagccaaaagggttaatattgcgttggatattttaatttttacgttatttagatgtgtgaatctaataaaattagggttattcataaatttcagtaatgatattttggttatctgttcttgctgttcctgtttcgcagttc

SEQ ID NO: 30 - DNA, 19 bp, Cucumis melo
  source 1..19: mol_type = unassigned DNA
ttttacctaatattcaagc

SEQ ID NO: 31 - DNA, 5500 bp, synthetic construct
  misc_feature 1..5500: note = Synthetic polynucleotide.Expression cassette comprising Cucumismelo EF1a promoter cassette, NLS-LbCpf1-CO2-NLS and Medicagotruncatula termination sequence
  source 1..5500: mol_type = other DNA
aaattttaataattaaaatgaacaatttttcaagagtaatagagtttgagagatgtcagagaagtttgaggaagaagataacaagtgggagaagagaataagtttgttgtgtgaaagagaaggggaaatttcattcaagggtatattgaactttttactcaaattttgtaagtctattttttccgatcaatcctaaaatcacacacacccttaaaaaatggattatatttggcaattttccatgataaactcatttttaatttagagttattttttcaacgagatattaacagttttagttcatatactaattgtaagaatagtttcttttaagttgaatagaatttttgaaacttttaatagttcaaaaggtatttttgaaacaaaataagaatgtttttgaactttttataaaaagaattgagatttttttgaaatttttgataaagagaaaagaaaagaagaaagaaaaaagaaaaacaagtttgtagaactccgtgggaaaatcgtcgagggccctgtgaaggaattttgaaattataatgagggtattttcgtcaacaagggaatttagacatcgtatataagcatcctcaaaccctataattaagcccttcaatccaattgccattctccatctctcgccgcaagggtttaagagcagcttctctcctcaggttggggtttccccctatcttcttcattcttcctcttctcgatttctttcttctatttgctcgatagtctcttatttcttgagcttttgctgtttttctcctgtacatcctaacatgaattataacttggttttgattttgtcttttacttctgtattaaacaacttttcttacccttttattcttctcttcttcttcgtgtccctgcccttttgtttttatgctaattttatgtttctgtttatcaatctatcgaggcgtgacctgtcgttcttccaatagcgtagatctgcacttaatctattctagctgattggattggtcgtttttcgtttttttaatttattttctctgttctagttccgataaatttttttatatataattaacaagttctccagccaaaagggttaatattgcgttggatattttaatttttacgttatttagatgtgtgaatctaataaaattagggttattcataaatttcagtaatgatattttggttatctgttcttgctgttcctgtttcgcagttcttttacctaatattcaagcggtaccaaaaaatggcgggatctaagaagagaagaattaaacaagattcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcacggatctaagaagagaagaattaaacaagattgattaattaatcatctgaaactgttcaccatgcatgcaatcttgtgaaatatatggttttaattagacttcaatcttatgttggctattgtactaataaaagcatgtcatgttattttcatttgattttatctgtactttggtttgtttgaagaataaagatgagcttgctatgcatgcatgcatgccatcgattatcagggtttccttttttcttttctggcttcccatcaatttggtgtgaattagtgtgtgtgatatattatattatgctatttatgaaataaattgttggttatatttgatctacaatctacatacatgtgatttttatcaacaaaatatctcgggaaacaatacctttttggtagcaaaattcaaataatactattttaaataaatcaaagttaaccaataccttattcaagttggaggggtctcaaacaagcaaaagaattcaagttgttaatgaacttcggttaatgataaaagaattcgcatttaaaa

SEQ ID NO: 32 - DNA, 829 bp, Arabidopsis thaliana
  source 1..829: mol_type = unassigned DNA
gttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttctt

SEQ ID NO: 33 - DNA, 67 bp, Arabidopsis thaliana
  source 1..67: mol_type = unassigned DNA
cacaattcagatttcaatttctcaaaatcttaaaaactttctctcaattctctctaccgtgatcaag

SEQ ID NO: 34 - DNA, 306 bp, Arabidopsis thaliana
  source 1..306: mol_type = unassigned DNA
gtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggt

SEQ ID NO: 35 - DNA, 315 bp, Gossypium barbadense
  source 1..315: mol_type = unassigned DNA
accatatgacactggtgcatgtgccatcatcatgcagtaatttcatggtatatcttaattatatggttaataaaaaaaagatggtgagtgaataatgtgcgtgcattcctccatgcaccaatggtgaatctctttgcatacatagagattctgaatgattatagtttatgttgtagtgaaattaattttgaatgttgtttttaaattttaatgtcacttggcttgatttatgttttaacgaagcttatgttatgtattttactttaatgatattgcatgtattgttaatttaacattgcttgatcagtatactct

SEQ ID NO: 36 - DNA, 5292 bp, synthetic construct
  misc_feature 1..5292: note = Synthetic polynucleotide.Expression cassette comprisingArabidopsis thaliana Ubiquitin promoter cassette,NLS-LbCpf1-CO2-NLS and Gossypium barbadense termination sequence.
  source 1..5292: mol_type = other DNA
gttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggtggtaccaaaaaatggcgggatctaagaagagaagaattaaacaagattcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcacggatctaagaagagaagaattaaacaagattgattaattaagggcccaccatatgacactggtgcatgtgccatcatcatgcagtaatttcatggtatatcttaattatatggttaataaaaaaaagatggtgagtgaataatgtgcgtgcattcctccatgcaccaatggtgaatctctttgcatacatagagattctgaatgattatagtttatgttgtagtgaaattaattttgaatgttgtttttaaattttaatgtcacttggcttgatttatgttttaacgaagcttatgttatgtattttactttaatgatattgcatgtattgttaatttaacattgcttgatcagtatactct

SEQ ID NO: 37 - DNA, 822 bp, synthetic construct
  misc_feature 1..822: note = Synthetic polynucleotide.Chimeric promoter cassette comprisingthe enhancer sequence from the Banana Streak Virus strainAcuminata Vietnam fused to apromoter sequence fromDahliaMosaic Virus (DaMV).
  source 1..822: mol_type = other DNA
agacatcctggaccaatatgctgaagattatgctacctacaccaggataggacttgaagcacttaaccttgaagattggttcgaagaaccagaacccgatccacctaaccctgtggaccgccagaggatagaggacatcctggacctactgaacgtcagcaatgacgactgaaagattcccaggacaccggcggaagtggtggacccagtctaggtgcgatgcttagtcgcgcacgatgactatgtcggaaggcatctttgctttcggcaaactttagtaatactttaaggaaagtattgtacaagttaggtgcagagacaataatgcacccagctttagctttgtttatggaattattgtgtcggttgcattattggatgcctgcgtgcaccctaagcaatcaacggagaaacaaagataaaaatcaattactcacatgaaagagtattgatcacgagtcactatggagcgacaatctccagacaggatgtcagcatcttatcttcctttgaagaaagcatcatcaataacgatgtaatggtggggacatccactaagttattgctctgcaaacagctcaaaaagctactggccgacaatcataattgctcggcatgtgcaggtggggcctccactagcaataatacaagctttacagcttgcagtgactcatcctccaataatggagaaaaagacgtcagcagtgacgaacaagggtcgaaagacttgcctatataagggcattctcccctcagttgaagatcatcgaaagttggagcaataaactctctcttcaacaaatctatcttttatcttttatc

SEQ ID NO: 38 - DNA, 3681 bp, synthetic construct
  misc_feature 1..3681: note = Synthetic polynucleotide.Codon optimized LbCpf1(TYC) -CO2
  source 1..3681: mol_type = other DNA
tcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgcgcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgagggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcat

SEQ ID NO: 39 - protein, 1227 aa, synthetic construct
  REGION 1..1227: note = Synthetic polypeptide.LbCpf1(TYC) variant comprisingG532R andK595R substitutions
SKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 40 - DNA, 6080 bp, synthetic construct
  misc_feature 1..6080: note = Synthetic polynucleotide.Expression cassette comprising Zea maysUbiquitin promoter cassette, NLS-LbCpf1(TYC)-CO2-NLS and an Oryzasativa transcription termination sequence
  source 1..6080: mol_type = other DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttctgcaggtccggtacctcagcatgggtagcaaaaagaggcgtatcaagcaggacgcgtcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgcgcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgagggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcatgcaggatctaagaagcgtaggatcaagcaagattaagaggttaattaatcgatcctccgatcccttaattaccataccattacaccatgcatcaatatccatatatatataaaccctttcgcacgtacttatactatgttttgtcatacatatatatgtgtcgaacgatcgatctatcactgatatgatatgattgatccatcagcctgatctctgtatcttgttatttgtataccgtcaaataaaagtttcttccacttgtgttaataattagctactctcatctcatgaaccctatatataactagtttaatttgctgtcaattgaacatgatgatcgatg

SEQ ID NO: 41 - DNA, 3753 bp, synthetic construct
  misc_feature 1..3753: note = Synthetic polynucleotide.NLS-LbCpf1(TYC)-CO2-NLS
  source 1..3753: mol_type = other DNA
atgggtagcaaaaagaggcgtatcaagcaggacgcgtcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgcgcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgagggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcatgcaggatctaagaagcgtaggatcaagcaagattaa

SEQ ID NO: 42 - DNA, 33 bp, Solanum tuberosum
  source 1..33: mol_type = unassigned DNA
atgggtagcaaaaagaggcgtatcaagcaggac

SEQ ID NO: 43 - protein, 1300 aa, Francisella tularensis
  source 1..1300: note = subsp. novicida U112
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 44 - DNA, 3897 bp, synthetic construct
  misc_feature 1..3897: note = Synthetic polynucleotide.Codon optimized FnCpf1-Hs
  source 1..3897: mol_type = other DNA
agcatctaccaggagttcgtcaacaagtattcactgagtaagacactgcggttcgagctgatcccacagggcaagacactggagaacatcaaggcccgaggcctgattctggacgatgagaagcgggcaaaagactataagaaagccaagcagatcattgataaataccaccagttctttatcgaggaaattctgagctccgtgtgcatcagtgaggatctgctgcagaattactcagacgtgtacttcaagctgaagaagagcgacgatgacaacctgcagaaggacttcaagtccgccaaggacaccatcaagaaacagattagcgagtacatcaaggactccgaaaagtttaaaaatctgttcaaccagaatctgatcgatgctaagaaaggccaggagtccgacctgatcctgtggctgaaacagtctaaggacaatgggattgaactgttcaaggctaactccgatatcactgatattgacgaggcactggaaatcatcaagagcttcaagggatggaccacatactttaaaggcttccacgagaaccgcaagaacgtgtactccagcaacgacattcctacctccatcatctaccgaatcgtcgatgacaatctgccaaagttcctggagaacaaggccaaatatgaatctctgaaggacaaagctcccgaggcaattaattacgaacagatcaagaaagatctggctgaggaactgacattcgatatcgactataagactagcgaggtgaaccagagggtcttttccctggacgaggtgtttgaaatcgccaatttcaacaattacctgaaccagtccggcattactaaattcaataccatcattggcgggaagtttgtgaacggggagaataccaagcgcaagggaattaacgaatacatcaatctgtatagccagcagatcaacgacaaaactctgaagaaatacaagatgtctgtgctgttcaaacagatcctgagtgataccgagtccaagtcttttgtcattgataaactggaagatgactcagacgtggtcactaccatgcagagcttttatgagcagatcgccgctttcaagacagtggaggaaaaatctattaaggaaactctgagtctgctgttcgatgacctgaaagcccagaagctggacctgagtaagatctacttcaaaaacgataagagtctgacagacctgtcacagcaggtgtttgatgactattccgtgattgggaccgccgtcctggagtacattacacagcagatcgctccaaagaacctggataatccctctaagaaagagcaggaactgatcgctaagaaaaccgagaaggcaaaatatctgagtctggaaacaattaagctggcactggaggagttcaacaagcacagggatattgacaaacagtgccgctttgaggaaatcctggccaacttcgcagccatccccatgatttttgatgagatcgcccagaacaaagacaatctggctcagatcagtattaagtaccagaaccagggcaagaaagacctgctgcaggcttcagcagaagatgacgtgaaagccatcaaggatctgctggaccagaccaacaatctgctgcacaagctgaaaatcttccatattagtcagtcagaggataaggctaatatcctggataaagacgaacacttctacctggtgttcgaggaatgttacttcgagctggcaaacattgtccccctgtataacaagattaggaactacatcacacagaagccttactctgacgagaagtttaaactgaacttcgaaaatagtaccctggccaacgggtgggataagaacaaggagcctgacaacacagctatcctgttcatcaaggatgacaagtactatctgggagtgatgaataagaaaaacaataagatcttcgatgacaaagccattaaggagaacaaaggggaaggatacaagaaaatcgtgtataagctgctgcccggcgcaaataagatgctgcctaaggtgttcttcagcgccaagagtatcaaattctacaacccatccgaggacatcctgcggattagaaatcactcaacacatactaagaacgggagcccccagaagggatatgagaaatttgagttcaacatcgaggattgcaggaagtttattgacttctacaagcagagcatctccaaacaccctgaatggaaggattttggcttccggttttccgacacacagagatataactctatcgacgagttctaccgcgaggtggaaaatcaggggtataagctgacttttgagaacatttctgaaagttacatcgacagcgtggtcaatcagggaaagctgtacctgttccagatctataacaaagatttttcagcatacagcaagggcagaccaaacctgcatacactgtactggaaggccctgttcgatgagaggaatctgcaggacgtggtctataaactgaacggagaggccgaactgttttaccggaagcagtctattcctaagaaaatcactcacccagctaaggaggccatcgctaacaagaacaaggacaatcctaagaaagagagcgtgttcgaatacgatctgattaaggacaagcggttcaccgaagataagttctttttccattgtccaatcaccattaacttcaagtcaagcggcgctaacaagttcaacgacgagatcaatctgctgctgaaggaaaaagcaaacgatgtgcacatcctgagcattgaccgaggagagcggcatctggcctactataccctggtggatggcaaagggaatatcattaagcaggatacattcaacatcattggcaatgaccggatgaaaaccaactaccacgataaactggctgcaatcgagaaggatagagactcagctaggaaggactggaagaaaatcaacaacattaaggagatgaaggaaggctatctgagccaggtggtccatgagattgcaaagctggtcatcgaatacaatgccattgtggtgttcgaggatctgaacttcggctttaagagggggcgctttaaggtggaaaaacaggtctatcagaagctggagaaaatgctgatcgaaaagctgaattacctggtgtttaaagataacgagttcgacaagaccggaggcgtcctgagagcctaccagctgacagctccctttgaaactttcaagaaaatgggaaaacagacaggcatcatctactatgtgccagccggattcacttccaagatctgccccgtgaccggctttgtcaaccagctgtaccctaaatatgagtcagtgagcaagtcccaggaatttttcagcaagttcgataagatctgttataatctggacaaggggtacttcgagttttccttcgattacaagaacttcggcgacaaggccgctaaggggaaatggaccattgcctccttcggatctcgcctgatcaactttcgaaattccgataaaaaccacaattgggacactagggaggtgtacccaaccaaggagctggaaaagctgctgaaagactactctatcgagtatggacatggcgaatgcatcaaggcagccatctgtggcgagagtgataagaaatttttcgccaagctgacctcagtgctgaatacaatcctgcagatgcggaactcaaagaccgggacagaactggactatctgattagccccgtggctgatgtcaacggaaacttcttcgacagcagacaggcacccaaaaatatgcctcaggatgcagacgccaacggggcctaccacatcgggctgaagggactgatgctgctgggccggatcaagaacaatcaggaggggaagaagctgaacctggtcattaagaacgaggaatacttcgagtttgtccagaatagaaataac

SEQ ID NO: 45 - DNA, 3900 bp, synthetic construct
  misc_feature 1..3900: note = Synthetic polynucleotide.Codon optimized FnCpf1-CO1
  source 1..3900: mol_type = other DNA
atgtctatctatcaagagtttgtcaacaagtacagcttaagtaagacacttcgcttcgagttgatccctcaggggaagacactggaaaacatcaaggcgaggggccttatcctggacgacgagaagcgggctaaagactacaaaaaagctaaacagataattgacaagtatcaccaattttttattgaggagatcctctcctctgtgtgcataagtgaggatctgctccagaactattcggatgtttactttaaactcaagaagtccgatgatgacaaccttcagaaggacttcaagtccgccaaagacacgattaagaaacaaatcagtgagtacatcaaggatagcgagaaattcaagaacctgttcaaccagaacttaattgatgccaaaaagggccaagaatccgacctcatcctgtggttaaagcaatctaaagacaacggtattgagctgttcaaggcaaacagcgacattacagacattgacgaggccctagagatcatcaagtcattcaaaggctggacgacttactttaaaggttttcacgagaaccgtaagaatgtttactcaagtaacgatataccaacgagcattatctaccgaatagtggatgataacctaccgaagttccttgagaacaaagcgaagtacgagtctctcaaagacaaggcccctgaggccatcaactacgagcagattaagaaggatctcgccgaggagctaaccttcgacattgactacaaaacatcggaagtgaatcagagggtgttctcgcttgatgaagtattcgagattgctaacttcaacaattacctgaaccagagtggtattactaagttcaacacaatcattggaggcaaattcgtgaacggcgaaaacacaaagcgaaaagggataaacgagtacattaacttgtacagccagcagatcaacgataagacactcaagaagtataagatgtctgtgctgttcaaacaaatcttaagcgacacggaaagcaagtcgttcgtaattgacaagctggaagacgattctgacgtggttacaaccatgcagtccttttacgagcagattgccgcattcaagaccgtggaggaaaagtcgatcaaggaaacactttcgttgcttttcgacgaccttaaagctcagaagctcgacttaagcaagatatactttaagaacgataagagcttgacagacttgagccagcaagtctttgacgactacagcgttatcggaactgccgttctggagtacataacacagcagatcgcacccaagaaccttgacaacccttccaagaaagaacaagagttgatcgccaagaagactgaaaaggctaagtacctctctctggagactatcaagctcgctcttgaggagtttaacaagcacagggacattgacaagcaatgccgattcgaggaaatactggcaaacttcgcagccatacccatgatattcgatgagatagcccagaacaaggataacttggcccaaatctcgattaagtatcagaaccagggcaaaaaggaccttctacaggctagtgcagaggacgatgtgaaggctattaaggacttattagatcagacaaacaaccttctgcataagctcaagatattccatatctcccagtcagaggacaaggccaacattctggataaggacgagcacttctatctcgtattcgaggaatgttactttgagctggccaatatcgttcccttgtacaacaaaatccggaactacatcacacagaagccctacagtgatgagaagttcaaattgaactttgaaaactcaacacttgctaatggttgggacaagaataaggaacctgacaacactgccatcctctttattaaagatgataagtactacctcggggtgatgaataagaagaacaacaaaatcttcgatgacaaagctattaaggagaacaaaggtgaagggtacaagaagattgtctacaaactgctgcctggtgccaacaaaatgctaccaaaggtatttttcagcgccaaatctattaagttctacaatccaagcgaggatattctccggatacggaatcactctacacataccaagaatggaagtccacaaaagggttacgagaaattcgagttcaacattgaagactgccggaaattcattgacttctacaagcaatccatctctaaacatcctgaatggaaagacttcggtttccgcttcagtgatactcaacggtacaattcaattgacgaattctaccgtgaggttgagaaccaggggtacaaactgaccttcgagaacatatcagagagctacatcgactcagtggttaatcaggggaagctatatctgtttcaaatctacaacaaagactttagtgcctactctaaagggcggccaaacttacacacactttactggaaggcactattcgacgaacgcaatctacaagatgtagtttacaaattgaacggtgaggctgagttgttctaccgtaaacaatctatacccaagaagataacacaccctgctaaagaggcaattgcaaacaaaaacaaggataatcccaaaaaggagtctgtctttgagtatgacctcattaaggataagcggttcacggaggacaagttcttcttccattgtccaataaccatcaacttcaaatcatccggcgcaaacaaattcaatgacgagatcaacctgttactaaaggagaaggctaacgatgttcacatcttatctattgatcgaggtgagagacacctagcctactacactttagtggatgggaaggggaacatcatcaagcaagacaccttcaacatcattgggaacgacaggatgaagactaactaccatgataagctcgccgctattgaaaaggacagggactctgccaggaaggactggaaaaaaattaacaatattaaagagatgaaggagggctacctgagccaagtagtccatgagatagcaaaactggtgattgagtacaacgcaatagtcgtattcgaggacttaaacttcggcttcaaacgtgggcggtttaaggtggagaaacaagtctatcagaaattggagaagatgctaatcgagaagctcaactacctcgtgtttaaagacaacgagtttgacaaaactggaggagtcctgcgggcataccaactgaccgcacccttcgagacattcaagaagatgggaaagcagactggcatcatctattacgtgccagcgggttttacttccaaaatctgtccagttacaggcttcgtgaaccagttgtacccgaagtacgagtctgtttccaagtcacaggaattcttctcaaagtttgacaagatatgttacaatctcgataagggatactttgagtttagtttcgactacaagaactttggcgataaggccgcaaaagggaaatggacaattgcatccttcgggtcacgccttattaactttcgtaactcagacaagaaccacaattgggacaccagggaggtgtaccctactaaggagctggagaagctacttaaagactactcgattgagtacggacatggagagtgcatcaaggcagcaatatgtggggaatctgacaaaaagttctttgccaagctgacctctgtactgaacactattctccaaatgagaaatagtaagactggcacagagttggactacctgatctctccagtggctgacgttaatgggaattttttcgactcaagacaagctcccaagaatatgccacaggacgcagatgcaaacggggcatatcacatcgggcttaaaggactcatgctactagggcggatcaagaataatcaggagggcaaaaagctgaacctagtcatcaagaacgaggagtacttcgaatttgtccagaatcgtaacaac

SEQ ID NO: 46 - DNA, 3900 bp, synthetic construct
  misc_feature 1..3900: note = Synthetic polynucleotide.Codon optimized FnCpf1-CO2
  source 1..3900: mol_type = other DNA
atgtccatataccaggagttcgtcaacaagtactcgctcagcaagacgctccgcttcgagctgatcccccagggtaagaccctggagaacatcaaggcgcgcggactcatcctcgacgacgagaagcgggctaaggactacaaaaaggccaagcagatcatcgacaagtaccaccagttcttcatcgaggagatcctctcctccgtctgcatctccgaggacctcctccagaactacagcgacgtctacttcaagctcaaaaagtcggacgacgacaacctccagaaggacttcaagtcggcgaaggacaccatcaagaagcagatcagcgagtacatcaaggactccgagaagttcaagaacctgttcaaccagaacctgatcgacgccaagaagggccaggagtcggacctgatcctgtggctcaagcagagcaaggacaacggcatcgagctcttcaaggccaacagcgacatcacggacatcgacgaggccctggagatcatcaagtcgttcaagggctggacgacctacttcaagggcttccacgagaaccgcaagaacgtctatagctccaacgacatccccacctcgatcatctaccggatcgtggacgacaacctccccaagttcctggagaacaaggcgaagtacgagtcgttgaaggacaaggcgccggaggcgatcaactacgagcagatcaagaaggacctggccgaggagctgaccttcgacatcgactacaagacgtcggaggtgaaccagcgggtgttcagtctggacgaggtcttcgagatcgcgaacttcaacaactacctgaaccagtcggggatcaccaagttcaacacgatcataggcggcaagttcgtgaacggcgagaacaccaagcgcaaggggataaacgagtacatcaacctgtacagccagcagatcaacgacaagacgctcaagaagtacaagatgagcgtgctcttcaagcagatcctcagtgacaccgagtccaagagcttcgtgatcgacaagctggaggacgacagcgacgtcgtgacaaccatgcagagcttctacgagcagatcgccgcgttcaagaccgtcgaggagaagtcaatcaaggagacgctctccttgctgttcgacgacctgaaggcacaaaagctggacctcagcaagatctacttcaagaacgacaagtccctgaccgacctgagccagcaggttttcgacgactactccgtcatcgggactgccgtcctggagtacatcacccagcagatcgctccgaagaacctcgacaacccgtccaagaaggagcaggagctgattgcgaagaagacagagaaggccaagtacctctccctcgagaccatcaagctcgccctggaggagttcaacaagcacagggacattgacaagcagtgccgtttcgaggagatcctggccaacttcgcggccatccccatgatcttcgacgagatcgcccagaacaaggacaacctggcgcagatctctatcaagtaccagaaccaggggaagaaggacttgctccaggcctcagcggaggacgacgtgaaggccatcaaggacctgctcgaccagacgaacaacttgctccacaagctcaagatctttcacatcagccagagcgaggacaaggccaacatcctcgacaaggacgagcacttctacctcgtgttcgaggagtgctacttcgagctggccaacatcgtgccactttacaacaagatccgcaactacatcacgcagaagccgtactccgacgagaagttcaagctgaacttcgagaactccaccctcgccaacggctgggacaagaacaaggagccggacaacaccgcgatcctgttcataaaggacgacaagtactacttgggggtcatgaacaagaagaacaacaagatattcgacgacaaggccatcaaggagaacaagggggagggctacaagaagatcgtctacaagctcctccccggcgcgaacaagatgctgcctaaggtcttcttttccgccaagagtatcaagttctacaacccctccgaggacatcctccgcatccggaaccacagcacgcacacaaagaacggctcgcctcagaagggctacgagaagttcgagttcaacatcgaggactgccggaagttcatcgacttctacaagcagagcatctccaagcacccggagtggaaggactttggcttcaggttctcagacacccagcggtacaactccatcgacgagttctaccgcgaggtggagaaccagggctacaagctgaccttcgagaatatatcagagtcgtacatcgacagcgtggtgaaccagggcaagttgtacctgttccagatctacaacaaggacttctccgcctactcaaaggggcgtccaaacctgcacacgctgtactggaaggcgctcttcgacgagcgcaacctacaagatgttgtatacaagctcaacggcgaggcggaactgttctataggaagcagtcgatccccaagaagattacgcacccggctaaggaggccatcgccaacaagaacaaggacaaccccaagaaggagtccgtgttcgagtacgacctcatcaaggacaagaggttcacggaggacaagtttttcttccactgcccaatcactatcaatttcaagtcgagcggagccaacaagttcaacgacgagataaacctgctcctcaaggagaaggccaatgacgtgcacatcctctccatcgaccggggcgagcggcacctggcgtactacacgctggtggacggcaagggcaacatcatcaagcaggacaccttcaacatcatcgggaacgaccgcatgaagaccaactaccacgacaagctcgccgccatcgagaaggacagggactccgcgcgcaaggactggaagaagattaacaacatcaaggagatgaaggagggctacctcagccaggtggtccacgagatcgccaagctcgtcattgagtacaacgccatcgtcgtcttcgaggacctgaatttcggcttcaagcgcggccggttcaaggtggagaagcaggtctaccagaagcttgagaagatgctgatcgagaagctgaactacctggtgttcaaggacaacgagttcgacaagaccggcggagtgctgcgcgcctaccagctcacggcgcctttcgagacgttcaagaagatgggcaagcagacgggcatcatctactacgtgcccgccggcttcacctctaagatctgcccagtgaccggcttcgttaaccagctgtacccgaagtacgagagcgtgtccaagtcccaggagttcttctccaagttcgacaagatttgttacaacctcgacaagggctacttcgagttttcgttcgactacaagaactttggcgacaaggcggccaaggggaagtggaccatcgcctctttcggcagcaggctcatcaatttccggaactccgacaagaaccacaactgggacacgcgcgaggtgtacccgacgaaggagctggagaagctgctcaaggactactccatcgagtacggccacggcgagtgcatcaaggcggcgatctgcggggagagcgacaagaagttctttgccaagctgaccagcgtgctgaacaccatcctccagatgcggaactccaagaccggcaccgagctggactacctgatctccccggtcgcggacgtcaacgggaacttcttcgactcccgacaggctcccaagaacatgccccaggacgccgacgcgaacggcgcgtaccacatcggcctcaagggcctgatgctgctggggcgcatcaagaacaaccaggagggcaagaagctgaacctcgtgatcaagaacgaggaatacttcgagttcgtgcagaaccgcaacaac

SEQ ID NO: 47 - DNA, 3900 bp, synthetic construct
  misc_feature 1..3900: note = Synthetic polynucleotide.Codon optimized FnCpf1-CO3
  source 1..3900: mol_type = other DNA
atgagcatctaccaggagttcgtgaacaagtacagcctgtcgaagaccctccggttcgagctgatccctcaggggaagacgctggagaacatcaaggcgcgcggcctcatcctcgacgacgagaagcgggcgaaggactacaaaaaggcaaagcagatcatcgacaagtaccaccaattctttattgaggagatcctcagctccgtctgcatcagcgaggacttgctccagaactactccgacgtctatttcaagctcaagaagtcagacgacgacaacctccagaaggacttcaagtccgcgaaggacacgatcaaaaagcagatcagcgagtacatcaaggactccgagaagttcaagaacctcttcaaccagaacctgatcgacgccaagaagggccgggaatcggacctcatcctgtggctcaagcagtcgaaggacaacggcatcgagctgttcaaggccaactccgacatcaccgacatcgacgaggcgctggagatcatcaagtcgttcaaggggtggaccacctacttcaagggcttccacgagaaccgcaagaacgtttactccagcaacgacatccccacctcgatcatctaccggatcgtggacgacaacctgcccaagttcctggagaacaaggccaagtacgagtccctcaaggacaaggccccggaggcgatcaactacgagcagattaaaaaggaccttgctgaggagctgaccttcgacatcgactacaagacctccgaggtgaaccagcgggtgttcagcctcgacgaggtgttcgagatcgccaacttcaacaactacctgaaccagtccgggatcaccaagttcaacacgatcatcggcgggaagttcgtcaacggcgagaacacgaagcggaagggcatcaacgagtacatcaacctctacagccagcagatcaacgacaagaccctcaaaaaatacaaaatgtcggtcctgttcaagcagatcctgtccgacaccgagtccaagagcttcgtcatcgacaagctggaggacgactccgacgtcgtgaccaccatgcagtccttctacgagcagatcgccgccttcaagaccgtggaggagaagtccatcaaggagaccctcagcctcctcttcgacgacctcaaggcgcagaagctggacctctccaagatatacttcaagaacgacaagagcctcaccgacctgtcccagcaagtattcgacgactacagcgtgatcgggacggcggtgctggagtacatcacccagcagatagcgcccaagaacctggacaacccctccaagaaagaacaggagctgattgctaaaaagaccgaaaaggctaagtacctgtccctggagaccatcaagctcgcgctggaggagttcaacaagcaccgggacatcgacaagcagtgccggttcgaggagatcctagcaaacttcgccgcgatccccatgatcttcgacgagatcgcccagaacaaggacaacctggcccagatcagcatcaagtaccagaaccagggcaagaaggaccttcttcaagctagtgccgaggacgacgtgaaggcgattaaggatctgctcgaccagaccaacaacctgctccacaagctcaagatattccacatctcccagtccgaggacaaggccaacatcctggacaaggacgagcacttctacctggtgttcgaggagtgctacttcgagctggccaacatcgtgccgctgtacaacaagatccggaactacatcacccagaagccctactccgacgagaagttcaagctgaacttcgagaactccaccctggcgaacgggtgggacaagaacaaggagcccgacaacacggccatcctcttcatcaaggacgacaaatattatctgggcgtcatgaacaaaaagaacaacaagatattcgatgacaaggcgatcaaggagaacaagggcgagggctacaagaaaatagtatataaactactgcccggcgcgaacaagatgctcccgaaggtgttttttagtgcaaagtctattaagttctacaaccccagcgaggacatcctccgcatccggaaccacagcacgcacaccaagaacggcagcccacagaagggctacgagaagttcgagttcaacatcgaggactgccgcaagttcatcgacttctacaagcagtccatctccaagcaccccgagtggaaggacttcgggttccggttcagcgacacccagcgctacaacagcatcgacgagttctaccgggaggtcgagaaccaggggtacaagctgacgttcgagaacatctccgagagctacatcgacagcgtggtgaaccaggggaagctgtacctgtttcagatatacaacaaggacttctcagcctacagcaaggggcggccgaacctgcacaccctgtactggaaggccctgttcgacgagcggaacctccaggacgtcgtgtacaagctcaacggcgaggcggagctgttctaccggaagcagtccatccccaaaaagattactcaccccgcgaaggaggccatcgccaacaagaacaaggacaaccccaaaaaggaatcagtgttcgagtacgacctcatcaaggacaagcgcttcaccgaggacaaattcttctttcactgcccgatcacgatcaacttcaagtcctccggggcgaacaagttcaacgacgagatcaacctgctgctcaaggagaaggccaacgacgtgcacatcctcagcatcgaccggggcgagcgccacctggcctactacaccctggtggacgggaagggcaacatcataaagcaagataccttcaacatcatcgggaacgaccggatgaagacgaactaccacgacaagctggcggccatcgagaaggaccgggacagcgcccgcaaggactggaaaaagataaacaacattaaggagatgaaggagggctacctgtcccaggtggtccacgagatcgccaagctcgtcatcgagtacaacgccatcgtcgtgttcgaggacttgaacttcgggttcaagcggggccggttcaaggtggagaaacaagtctatcaaaagctggagaagatgctcatcgagaagctcaactacctcgtgttcaaggacaacgagttcgacaagaccggcggcgtcctgcgggcctaccagctcaccgcgccgttcgagacgttcaagaagatggggaagcagacggggatcatctactacgtccccgccgggttcaccagcaagatatgcccggtcacggggttcgtcaaccagctctaccccaagtacgagtcggtgagcaagagccaggagttcttcagcaagttcgacaagatctgctacaacctggacaagggctacttcgagttctcgttcgactacaagaacttcggggacaaggcggcgaagggcaagtggaccatcgccagcttcggctcccgcctcatcaacttccggaactcggacaagaaccacaactgggacacccgcgaggtgtaccccacgaaggagctggagaagctgctcaaggactacagcatcgagtacgggcacggcgagtgcatcaaggccgccatctgcggggagtccgacaagaaattctttgccaagctgacctccgtgctgaacaccatcctccagatgcggaacagcaagaccgggaccgagctggactacctgatctccccggtcgccgacgtcaacggcaacttcttcgattctcgccaggctcccaagaacatgccccaggacgccgacgccaacggggcctaccacatcgggctcaagggcctcatgctgctcgggcggatcaagaacaaccaggagggcaagaaactcaacctggtcatcaagaacgaggagtactttgagttcgtccagaaccggaacaac

SEQ ID NO: 48 - DNA, 3900 bp, synthetic construct
  misc_feature 1..3900: note = Synthetic polynucleotide.Codon optimized FnCpf1-CO4
  source 1..3900: mol_type = other DNA
atgagcatctaccaggagttcgtgaacaagtacagcctgagcaagaccctgcgcttcgagctgattcctcaggggaagaccctggagaacatcaaggcgcgcggcctgatcctggacgacgagaagcgggcgaaggactacaaaaaggcgaagcagatcatcgacaagtaccatcagtttttcatcgaggagattctcagctccgtgtgcatcagcgaagacctgctccagaactacagcgacgtttacttcaagctcaagaaatcggacgacgacaacctccagaaggacttcaagagcgcgaaggacacgattaagaaacagatcagcgagtacatcaaggactccgagaagttcaagaacctgttcaaccagaacctcatcgacgccaagaaaggccaagagtcggacctcatcctctggctcaagcagagcaaggacaacggcatcgagctgttcaaggcgaacagcgacatcaccgacatcgacgaggcgctggagatcatcaagtcgttcaagggctggacgacctacttcaagggcttccacgagaaccgcaagaatgtctactcgagcaacgacatccccacgtcgatcatctaccgcatcgtggacgacaacctcccgaagttcctggagaacaaggcgaagtacgagagcctcaaggacaaggccccggaggccatcaactacgagcagatcaaaaaggatttggctgaggagctgacgttcgacatcgactacaagacctccgaggtgaaccagcgcgtcttcagcctcgacgaggtgttcgagatcgccaacttcaacaactacctcaaccagtcgggcatcaccaagttcaacaccatcatcggcgggaagttcgtcaacggcgagaacacgaagcgcaaggggatcaacgagtacatcaacctgtacagccagcagatcaacgacaagaccctcaagaagtataaaatgtcggtgctgttcaagcagatcctctcggacaccgagagcaagtcgttcgtcatcgacaagctggaggacgacagcgacgtggtgaccaccatgcagagcttctacgagcagatcgcggccttcaagaccgtcgaggagaagtcgatcaaggagacgctcagcctgctgttcgacgacctcaaggcccagaagctcgacctgtccaagatatactttaagaacgacaagagcctcacggacctgtcccagcaggtattcgacgactacagcgtgatcgggacggccgtgctggagtacatcacccaacagatcgcgcccaagaacctggacaacccgtccaagaaggaacaagagctaatcgccaaaaagactgagaaggcgaagtacctgtcgctggagacgatcaagctcgcgcttgaggagtttaacaagcaccgcgacatcgacaagcagtgccggttcgaggagatcctggccaacttcgcggcgatcccgatgatcttcgacgagatcgcccagaacaaggacaacctggcgcagatcagcatcaagtaccagaaccagggcaaaaaagacttgctccaagctagtgcggaggacgacgtgaaggcgattaaggatctgctggaccagactaacaatctgctgcacaagctcaagatctttcacatctctcagtcggaggacaaggcgaacatcctggacaaggacgagcacttctacctagtgttcgaggagtgctacttcgagctggcgaacatcgtgcccctgtacaacaagatccggaactacatcacccagaagccctacagcgacgagaagttcaagctgaacttcgagaacagcacgctggcgaacgggtgggacaagaacaaggagcccgacaacaccgccatcctgttcatcaaggacgacaaatattacctcggcgtcatgaacaaaaagaataacaagatattcgatgacaaggcgatcaaggagaacaagggcgagggctacaagaagatcgtatataaactcctgccgggagcgaacaagatgctcccgaaggttttctttagtgccaagtccatcaagttctacaaccccagcgaggacatcctccgcatccggaaccactccacccacaccaagaacggctcgccgcagaagggctacgagaagttcgagttcaacatcgaggactgccgcaagttcatcgacttctacaagcagtccatctccaagcacccggagtggaaggacttcgggttccggttctccgacacgcagcgctacaactccatcgacgagttctaccgggaggtggagaaccagggctacaagctgacgttcgagaacatctcggagtcctacatcgactccgtggtcaaccagggcaagctgtacctcttccagatatacaataaggacttctccgcctacagcaaggggcggcccaacctccacaccctgtactggaaggcgctcttcgacgagcggaacctccaggacgtcgtgtacaagctgaacggcgaggcggagctgttctaccgcaagcagagcatccccaagaagatcacgcaccccgcgaaggaggccatcgccaacaagaacaaggacaaccccaagaaggaatcggtcttcgagtacgacctcatcaaggacaagcggttcacggaggacaaattctttttccactgcccgatcactattaacttcaagtccagcggcgcgaacaagttcaacgacgagatcaacctgctcctcaaggagaaggcgaacgacgtgcacatcctcagcatcgaccggggcgagcgccacctcgcctactacacgctggtggacgggaagggcaacatcatcaagcaagacaccttcaacatcatcggcaacgaccggatgaagaccaactaccacgacaagctggccgccatcgagaaggaccgcgactcggcccgcaaggactggaaaaagatcaacaatatcaaggagatgaaggagggctacctgagccaagttgtccacgagatcgccaagctggtgatcgagtacaacgccatcgtcgtgttcgaagacctgaacttcggcttcaagcgcggccggttcaaggtcgagaaacaagtctatcagaaacttgagaagatgctgatcgagaagctgaactacctcgtcttcaaggacaacgagttcgacaagaccggcggcgtcctccgcgcgtaccagctcaccgcgccgttcgagacgttcaagaaaatgggcaagcagaccggcatcatctactacgtgcccgccgggttcacgagcaaaatatgtcccgtgaccggcttcgtcaaccagctctaccccaagtacgagtccgtgtcgaagtcccaggaattcttcagcaagttcgacaagatatgctacaacctggacaagggctacttcgagttctccttcgactacaagaacttcggggacaaggcggcgaaggggaagtggaccatcgcctcgttcgggtcgcgcctcatcaacttccggaacagcgacaagaaccacaactgggacacccgcgaggtgtacccgacgaaggagctggagaagctcctcaaggactacagcatcgagtacggccacggggagtgcatcaaggcggccatctgcggcgagtcggacaaaaagttctttgccaaactcacctcggtcctcaacaccatcctccagatgcggaacagcaagacgggcacggagctggactacctcatcagcccggtggccgacgtgaacggcaatttctttgactcacgccaggcccctaagaacatgccccaggacgccgacgccaacggcgcgtaccacatcggcctcaagggcctgatgctgctcggccggatcaagaacaaccaggagggcaagaagctcaacctggtcatcaagaacgaggagtatttcgagttcgtccagaaccgcaacaac

SEQ ID NO: 49 - DNA, 3900 bp, synthetic construct
  misc_feature 1..3900: note = Synthetic polynucleotide.Codon optimized FnCpf1-CO5
  source 1..3900: mol_type = other DNA
atgtccatataccaggagtttgttaataaatatagcttgtccaaaaccctgcggtttgaactcatacctcaggggaagactttggagaatatcaaagcgcggggactgatactggacgacgaaaagcgcgcaaaagattacaagaaagcgaaacagatcatcgataaataccatcaatttttcatagaggagattctcagttctgtctgtatcagtgaggacctcctccaaaattattcagacgtctattttaaactcaagaagtcggacgacgacaaccttcagaaagattttaagtcagcaaaagacacaatcaaaaaacaaatatcggaatacataaaggactcagaaaagttcaagaatctttttaaccaaaatctgatagacgcgaagaaagggcaggaatctgatcttatactctggcttaagcagtctaaagacaacggcatagaactctttaaggcaaacagcgatataaccgacatagatgaagccctcgagataattaagtccttcaaaggctggactacatattttaaagggttccatgagaataggaagaacgtgtattcctcgaatgatattcccacctcgataatctaccggattgtggatgataatctgcctaaatttttggaaaataaagcgaagtacgaaagtttgaaagataaagcaccagaagcaattaattatgaacaaattaagaaagatctggctgaggaacttacgttcgatatcgattataaaacatcagaagttaatcagcgggtttttagcctggatgaagtttttgagatcgccaacttcaacaattatcttaatcagagcgggattaccaaattcaacactattatcggtggtaaattcgttaatggtgagaacacaaagagaaaaggtataaacgaatacataaatttgtacagtcaacagattaacgataaaactttgaaaaagtacaagatgtcagtgcttttcaaacaaatcctttccgacacagaatccaaaagttttgtgatagacaaattggaagatgatagcgacgtcgtcacgaccatgcaatcattttatgagcaaattgcagccttcaagacggttgaggaaaaaagtataaaagaaacgttgtcgctcttgttcgacgacctgaaagcacagaaattggatttgtctaagatatactttaaaaacgacaaatccctcacggacttgtcgcagcaagtctttgatgattattcggtgattgggacagccgtgctcgaatacatcacccagcaaatcgctccgaaaaatctggacaatccgtctaaaaaagagcaagagctgatcgcaaagaaaaccgaaaaagcgaaatacctgtctctggagacaatcaaattggccttggaagagttcaataagcacagagacattgataaacaatgtcggtttgaggaaattcttgctaactttgccgctatcccgatgatcttcgatgagattgcgcaaaataaggataatctggcccaaatctcgatcaagtatcaaaatcagggtaagaaggacctgcttcaagcatcggcggaggatgatgtgaaagcgattaaggacttgttggatcagaccaacaatttgttgcacaagctgaagatattccacatctcccagagtgaggataaggccaacatcctggacaaggatgaacatttctatttggtcttcgaagagtgttattttgaattggccaacatagttcctctttataacaagatccgcaattatattacacaaaagccttattccgatgaaaaatttaaacttaacttcgaaaatagcacattggcgaatggttgggataaaaataaggagcctgacaatactgctatacttttcattaaggacgataagtactacctcggcgttatgaacaagaagaataataagatctttgacgacaaagcaatcaaagagaacaaaggcgaaggttacaaaaaaatcgtgtacaaactcctgcctggcgcgaataaaatgcttccgaaggtttttttcagtgcgaagtccattaagttttataacccttccgaggatattttgagaattagaaatcactccacccataccaagaatggcagcccccagaaggggtatgaaaagttcgaatttaatatcgaagactgccgcaagtttatagacttttataaacagtccatatctaaacatcccgaatggaaagattttggtttccggttttctgacactcagaggtacaacagcatagatgagttctaccgcgaagttgaaaaccaaggctacaagcttacatttgagaacatcagcgagtcatatattgactcagtcgttaatcagggcaaactttatttgttccaaatttacaacaaagacttttcagcgtacagcaagggaaggccaaatctccatacactgtattggaaggcgctgtttgacgagcggaatcttcaagatgttgtgtataaactcaacggggaggccgaattgttctacaggaagcaaagcattcctaagaaaattacccaccccgctaaggaagcgatagcaaataaaaataaagacaatccgaaaaaagagagcgtttttgagtacgaccttataaaggataagagattcaccgaggataagtttttcttccactgtccaataactattaactttaaatcctccggagccaacaagtttaacgacgaaattaatttgctgctgaaagagaaggcgaacgacgttcacattctgtcaatagacagaggggagagacacttggcatactacacgctcgttgatggaaaaggtaacattattaagcaagatactttcaacatcattgggaatgacagaatgaaaacaaactatcacgataaactcgcggcaattgagaaggaccgggattcggcgaggaaagactggaagaaaatcaacaatataaaggagatgaaggaaggatacctgtctcaagtcgtccacgaaatagccaagcttgttatagagtataatgcgattgtggtctttgaagaccttaactttggatttaaacgcggccggtttaaagtcgagaaacaagtgtatcaaaaactggaaaaaatgttgatcgagaaactgaattatctcgtcttcaaggacaacgagttcgataaaaccgggggcgtcttgcgcgcttatcaactgacggcaccgtttgaaactttcaagaagatgggcaaacagactgggattatctactacgttcctgccggattcacctccaaaatatgcccagttactggatttgttaaccagttgtaccctaagtacgaatcagtcagcaagtcccaagagtttttctcaaaatttgataagatctgctataacctggacaaggggtacttcgagttttccttcgactataaaaatttcggcgacaaggcagctaaaggtaaatggacgattgcaagtttcggctccaggcttattaatttcagaaacagcgacaaaaaccataactgggacacgcgcgaggtctaccctacgaaggaactggaaaaacttctcaaggattacagtatagaatacggacacggggagtgtatcaaagctgcgatatgcggagagtcggataaaaagtttttcgcaaagttgacatcagttttgaacactatcttgcagatgagaaattccaagactggcacggagttggactaccttattagcccagtggcggatgtcaacgggaacttttttgattcgaggcaagcccctaagaatatgcctcaagacgctgatgcaaacggggcatatcacataggactcaaagggttgatgctgctgggcagaattaagaacaaccaggaaggaaagaagctcaatcttgttatcaaaaatgaagagtactttgaattcgttcaaaaccggaataac

SEQ ID NO: 50 - DNA, 3900 bp, synthetic construct
  misc_feature 1..3900: note = Synthetic polynucleotide.Codon optimized FnCpf1-CO6
  source 1..3900: mol_type = other DNA
atgagtatttatcaagagttcgttaataagtacagtctttctaagacgctcaggtttgagctcattccacaaggtaagacccttgagaatattaaagcccgcggactgatcctggacgacgagaagcgcgccaaagactataagaaggcgaaacagatcatagataagtatcaccaattcttcatagaagagatacttagctctgtttgtatctcagaggatctgctccagaactactcggatgtgtactttaaactcaagaagtctgacgatgacaatctccagaaggactttaagtccgccaaagacacaatcaagaagcagatttcggaatacataaaggacagtgagaagttcaagaacttgttcaaccagaatctcatagacgccaagaagggtcaggagagcgatcttattctgtggttgaaacaatcaaaggacaacggtattgagttgtttaaagcaaactctgacatcaccgacattgacgaggctctggagatcattaagagttttaaagggtggacaacctactttaagggatttcatgagaatcgcaagaacgtgtactcgagcaatgacatacctacaagcataatctatcggatagtcgacgataacctcccgaagttcctcgagaataaagcgaagtacgaatcgcttaaggataaggcgccggaggcgataaactatgaacagattaagaaggatctggctgaagaattgacctttgatattgactataagaccagcgaagtcaaccaacgcgttttcagcctggatgaagtgtttgagatcgccaacttcaataattacttgaatcaatcggggataacaaagtttaatacgattatcggcgggaagtttgtcaacggggagaacacaaagaggaagggcataaatgagtacattaatctctatagtcaacagataaatgacaagacgttgaagaaatacaagatgtcagtccttttcaagcagatcctgtctgacacagaatcgaagagcttcgtgatagataagctggaagatgattccgacgttgtcacaaccatgcaatcgttttacgagcagatcgcggccttcaagaccgttgaggagaagtctattaaagagactctttctcttctgtttgatgacttgaaggcccagaagcttgacttgtccaagatctacttcaagaacgataaatctctgacagatctcagccagcaagtgtttgacgactattctgttatcggaacggccgtgctggagtacatcacacagcagatcgcgcctaagaaccttgataatccgagcaagaaagaacaggagttgattgcaaagaagacagagaaggccaaatacctttctctggagacaattaaacttgcactggaagaattcaataagcacagagatatcgacaagcagtgtcgctttgaggagatcctggcaaattttgctgccatcccaatgatttttgatgagattgcccagaacaaggacaatctcgcgcagatatcaatcaagtatcagaaccaaggcaagaaggacctcctgcaagcctcagccgaagatgacgttaaggccataaaggatctccttgatcagaccaataacttgctgcacaagctgaagatctttcatatcagccagtcggaagacaaagcaaatatccttgacaaggacgagcatttctatctggtgttcgaagaatgctatttcgagctggccaatatcgtgcctctgtacaataagatccgcaattatatcacgcagaagccgtatagcgacgagaagttcaagctgaatttcgagaactccaccttggccaatggttgggataagaacaaagaaccggacaatacggccatcttgtttatcaaagatgacaagtactatctgggagtgatgaataagaagaacaataagatcttcgatgacaaagccattaaggagaataaaggggaaggttacaagaagattgtctataaactgctccccggggccaacaagatgctgcccaaagtttttttcagtgccaagagcatcaagttttataatccctcagaagacatactgagaatcagaaaccactcgacccataccaagaacggctctccgcagaaggggtacgagaaattcgaattcaacatcgaagactgtagaaagttcatcgatttttacaagcaatcgatatcaaagcaccctgaatggaaggattttggttttcgctttagtgacactcagcggtataatagcattgatgagttctaccgcgaagttgagaatcagggatacaaattgacattcgagaatatcagcgaatcgtacattgatagcgtcgtcaaccaggggaaactttacctcttccagatttataataaagacttctcggcgtactccaagggaagaccaaatcttcatactctgtattggaaggcactcttcgatgagagaaatcttcaggatgtcgtttataaacttaatggggaagcggagctgttctaccgcaagcagagcatccctaagaagatcacgcaccccgcgaaggaggcgatagccaataagaacaaggataacccgaagaaggagtccgtcttcgaatatgacctgatcaaggataagagattcactgaggacaaattcttcttccattgccctattacgattaattttaaatcgagcggcgcgaataaattcaacgacgagatcaacctgctcctcaaagagaaggccaatgatgtccacattctctcaatcgacagaggggagaggcaccttgcctactatacgctcgttgatggtaaaggtaacatcattaagcaggacacgttcaacatcatcgggaacgaccgcatgaagacaaactatcacgataaattggcggcaatcgagaaggatcgggatagtgccaggaaggactggaagaagattaataacatcaaggagatgaaggagggatatctttctcaagtcgtccacgagatcgcgaagctggttatcgagtacaacgccatagtggtcttcgaagatctgaacttcggattcaagagagggagatttaaggtggagaagcaagtttatcagaaactggagaagatgctgatagagaagctcaactacctcgtgtttaaagacaacgagtttgacaagacaggtggggtgttgagggcctatcagctcacggcccccttcgagaccttcaagaagatgggaaagcagacggggataatctactacgtccctgctggcttcacttcgaagatctgcccagttacaggattcgttaaccagttgtatcccaaatacgagtccgtctcaaagtcacaagaattcttttctaaattcgataagatctgctacaacctggataagggctacttcgagtttagctttgactataagaatttcggggacaaagcggcaaaggggaagtggacaatcgcaagttttggctcccggctgattaactttcggaattctgacaagaatcacaactgggatactagagaggtctatccaactaaagagttggagaagttgctcaaggactactccattgaatatggtcacggggaatgcattaaagcggccatctgcggagagtccgataagaagttctttgccaaacttacatcggtcttgaacaccatactgcagatgcggaacagcaagacgggaacggaactcgactaccttatctcacctgtggcggatgttaatggaaactttttcgattcgaggcaggcgcccaagaacatgccgcaagatgcagatgccaatggagcatatcacatcggtcttaaagggctcatgctgcttggccgcatcaagaacaaccaagaggggaagaagttgaacctggttattaagaacgaagaatacttcgaatttgtccagaaccgcaacaac

SEQ ID NO: 51 - DNA, 3900 bp, synthetic construct
  misc_feature 1..3900: note = Synthetic polynucleotide.Codon optimized FnCpf1-CO7
  source 1..3900: mol_type = other DNA
atgtccatctatcaggaatttgttaacaagtactctcttagcaaaactcttaggttcgaattgatacctcagggaaagacacttgagaatattaaggcgcgcgggctgatacttgatgatgaaaagcgggcaaaggactataagaaagctaagcaaataattgataagtaccaccagttttttattgaagagatcctgtcctccgtttgtatatccgaagacttgcttcagaattactcagatgtttattttaaattgaagaaatctgacgatgataatcttcaaaaggatttcaaatcggcaaaagacacaatcaaaaaacagattagcgagtacatcaaggactccgaaaagtttaagaatctctttaatcagaatcttatagacgcaaagaaaggacaggaatcggacttgattttgtggttgaagcagtccaaggataacgggatagaactctttaaagccaactccgatataacggacatcgacgaagccctcgaaatcattaagtcgtttaaaggttggaccacgtacttcaaaggattccacgagaacagaaagaacgtttactcgagcaacgacattcctactagcatcatatatagaatagtggatgataatttgcccaaattccttgagaacaaggcaaaatatgaatccctgaaagacaaggcgcccgaagctatcaactacgagcagataaagaaagatctggctgaggagctgacgtttgacattgattacaaaacaagcgaggtcaaccagagggttttttcgctggacgaggtttttgaaatagcgaattttaataactacctgaaccaatccggcatcactaaatttaacacaatcataggggggaagttcgtgaacggagagaacacaaagcgcaaagggattaatgagtacatcaatttgtacagccagcagatcaatgacaaaacgttgaagaaatataagatgagcgtcttgtttaagcagattctctcggatacggaatccaaatcattcgtcattgacaagctggaggatgacagcgatgtggtgacaactatgcagtccttctatgaacaaattgcagcttttaaaacggtcgaagagaagtcgattaaagaaacgctttcgctcctgttcgatgacctgaaagcgcagaagctggacctttcgaagatatatttcaaaaacgataagtcactcacggaccttagccaacaggttttcgatgactattctgtcatagggactgctgtgcttgagtatattactcagcaaatagcccccaaaaacctggacaacccgtctaagaaggaacaagagctgatagcgaagaaaacagagaaagctaaatatctttcacttgaaactataaagcttgcactggaggaattcaacaaacatcgcgacatcgacaagcagtgcagatttgaggagatcttggccaacttcgcagctattccaatgatttttgacgagatagcacagaataaggacaacctggcacagattagcataaaataccagaatcaggggaagaaggatcttcttcaggcttcggctgaggacgatgtcaaagccatcaaagatctgctcgaccagaccaacaatttgttgcataaactcaagatcttccacatatcgcagtccgaagataaagcgaacatacttgacaaagacgaacatttttatcttgttttcgaggagtgttattttgagttggcaaacatcgttcccctctacaacaaaattcgcaactatataactcagaagccctattctgacgagaaattcaaacttaatttcgaaaacagcactctcgcaaacggctgggataaaaacaaggaacccgacaacaccgccatactttttattaaggatgataaatattatttgggcgtgatgaataaaaagaacaataaaatattcgatgataaagcaattaaggaaaataaaggggaagggtataaaaaaatcgtgtataagctgcttccaggagctaataaaatgctgccaaaagtcttcttctctgccaagtcgatcaagttttacaatccttctgaagatattttgcggatcagaaatcactctactcacactaaaaacggttcaccccagaaaggatacgagaagtttgagttcaacatcgaagactgtcggaagtttatcgacttttacaagcagtctatatcaaaacaccccgaatggaaagattttggttttcggttcagcgacacgcagagatataattcaattgatgagttctacagggaggtggagaaccaagggtataaacttacttttgagaacatttccgaatcctatattgattcggtggtcaatcaggggaaactgtacctgtttcagatatataacaaagacttctccgcgtattctaaaggacggcccaatctccatactctttattggaaggcgctgtttgacgagcggaaccttcaggatgttgtctataagttgaacggggaagctgagctgttctatcggaagcagtctattccaaaaaagataacgcaccccgcgaaggaggcaattgcaaacaagaacaaagacaatccaaagaaggagtcggtgtttgagtacgatctgataaaagacaaaaggtttaccgaggacaagttttttttccactgtccgatcaccatcaattttaagtcttccggcgccaacaaattcaatgacgaaataaatctgctgctcaaggaaaaggcaaatgatgttcatattctgtcgatagaccgcggggaaagacacctcgcgtattatacattggtcgatgggaaaggcaatattatcaaacaagacaccttcaatatcattggtaacgataggatgaaaacgaactatcatgataaacttgcagctattgaaaaggacagagactcggctcggaaagattggaaaaagatcaataacatcaaggaaatgaaggaagggtatctctcccaggtcgttcatgaaatcgccaaactggttattgagtacaatgctatagtggtttttgaagatcttaattttgggtttaagagagggagatttaaagtcgagaaacaggtttatcaaaaacttgaaaaaatgttgatagaaaaattgaactatcttgtgtttaaggacaatgagttcgataaaaccgggggggttttgagagcttatcaactgacggctcccttcgagacattcaaaaaaatggggaagcagaccggcatcatttattatgtgcccgcgggcttcacttctaaaatttgtcctgtgacagggttcgtgaaccaattgtaccccaagtacgaaagcgtctccaagtcccaagaattctttagcaagtttgacaaaatttgctataacctggacaaagggtactttgagttttcctttgattataagaatttcggggataaagctgcgaaaggtaagtggacgatagcctctttcgggtcgcgccttattaacttcaggaattccgacaaaaaccataattgggacacccgcgaggtctaccctacaaaggaactcgaaaagttgcttaaggattattcaatagagtatggtcatggcgagtgtattaaggctgctatctgtggtgagtcagataagaaattcttcgcgaaattgacatctgttttgaacaccatcctccaaatgaggaactctaaaacggggacggagcttgattatctcatctcacccgttgctgatgtgaacggtaatttctttgattcacgccaagccccgaagaacatgccccaagacgccgatgctaacggcgcttatcatatagggctgaaggggctcatgcttctggggcgcatcaaaaataaccaagaagggaagaaactcaatctggttatcaaaaatgaggaatatttcgaattcgtgcaaaaccgcaacaat

SEQ ID NO: 52 - DNA, 705 bp, Entacmaea quadricolor
  source 1..705: mol_type = unassigned DNA
gtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgctaagctgaaggtgaccaagggtggccccctgcccttcgcctgggacatcctgtcccctcatttcacctacggctccaaggcctacgtgaagcaccccgccgacatccccgactacttcaagctgtccttccccgagggcttcaagtgggagcgcgtgatgaactacgaggacggcggcgtggtgaccgtgacccaggactcctccctgcaggacggcgagttcatctacaaggtgaagctgcgcggcaccaacttcccctccgacggccccgtgatgcagaagaagaccatgggctgggaggcctcctccgagcggatgtaccccgaggacggtgccctgaagggcaagatcaagatgaggctgaagctgaaggacggcggccactacacctccgaggtcaagaccacctacaaggccaagaagcccgtgcagctgcccggcgcctacatcgtcgacatcaagttggacatcacctcccacaacgaggactacaccatcgtggaacagtacgaacgcgccgagggccgccactccaccggcggcatggacgagctgtacaag

SEQ ID NO: 53 - DNA, 4674 bp, synthetic construct
  misc_feature 1..4674: note = Synthetic polynucleotide.NLS-FnCpf1-CO1-mOR-NLS
  source 1..4674: mol_type = other DNA
ggtagcaaaaagaggcgtatcaagcaggacatgtctatctatcaagagtttgtcaacaagtacagcttaagtaagacacttcgcttcgagttgatccctcaggggaagacactggaaaacatcaaggcgaggggccttatcctggacgacgagaagcgggctaaagactacaaaaaagctaaacagataattgacaagtatcaccaattttttattgaggagatcctctcctctgtgtgcataagtgaggatctgctccagaactattcggatgtttactttaaactcaagaagtccgatgatgacaaccttcagaaggacttcaagtccgccaaagacacgattaagaaacaaatcagtgagtacatcaaggatagcgagaaattcaagaacctgttcaaccagaacttaattgatgccaaaaagggccaagaatccgacctcatcctgtggttaaagcaatctaaagacaacggtattgagctgttcaaggcaaacagcgacattacagacattgacgaggccctagagatcatcaagtcattcaaaggctggacgacttactttaaaggttttcacgagaaccgtaagaatgtttactcaagtaacgatataccaacgagcattatctaccgaatagtggatgataacctaccgaagttccttgagaacaaagcgaagtacgagtctctcaaagacaaggcccctgaggccatcaactacgagcagattaagaaggatctcgccgaggagctaaccttcgacattgactacaaaacatcggaagtgaatcagagggtgttctcgcttgatgaagtattcgagattgctaacttcaacaattacctgaaccagagtggtattactaagttcaacacaatcattggaggcaaattcgtgaacggcgaaaacacaaagcgaaaagggataaacgagtacattaacttgtacagccagcagatcaacgataagacactcaagaagtataagatgtctgtgctgttcaaacaaatcttaagcgacacggaaagcaagtcgttcgtaattgacaagctggaagacgattctgacgtggttacaaccatgcagtccttttacgagcagattgccgcattcaagaccgtggaggaaaagtcgatcaaggaaacactttcgttgcttttcgacgaccttaaagctcagaagctcgacttaagcaagatatactttaagaacgataagagcttgacagacttgagccagcaagtctttgacgactacagcgttatcggaactgccgttctggagtacataacacagcagatcgcacccaagaaccttgacaacccttccaagaaagaacaagagttgatcgccaagaagactgaaaaggctaagtacctctctctggagactatcaagctcgctcttgaggagtttaacaagcacagggacattgacaagcaatgccgattcgaggaaatactggcaaacttcgcagccatacccatgatattcgatgagatagcccagaacaaggataacttggcccaaatctcgattaagtatcagaaccagggcaaaaaggaccttctacaggctagtgcagaggacgatgtgaaggctattaaggacttattagatcagacaaacaaccttctgcataagctcaagatattccatatctcccagtcagaggacaaggccaacattctggataaggacgagcacttctatctcgtattcgaggaatgttactttgagctggccaatatcgttcccttgtacaacaaaatccggaactacatcacacagaagccctacagtgatgagaagttcaaattgaactttgaaaactcaacacttgctaatggttgggacaagaataaggaacctgacaacactgccatcctctttattaaagatgataagtactacctcggggtgatgaataagaagaacaacaaaatcttcgatgacaaagctattaaggagaacaaaggtgaagggtacaagaagattgtctacaaactgctgcctggtgccaacaaaatgctaccaaaggtatttttcagcgccaaatctattaagttctacaatccaagcgaggatattctccggatacggaatcactctacacataccaagaatggaagtccacaaaagggttacgagaaattcgagttcaacattgaagactgccggaaattcattgacttctacaagcaatccatctctaaacatcctgaatggaaagacttcggtttccgcttcagtgatactcaacggtacaattcaattgacgaattctaccgtgaggttgagaaccaggggtacaaactgaccttcgagaacatatcagagagctacatcgactcagtggttaatcaggggaagctatatctgtttcaaatctacaacaaagactttagtgcctactctaaagggcggccaaacttacacacactttactggaaggcactattcgacgaacgcaatctacaagatgtagtttacaaattgaacggtgaggctgagttgttctaccgtaaacaatctatacccaagaagataacacaccctgctaaagaggcaattgcaaacaaaaacaaggataatcccaaaaaggagtctgtctttgagtatgacctcattaaggataagcggttcacggaggacaagttcttcttccattgtccaataaccatcaacttcaaatcatccggcgcaaacaaattcaatgacgagatcaacctgttactaaaggagaaggctaacgatgttcacatcttatctattgatcgaggtgagagacacctagcctactacactttagtggatgggaaggggaacatcatcaagcaagacaccttcaacatcattgggaacgacaggatgaagactaactaccatgataagctcgccgctattgaaaaggacagggactctgccaggaaggactggaaaaaaattaacaatattaaagagatgaaggagggctacctgagccaagtagtccatgagatagcaaaactggtgattgagtacaacgcaatagtcgtattcgaggacttaaacttcggcttcaaacgtgggcggtttaaggtggagaaacaagtctatcagaaattggagaagatgctaatcgagaagctcaactacctcgtgtttaaagacaacgagtttgacaaaactggaggagtcctgcgggcataccaactgaccgcacccttcgagacattcaagaagatgggaaagcagactggcatcatctattacgtgccagcgggttttacttccaaaatctgtccagttacaggcttcgtgaaccagttgtacccgaagtacgagtctgtttccaagtcacaggaattcttctcaaagtttgacaagatatgttacaatctcgataagggatactttgagtttagtttcgactacaagaactttggcgataaggccgcaaaagggaaatggacaattgcatccttcgggtcacgccttattaactttcgtaactcagacaagaaccacaattgggacaccagggaggtgtaccctactaaggagctggagaagctacttaaagactactcgattgagtacggacatggagagtgcatcaaggcagcaatatgtggggaatctgacaaaaagttctttgccaagctgacctctgtactgaacactattctccaaatgagaaatagtaagactggcacagagttggactacctgatctctccagtggctgacgttaatgggaattttttcgactcaagacaagctcccaagaatatgccacaggacgcagatgcaaacggggcatatcacatcgggcttaaaggactcatgctactagggcggatcaagaataatcaggagggcaaaaagctgaacctagtcatcaagaacgaggagtacttcgaatttgtccagaatcgtaacaacggatctggagtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgctaagctgaaggtgaccaagggtggccccctgcccttcgcctgggacatcctgtcccctcatttcacctacggctccaaggcctacgtgaagcaccccgccgacatccccgactacttcaagctgtccttccccgagggcttcaagtgggagcgcgtgatgaactacgaggacggcggcgtggtgaccgtgacccaggactcctccctgcaggacggcgagttcatctacaaggtgaagctgcgcggcaccaacttcccctccgacggccccgtgatgcagaagaagaccatgggctgggaggcctcctccgagcggatgtaccccgaggacggtgccctgaagggcaagatcaagatgaggctgaagctgaaggacggcggccactacacctccgaggtcaagaccacctacaaggccaagaagcccgtgcagctgcccggcgcctacatcgtcgacatcaagttggacatcacctcccacaacgaggactacaccatcgtggaacagtacgaacgcgccgagggccgccactccaccggcggcatggacgagctgtacaagggatctaagaagcgtaggatcaagcaagat

SEQ ID NO: 54 - DNA, 33 bp, synthetic construct
  misc_feature 1..33: note = Synthetic polynucleotide.MGSS7H His tag comprising sevenHistidine residues
  source 1..33: mol_type = other DNA
atgggcagcagccatcatcaccaccatcaccat

SEQ ID NO: 55 - DNA, 4710 bp, synthetic construct
  misc_feature 1..4710: note = Synthetic polynucleotide.MGSS7H-NLS-FnCpf1-CO1-mOR-NLS
  source 1..4710: mol_type = other DNA
atgggcagcagccatcatcaccaccatcaccatatgggtagcaaaaagaggcgtatcaagcaggacatgtctatctatcaagagtttgtcaacaagtacagcttaagtaagacacttcgcttcgagttgatccctcaggggaagacactggaaaacatcaaggcgaggggccttatcctggacgacgagaagcgggctaaagactacaaaaaagctaaacagataattgacaagtatcaccaattttttattgaggagatcctctcctctgtgtgcataagtgaggatctgctccagaactattcggatgtttactttaaactcaagaagtccgatgatgacaaccttcagaaggacttcaagtccgccaaagacacgattaagaaacaaatcagtgagtacatcaaggatagcgagaaattcaagaacctgttcaaccagaacttaattgatgccaaaaagggccaagaatccgacctcatcctgtggttaaagcaatctaaagacaacggtattgagctgttcaaggcaaacagcgacattacagacattgacgaggccctagagatcatcaagtcattcaaaggctggacgacttactttaaaggttttcacgagaaccgtaagaatgtttactcaagtaacgatataccaacgagcattatctaccgaatagtggatgataacctaccgaagttccttgagaacaaagcgaagtacgagtctctcaaagacaaggcccctgaggccatcaactacgagcagattaagaaggatctcgccgaggagctaaccttcgacattgactacaaaacatcggaagtgaatcagagggtgttctcgcttgatgaagtattcgagattgctaacttcaacaattacctgaaccagagtggtattactaagttcaacacaatcattggaggcaaattcgtgaacggcgaaaacacaaagcgaaaagggataaacgagtacattaacttgtacagccagcagatcaacgataagacactcaagaagtataagatgtctgtgctgttcaaacaaatcttaagcgacacggaaagcaagtcgttcgtaattgacaagctggaagacgattctgacgtggttacaaccatgcagtccttttacgagcagattgccgcattcaagaccgtggaggaaaagtcgatcaaggaaacactttcgttgcttttcgacgaccttaaagctcagaagctcgacttaagcaagatatactttaagaacgataagagcttgacagacttgagccagcaagtctttgacgactacagcgttatcggaactgccgttctggagtacataacacagcagatcgcacccaagaaccttgacaacccttccaagaaagaacaagagttgatcgccaagaagactgaaaaggctaagtacctctctctggagactatcaagctcgctcttgaggagtttaacaagcacagggacattgacaagcaatgccgattcgaggaaatactggcaaacttcgcagccatacccatgatattcgatgagatagcccagaacaaggataacttggcccaaatctcgattaagtatcagaaccagggcaaaaaggaccttctacaggctagtgcagaggacgatgtgaaggctattaaggacttattagatcagacaaacaaccttctgcataagctcaagatattccatatctcccagtcagaggacaaggccaacattctggataaggacgagcacttctatctcgtattcgaggaatgttactttgagctggccaatatcgttcccttgtacaacaaaatccggaactacatcacacagaagccctacagtgatgagaagttcaaattgaactttgaaaactcaacacttgctaatggttgggacaagaataaggaacctgacaacactgccatcctctttattaaagatgataagtactacctcggggtgatgaataagaagaacaacaaaatcttcgatgacaaagctattaaggagaacaaaggtgaagggtacaagaagattgtctacaaactgctgcctggtgccaacaaaatgctaccaaaggtatttttcagcgccaaatctattaagttctacaatccaagcgaggatattctccggatacggaatcactctacacataccaagaatggaagtccacaaaagggttacgagaaattcgagttcaacattgaagactgccggaaattcattgacttctacaagcaatccatctctaaacatcctgaatggaaagacttcggtttccgcttcagtgatactcaacggtacaattcaattgacgaattctaccgtgaggttgagaaccaggggtacaaactgaccttcgagaacatatcagagagctacatcgactcagtggttaatcaggggaagctatatctgtttcaaatctacaacaaagactttagtgcctactctaaagggcggccaaacttacacacactttactggaaggcactattcgacgaacgcaatctacaagatgtagtttacaaattgaacggtgaggctgagttgttctaccgtaaacaatctatacccaagaagataacacaccctgctaaagaggcaattgcaaacaaaaacaaggataatcccaaaaaggagtctgtctttgagtatgacctcattaaggataagcggttcacggaggacaagttcttcttccattgtccaataaccatcaacttcaaatcatccggcgcaaacaaattcaatgacgagatcaacctgttactaaaggagaaggctaacgatgttcacatcttatctattgatcgaggtgagagacacctagcctactacactttagtggatgggaaggggaacatcatcaagcaagacaccttcaacatcattgggaacgacaggatgaagactaactaccatgataagctcgccgctattgaaaaggacagggactctgccaggaaggactggaaaaaaattaacaatattaaagagatgaaggagggctacctgagccaagtagtccatgagatagcaaaactggtgattgagtacaacgcaatagtcgtattcgaggacttaaacttcggcttcaaacgtgggcggtttaaggtggagaaacaagtctatcagaaattggagaagatgctaatcgagaagctcaactacctcgtgtttaaagacaacgagtttgacaaaactggaggagtcctgcgggcataccaactgaccgcacccttcgagacattcaagaagatgggaaagcagactggcatcatctattacgtgccagcgggttttacttccaaaatctgtccagttacaggcttcgtgaaccagttgtacccgaagtacgagtctgtttccaagtcacaggaattcttctcaaagtttgacaagatatgttacaatctcgataagggatactttgagtttagtttcgactacaagaactttggcgataaggccgcaaaagggaaatggacaattgcatccttcgggtcacgccttattaactttcgtaactcagacaagaaccacaattgggacaccagggaggtgtaccctactaaggagctggagaagctacttaaagactactcgattgagtacggacatggagagtgcatcaaggcagcaatatgtggggaatctgacaaaaagttctttgccaagctgacctctgtactgaacactattctccaaatgagaaatagtaagactggcacagagttggactacctgatctctccagtggctgacgttaatgggaattttttcgactcaagacaagctcccaagaatatgccacaggacgcagatgcaaacggggcatatcacatcgggcttaaaggactcatgctactagggcggatcaagaataatcaggagggcaaaaagctgaacctagtcatcaagaacgaggagtacttcgaatttgtccagaatcgtaacaacggatctggagtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgctaagctgaaggtgaccaagggtggccccctgcccttcgcctgggacatcctgtcccctcatttcacctacggctccaaggcctacgtgaagcaccccgccgacatccccgactacttcaagctgtccttccccgagggcttcaagtgggagcgcgtgatgaactacgaggacggcggcgtggtgaccgtgacccaggactcctccctgcaggacggcgagttcatctacaaggtgaagctgcgcggcaccaacttcccctccgacggccccgtgatgcagaagaagaccatgggctgggaggcctcctccgagcggatgtaccccgaggacggtgccctgaagggcaagatcaagatgaggctgaagctgaaggacggcggccactacacctccgaggtcaagaccacctacaaggccaagaagcccgtgcagctgcccggcgcctacatcgtcgacatcaagttggacatcacctcccacaacgaggactacaccatcgtggaacagtacgaacgcgccgagggccgccactccaccggcggcatggacgagctgtacaagggatctaagaagcgtaggatcaagcaagat

SEQ ID NO: 56 - DNA, 6465 bp, synthetic construct
  misc_feature 1..6465: note = Synthetic polynucleotide.Expression cassette comprising 35Spromoter cassette, MGSS7H-NLS-FnCpf1-CO1-mOr-NLS and NOStranscription termination sequence
  source 1..6465: mol_type = other DNA
ggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacaagctgactctagcagatctaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcctctagaggatccaggagcaaccatgggcagcagccatcatcaccaccatcaccatatgggtagcaaaaagaggcgtatcaagcaggacatgtctatctatcaagagtttgtcaacaagtacagcttaagtaagacacttcgcttcgagttgatccctcaggggaagacactggaaaacatcaaggcgaggggccttatcctggacgacgagaagcgggctaaagactacaaaaaagctaaacagataattgacaagtatcaccaattttttattgaggagatcctctcctctgtgtgcataagtgaggatctgctccagaactattcggatgtttactttaaactcaagaagtccgatgatgacaaccttcagaaggacttcaagtccgccaaagacacgattaagaaacaaatcagtgagtacatcaaggatagcgagaaattcaagaacctgttcaaccagaacttaattgatgccaaaaagggccaagaatccgacctcatcctgtggttaaagcaatctaaagacaacggtattgagctgttcaaggcaaacagcgacattacagacattgacgaggccctagagatcatcaagtcattcaaaggctggacgacttactttaaaggttttcacgagaaccgtaagaatgtttactcaagtaacgatataccaacgagcattatctaccgaatagtggatgataacctaccgaagttccttgagaacaaagcgaagtacgagtctctcaaagacaaggcccctgaggccatcaactacgagcagattaagaaggatctcgccgaggagctaaccttcgacattgactacaaaacatcggaagtgaatcagagggtgttctcgcttgatgaagtattcgagattgctaacttcaacaattacctgaaccagagtggtattactaagttcaacacaatcattggaggcaaattcgtgaacggcgaaaacacaaagcgaaaagggataaacgagtacattaacttgtacagccagcagatcaacgataagacactcaagaagtataagatgtctgtgctgttcaaacaaatcttaagcgacacggaaagcaagtcgttcgtaattgacaagctggaagacgattctgacgtggttacaaccatgcagtccttttacgagcagattgccgcattcaagaccgtggaggaaaagtcgatcaaggaaacactttcgttgcttttcgacgaccttaaagctcagaagctcgacttaagcaagatatactttaagaacgataagagcttgacagacttgagccagcaagtctttgacgactacagcgttatcggaactgccgttctggagtacataacacagcagatcgcacccaagaaccttgacaacccttccaagaaagaacaagagttgatcgccaagaagactgaaaaggctaagtacctctctctggagactatcaagctcgctcttgaggagtttaacaagcacagggacattgacaagcaatgccgattcgaggaaatactggcaaacttcgcagccatacccatgatattcgatgagatagcccagaacaaggataacttggcccaaatctcgattaagtatcagaaccagggcaaaaaggaccttctacaggctagtgcagaggacgatgtgaaggctattaaggacttattagatcagacaaacaaccttctgcataagctcaagatattccatatctcccagtcagaggacaaggccaacattctggataaggacgagcacttctatctcgtattcgaggaatgttactttgagctggccaatatcgttcccttgtacaacaaaatccggaactacatcacacagaagccctacagtgatgagaagttcaaattgaactttgaaaactcaacacttgctaatggttgggacaagaataaggaacctgacaacactgccatcctctttattaaagatgataagtactacctcggggtgatgaataagaagaacaacaaaatcttcgatgacaaagctattaaggagaacaaaggtgaagggtacaagaagattgtctacaaactgctgcctggtgccaacaaaatgctaccaaaggtatttttcagcgccaaatctattaagttctacaatccaagcgaggatattctccggatacggaatcactctacacataccaagaatggaagtccacaaaagggttacgagaaattcgagttcaacattgaagactgccggaaattcattgacttctacaagcaatccatctctaaacatcctgaatggaaagacttcggtttccgcttcagtgatactcaacggtacaattcaattgacgaattctaccgtgaggttgagaaccaggggtacaaactgaccttcgagaacatatcagagagctacatcgactcagtggttaatcaggggaagctatatctgtttcaaatctacaacaaagactttagtgcctactctaaagggcggccaaacttacacacactttactggaaggcactattcgacgaacgcaatctacaagatgtagtttacaaattgaacggtgaggctgagttgttctaccgtaaacaatctatacccaagaagataacacaccctgctaaagaggcaattgcaaacaaaaacaaggataatcccaaaaaggagtctgtctttgagtatgacctcattaaggataagcggttcacggaggacaagttcttcttccattgtccaataaccatcaacttcaaatcatccggcgcaaacaaattcaatgacgagatcaacctgttactaaaggagaaggctaacgatgttcacatcttatctattgatcgaggtgagagacacctagcctactacactttagtggatgggaaggggaacatcatcaagcaagacaccttcaacatcattgggaacgacaggatgaagactaactaccatgataagctcgccgctattgaaaaggacagggactctgccaggaaggactggaaaaaaattaacaatattaaagagatgaaggagggctacctgagccaagtagtccatgagatagcaaaactggtgattgagtacaacgcaatagtcgtattcgaggacttaaacttcggcttcaaacgtgggcggtttaaggtggagaaacaagtctatcagaaattggagaagatgctaatcgagaagctcaactacctcgtgtttaaagacaacgagtttgacaaaactggaggagtcctgcgggcataccaactgaccgcacccttcgagacattcaagaagatgggaaagcagactggcatcatctattacgtgccagcgggttttacttccaaaatctgtccagttacaggcttcgtgaaccagttgtacccgaagtacgagtctgtttccaagtcacaggaattcttctcaaagtttgacaagatatgttacaatctcgataagggatactttgagtttagtttcgactacaagaactttggcgataaggccgcaaaagggaaatggacaattgcatccttcgggtcacgccttattaactttcgtaactcagacaagaaccacaattgggacaccagggaggtgtaccctactaaggagctggagaagctacttaaagactactcgattgagtacggacatggagagtgcatcaaggcagcaatatgtggggaatctgacaaaaagttctttgccaagctgacctctgtactgaacactattctccaaatgagaaatagtaagactggcacagagttggactacctgatctctccagtggctgacgttaatgggaattttttcgactcaagacaagctcccaagaatatgccacaggacgcagatgcaaacggggcatatcacatcgggcttaaaggactcatgctactagggcggatcaagaataatcaggagggcaaaaagctgaacctagtcatcaagaacgaggagtacttcgaatttgtccagaatcgtaacaacggatctggagtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgctaagctgaaggtgaccaagggtggccccctgcccttcgcctgggacatcctgtcccctcatttcacctacggctccaaggcctacgtgaagcaccccgccgacatccccgactacttcaagctgtccttccccgagggcttcaagtgggagcgcgtgatgaactacgaggacggcggcgtggtgaccgtgacccaggactcctccctgcaggacggcgagttcatctacaaggtgaagctgcgcggcaccaacttcccctccgacggccccgtgatgcagaagaagaccatgggctgggaggcctcctccgagcggatgtaccccgaggacggtgccctgaagggcaagatcaagatgaggctgaagctgaaggacggcggccactacacctccgaggtcaagaccacctacaaggccaagaagcccgtgcagctgcccggcgcctacatcgtcgacatcaagttggacatcacctcccacaacgaggactacaccatcgtggaacagtacgaacgcgccgagggccgccactccaccggcggcatggacgagctgtacaagggatctaagaagcgtaggatcaagcaagattagaacccagcggtactcgctgaggaattcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 57 - DNA, 4674 bp, synthetic construct
  misc_feature 1..4674: note = Synthetic polynucleotide.NLS-FnCpf1-CO2-mOR-NLS
  source 1..4674: mol_type = other DNA
ggtagcaaaaagaggcgtatcaagcaggacatgtccatataccaggagttcgtcaacaagtactcgctcagcaagacgctccgcttcgagctgatcccccagggtaagaccctggagaacatcaaggcgcgcggactcatcctcgacgacgagaagcgggctaaggactacaaaaaggccaagcagatcatcgacaagtaccaccagttcttcatcgaggagatcctctcctccgtctgcatctccgaggacctcctccagaactacagcgacgtctacttcaagctcaaaaagtcggacgacgacaacctccagaaggacttcaagtcggcgaaggacaccatcaagaagcagatcagcgagtacatcaaggactccgagaagttcaagaacctgttcaaccagaacctgatcgacgccaagaagggccaggagtcggacctgatcctgtggctcaagcagagcaaggacaacggcatcgagctcttcaaggccaacagcgacatcacggacatcgacgaggccctggagatcatcaagtcgttcaagggctggacgacctacttcaagggcttccacgagaaccgcaagaacgtctatagctccaacgacatccccacctcgatcatctaccggatcgtggacgacaacctccccaagttcctggagaacaaggcgaagtacgagtcgttgaaggacaaggcgccggaggcgatcaactacgagcagatcaagaaggacctggccgaggagctgaccttcgacatcgactacaagacgtcggaggtgaaccagcgggtgttcagtctggacgaggtcttcgagatcgcgaacttcaacaactacctgaaccagtcggggatcaccaagttcaacacgatcataggcggcaagttcgtgaacggcgagaacaccaagcgcaaggggataaacgagtacatcaacctgtacagccagcagatcaacgacaagacgctcaagaagtacaagatgagcgtgctcttcaagcagatcctcagtgacaccgagtccaagagcttcgtgatcgacaagctggaggacgacagcgacgtcgtgacaaccatgcagagcttctacgagcagatcgccgcgttcaagaccgtcgaggagaagtcaatcaaggagacgctctccttgctgttcgacgacctgaaggcacaaaagctggacctcagcaagatctacttcaagaacgacaagtccctgaccgacctgagccagcaggttttcgacgactactccgtcatcgggactgccgtcctggagtacatcacccagcagatcgctccgaagaacctcgacaacccgtccaagaaggagcaggagctgattgcgaagaagacagagaaggccaagtacctctccctcgagaccatcaagctcgccctggaggagttcaacaagcacagggacattgacaagcagtgccgtttcgaggagatcctggccaacttcgcggccatccccatgatcttcgacgagatcgcccagaacaaggacaacctggcgcagatctctatcaagtaccagaaccaggggaagaaggacttgctccaggcctcagcggaggacgacgtgaaggccatcaaggacctgctcgaccagacgaacaacttgctccacaagctcaagatctttcacatcagccagagcgaggacaaggccaacatcctcgacaaggacgagcacttctacctcgtgttcgaggagtgctacttcgagctggccaacatcgtgccactttacaacaagatccgcaactacatcacgcagaagccgtactccgacgagaagttcaagctgaacttcgagaactccaccctcgccaacggctgggacaagaacaaggagccggacaacaccgcgatcctgttcataaaggacgacaagtactacttgggggtcatgaacaagaagaacaacaagatattcgacgacaaggccatcaaggagaacaagggggagggctacaagaagatcgtctacaagctcctccccggcgcgaacaagatgctgcctaaggtcttcttttccgccaagagtatcaagttctacaacccctccgaggacatcctccgcatccggaaccacagcacgcacacaaagaacggctcgcctcagaagggctacgagaagttcgagttcaacatcgaggactgccggaagttcatcgacttctacaagcagagcatctccaagcacccggagtggaaggactttggcttcaggttctcagacacccagcggtacaactccatcgacgagttctaccgcgaggtggagaaccagggctacaagctgaccttcgagaatatatcagagtcgtacatcgacagcgtggtgaaccagggcaagttgtacctgttccagatctacaacaaggacttctccgcctactcaaaggggcgtccaaacctgcacacgctgtactggaaggcgctcttcgacgagcgcaacctacaagatgttgtatacaagctcaacggcgaggcggaactgttctataggaagcagtcgatccccaagaagattacgcacccggctaaggaggccatcgccaacaagaacaaggacaaccccaagaaggagtccgtgttcgagtacgacctcatcaaggacaagaggttcacggaggacaagtttttcttccactgcccaatcactatcaatttcaagtcgagcggagccaacaagttcaacgacgagataaacctgctcctcaaggagaaggccaatgacgtgcacatcctctccatcgaccggggcgagcggcacctggcgtactacacgctggtggacggcaagggcaacatcatcaagcaggacaccttcaacatcatcgggaacgaccgcatgaagaccaactaccacgacaagctcgccgccatcgagaaggacagggactccgcgcgcaaggactggaagaagattaacaacatcaaggagatgaaggagggctacctcagccaggtggtccacgagatcgccaagctcgtcattgagtacaacgccatcgtcgtcttcgaggacctgaatttcggcttcaagcgcggccggttcaaggtggagaagcaggtctaccagaagcttgagaagatgctgatcgagaagctgaactacctggtgttcaaggacaacgagttcgacaagaccggcggagtgctgcgcgcctaccagctcacggcgcctttcgagacgttcaagaagatgggcaagcagacgggcatcatctactacgtgcccgccggcttcacctctaagatctgcccagtgaccggcttcgttaaccagctgtacccgaagtacgagagcgtgtccaagtcccaggagttcttctccaagttcgacaagatttgttacaacctcgacaagggctacttcgagttttcgttcgactacaagaactttggcgacaaggcggccaaggggaagtggaccatcgcctctttcggcagcaggctcatcaatttccggaactccgacaagaaccacaactgggacacgcgcgaggtgtacccgacgaaggagctggagaagctgctcaaggactactccatcgagtacggccacggcgagtgcatcaaggcggcgatctgcggggagagcgacaagaagttctttgccaagctgaccagcgtgctgaacaccatcctccagatgcggaactccaagaccggcaccgagctggactacctgatctccccggtcgcggacgtcaacgggaacttcttcgactcccgacaggctcccaagaacatgccccaggacgccgacgcgaacggcgcgtaccacatcggcctcaagggcctgatgctgctggggcgcatcaagaacaaccaggagggcaagaagctgaacctcgtgatcaagaacgaggaatacttcgagttcgtgcagaaccgcaacaacggatctggagtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgctaagctgaaggtgaccaagggtggccccctgcccttcgcctgggacatcctgtcccctcatttcacctacggctccaaggcctacgtgaagcaccccgccgacatccccgactacttcaagctgtccttccccgagggcttcaagtgggagcgcgtgatgaactacgaggacggcggcgtggtgaccgtgacccaggactcctccctgcaggacggcgagttcatctacaaggtgaagctgcgcggcaccaacttcccctccgacggccccgtgatgcagaagaagaccatgggctgggaggcctcctccgagcggatgtaccccgaggacggtgccctgaagggcaagatcaagatgaggctgaagctgaaggacggcggccactacacctccgaggtcaagaccacctacaaggccaagaagcccgtgcagctgcccggcgcctacatcgtcgacatcaagttggacatcacctcccacaacgaggactacaccatcgtggaacagtacgaacgcgccgagggccgccactccaccggcggcatggacgagctgtacaagggatctaagaagcgtaggatcaagcaagat

SEQ ID NO: 58 - DNA, 4710 bp, synthetic construct
  misc_feature 1..4710: note = Synthetic polynucleotide. MGSS7H-NLS-FnCpf1-CO2-mOR-NLS
  source 1..4710: mol_type = other DNA
atgggcagcagccatcatcaccaccatcaccatatgggtagcaaaaagaggcgtatcaagcaggacatgtccatataccaggagttcgtcaacaagtactcgctcagcaagacgctccgcttcgagctgatcccccagggtaagaccctggagaacatcaaggcgcgcggactcatcctcgacgacgagaagcgggctaaggactacaaaaaggccaagcagatcatcgacaagtaccaccagttcttcatcgaggagatcctctcctccgtctgcatctccgaggacctcctccagaactacagcgacgtctacttcaagctcaaaaagtcggacgacgacaacctccagaaggacttcaagtcggcgaaggacaccatcaagaagcagatcagcgagtacatcaaggactccgagaagttcaagaacctgttcaaccagaacctgatcgacgccaagaagggccaggagtcggacctgatcctgtggctcaagcagagcaaggacaacggcatcgagctcttcaaggccaacagcgacatcacggacatcgacgaggccctggagatcatcaagtcgttcaagggctggacgacctacttcaagggcttccacgagaaccgcaagaacgtctatagctccaacgacatccccacctcgatcatctaccggatcgtggacgacaacctccccaagttcctggagaacaaggcgaagtacgagtcgttgaaggacaaggcgccggaggcgatcaactacgagcagatcaagaaggacctggccgaggagctgaccttcgacatcgactacaagacgtcggaggtgaaccagcgggtgttcagtctggacgaggtcttcgagatcgcgaacttcaacaactacctgaaccagtcggggatcaccaagttcaacacgatcataggcggcaagttcgtgaacggcgagaacaccaagcgcaaggggataaacgagtacatcaacctgtacagccagcagatcaacgacaagacgctcaagaagtacaagatgagcgtgctcttcaagcagatcctcagtgacaccgagtccaagagcttcgtgatcgacaagctggaggacgacagcgacgtcgtgacaaccatgcagagcttctacgagcagatcgccgcgttcaagaccgtcgaggagaagtcaatcaaggagacgctctccttgctgttcgacgacctgaaggcacaaaagctggacctcagcaagatctacttcaagaacgacaagtccctgaccgacctgagccagcaggttttcgacgactactccgtcatcgggactgccgtcctggagtacatcacccagcagatcgctccgaagaacctcgacaacccgtccaagaaggagcaggagctgattgcgaagaagacagagaaggccaagtacctctccctcgagaccatcaagctcgccctggaggagttcaacaagcacagggacattgacaagcagtgccgtttcgaggagatcctggccaacttcgcggccatccccatgatcttcgacgagatcgcccagaacaaggacaacctggcgcagatctctatcaagtaccagaaccaggggaagaaggacttgctccaggcctcagcggaggacgacgtgaaggccatcaaggacctgctcgaccagacgaacaacttgctccacaagctcaagatctttcacatcagccagagcgaggacaaggccaacatcctcgacaaggacgagcacttctacctcgtgttcgaggagtgctacttcgagctggccaacatcgtgccactttacaacaagatccgcaactacatcacgcagaagccgtactccgacgagaagttcaagctgaacttcgagaactccaccctcgccaacggctgggacaagaacaaggagccggacaacaccgcgatcctgttcataaaggacgacaagtactacttgggggtcatgaacaagaagaacaacaagatattcgacgacaaggccatcaaggagaacaagggggagggctacaagaagatcgtctacaagctcctccccggcgcgaacaagatgctgcctaaggtcttcttttccgccaagagtatcaagttctacaacccctccgaggacatcctccgcatccggaaccacagcacgcacacaaagaacggctcgcctcagaagggctacgagaagttcgagttcaacatcgaggactgccggaagttcatcgacttctacaagcagagcatctccaagcacccggagtggaaggactttggcttcaggttctcagacacccagcggtacaactccatcgacgagttctaccgcgaggtggagaaccagggctacaagctgaccttcgagaatatatcagagtcgtacatcgacagcgtggtgaaccagggcaagttgtacctgttccagatctacaacaaggacttctccgcctactcaaaggggcgtccaaacctgcacacgctgtactggaaggcgctcttcgacgagcgcaacctacaagatgttgtatacaagctcaacggcgaggcggaactgttctataggaagcagtcgatccccaagaagattacgcacccggctaaggaggccatcgccaacaagaacaaggacaaccccaagaaggagtccgtgttcgagtacgacctcatcaaggacaagaggttcacggaggacaagtttttcttccactgcccaatcactatcaatttcaagtcgagcggagccaacaagttcaacgacgagataaacctgctcctcaaggagaaggccaatgacgtgcacatcctctccatcgaccggggcgagcggcacctggcgtactacacgctggtggacggcaagggcaacatcatcaagcaggacaccttcaacatcatcgggaacgaccgcatgaagaccaactaccacgacaagctcgccgccatcgagaaggacagggactccgcgcgcaaggactggaagaagattaacaacatcaaggagatgaaggagggctacctcagccaggtggtccacgagatcgccaagctcgtcattgagtacaacgccatcgtcgtcttcgaggacctgaatttcggcttcaagcgcggccggttcaaggtggagaagcaggtctaccagaagcttgagaagatgctgatcgagaagctgaactacctggtgttcaaggacaacgagttcgacaagaccggcggagtgctgcgcgcctaccagctcacggcgcctttcgagacgttcaagaagatgggcaagcagacgggcatcatctactacgtgcccgccggcttcacctctaagatctgcccagtgaccggcttcgttaaccagctgtacccgaagtacgagagcgtgtccaagtcccaggagttcttctccaagttcgacaagatttgttacaacctcgacaagggctacttcgagttttcgttcgactacaagaactttggcgacaaggcggccaaggggaagtggaccatcgcctctttcggcagcaggctcatcaatttccggaactccgacaagaaccacaactgggacacgcgcgaggtgtacccgacgaaggagctggagaagctgctcaaggactactccatcgagtacggccacggcgagtgcatcaaggcggcgatctgcggggagagcgacaagaagttctttgccaagctgaccagcgtgctgaacaccatcctccagatgcggaactccaagaccggcaccgagctggactacctgatctccccggtcgcggacgtcaacgggaacttcttcgactcccgacaggctcccaagaacatgccccaggacgccgacgcgaacggcgcgtaccacatcggcctcaagggcctgatgctgctggggcgcatcaagaacaaccaggagggcaagaagctgaacctcgtgatcaagaacgaggaatacttcgagttcgtgcagaaccgcaacaacggatctggagtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgctaagctgaaggtgaccaagggtggccccctgcccttcgcctgggacatcctgtcccctcatttcacctacggctccaaggcctacgtgaagcaccccgccgacatccccgactacttcaagctgtccttccccgagggcttcaagtgggagcgcgtgatgaactacgaggacggcggcgtggtgaccgtgacccaggactcctccctgcaggacggcgagttcatctacaaggtgaagctgcgcggcaccaacttcccctccgacggccccgtgatgcagaagaagaccatgggctgggaggcctcctccgagcggatgtaccccgaggacggtgccctgaagggcaagatcaagatgaggctgaagctgaaggacggcggccactacacctccgaggtcaagaccacctacaaggccaagaagcccgtgcagctgcccggcgcctacatcgtcgacatcaagttggacatcacctcccacaacgaggactacaccatcgtggaacagtacgaacgcgccgagggccgccactccaccggcggcatggacgagctgtacaagggatctaagaagcgtaggatcaagcaagat

SEQ ID NO: 59 - DNA, 6465 bp, synthetic construct
  misc_feature 1..6465: note = Synthetic polynucleotide.Expression cassette comprising 35Spromoter cassette, MGSS7H-NLS-FnCpf1-CO2-mOr-NLS and NOStranscription termination sequence
  source 1..6465: mol_type = other DNA
ggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacaagctgactctagcagatctaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcctctagaggatccaggagcaaccatgggcagcagccatcatcaccaccatcaccatatgggtagcaaaaagaggcgtatcaagcaggacatgtccatataccaggagttcgtcaacaagtactcgctcagcaagacgctccgcttcgagctgatcccccagggtaagaccctggagaacatcaaggcgcgcggactcatcctcgacgacgagaagcgggctaaggactacaaaaaggccaagcagatcatcgacaagtaccaccagttcttcatcgaggagatcctctcctccgtctgcatctccgaggacctcctccagaactacagcgacgtctacttcaagctcaaaaagtcggacgacgacaacctccagaaggacttcaagtcggcgaaggacaccatcaagaagcagatcagcgagtacatcaaggactccgagaagttcaagaacctgttcaaccagaacctgatcgacgccaagaagggccaggagtcggacctgatcctgtggctcaagcagagcaaggacaacggcatcgagctcttcaaggccaacagcgacatcacggacatcgacgaggccctggagatcatcaagtcgttcaagggctggacgacctacttcaagggcttccacgagaaccgcaagaacgtctatagctccaacgacatccccacctcgatcatctaccggatcgtggacgacaacctccccaagttcctggagaacaaggcgaagtacgagtcgttgaaggacaaggcgccggaggcgatcaactacgagcagatcaagaaggacctggccgaggagctgaccttcgacatcgactacaagacgtcggaggtgaaccagcgggtgttcagtctggacgaggtcttcgagatcgcgaacttcaacaactacctgaaccagtcggggatcaccaagttcaacacgatcataggcggcaagttcgtgaacggcgagaacaccaagcgcaaggggataaacgagtacatcaacctgtacagccagcagatcaacgacaagacgctcaagaagtacaagatgagcgtgctcttcaagcagatcctcagtgacaccgagtccaagagcttcgtgatcgacaagctggaggacgacagcgacgtcgtgacaaccatgcagagcttctacgagcagatcgccgcgttcaagaccgtcgaggagaagtcaatcaaggagacgctctccttgctgttcgacgacctgaaggcacaaaagctggacctcagcaagatctacttcaagaacgacaagtccctgaccgacctgagccagcaggttttcgacgactactccgtcatcgggactgccgtcctggagtacatcacccagcagatcgctccgaagaacctcgacaacccgtccaagaaggagcaggagctgattgcgaagaagacagagaaggccaagtacctctccctcgagaccatcaagctcgccctggaggagttcaacaagcacagggacattgacaagcagtgccgtttcgaggagatcctggccaacttcgcggccatccccatgatcttcgacgagatcgcccagaacaaggacaacctggcgcagatctctatcaagtaccagaaccaggggaagaaggacttgctccaggcctcagcggaggacgacgtgaaggccatcaaggacctgctcgaccagacgaacaacttgctccacaagctcaagatctttcacatcagccagagcgaggacaaggccaacatcctcgacaaggacgagcacttctacctcgtgttcgaggagtgctacttcgagctggccaacatcgtgccactttacaacaagatccgcaactacatcacgcagaagccgtactccgacgagaagttcaagctgaacttcgagaactccaccctcgccaacggctgggacaagaacaaggagccggacaacaccgcgatcctgttcataaaggacgacaagtactacttgggggtcatgaacaagaagaacaacaagatattcgacgacaaggccatcaaggagaacaagggggagggctacaagaagatcgtctacaagctcctccccggcgcgaacaagatgctgcctaaggtcttcttttccgccaagagtatcaagttctacaacccctccgaggacatcctccgcatccggaaccacagcacgcacacaaagaacggctcgcctcagaagggctacgagaagttcgagttcaacatcgaggactgccggaagttcatcgacttctacaagcagagcatctccaagcacccggagtggaaggactttggcttcaggttctcagacacccagcggtacaactccatcgacgagttctaccgcgaggtggagaaccagggctacaagctgaccttcgagaatatatcagagtcgtacatcgacagcgtggtgaaccagggcaagttgtacctgttccagatctacaacaaggacttctccgcctactcaaaggggcgtccaaacctgcacacgctgtactggaaggcgctcttcgacgagcgcaacctacaagatgttgtatacaagctcaacggcgaggcggaactgttctataggaagcagtcgatccccaagaagattacgcacccggctaaggaggccatcgccaacaagaacaaggacaaccccaagaaggagtccgtgttcgagtacgacctcatcaaggacaagaggttcacggaggacaagtttttcttccactgcccaatcactatcaatttcaagtcgagcggagccaacaagttcaacgacgagataaacctgctcctcaaggagaaggccaatgacgtgcacatcctctccatcgaccggggcgagcggcacctggcgtactacacgctggtggacggcaagggcaacatcatcaagcaggacaccttcaacatcatcgggaacgaccgcatgaagaccaactaccacgacaagctcgccgccatcgagaaggacagggactccgcgcgcaaggactggaagaagattaacaacatcaaggagatgaaggagggctacctcagccaggtggtccacgagatcgccaagctcgtcattgagtacaacgccatcgtcgtcttcgaggacctgaatttcggcttcaagcgcggccggttcaaggtggagaagcaggtctaccagaagcttgagaagatgctgatcgagaagctgaactacctggtgttcaaggacaacgagttcgacaagaccggcggagtgctgcgcgcctaccagctcacggcgcctttcgagacgttcaagaagatgggcaagcagacgggcatcatctactacgtgcccgccggcttcacctctaagatctgcccagtgaccggcttcgttaaccagctgtacccgaagtacgagagcgtgtccaagtcccaggagttcttctccaagttcgacaagatttgttacaacctcgacaagggctacttcgagttttcgttcgactacaagaactttggcgacaaggcggccaaggggaagtggaccatcgcctctttcggcagcaggctcatcaatttccggaactccgacaagaaccacaactgggacacgcgcgaggtgtacccgacgaaggagctggagaagctgctcaaggactactccatcgagtacggccacggcgagtgcatcaaggcggcgatctgcggggagagcgacaagaagttctttgccaagctgaccagcgtgctgaacaccatcctccagatgcggaactccaagaccggcaccgagctggactacctgatctccccggtcgcggacgtcaacgggaacttcttcgactcccgacaggctcccaagaacatgccccaggacgccgacgcgaacggcgcgtaccacatcggcctcaagggcctgatgctgctggggcgcatcaagaacaaccaggagggcaagaagctgaacctcgtgatcaagaacgaggaatacttcgagttcgtgcagaaccgcaacaacggatctggagtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgctaagctgaaggtgaccaagggtggccccctgcccttcgcctgggacatcctgtcccctcatttcacctacggctccaaggcctacgtgaagcaccccgccgacatccccgactacttcaagctgtccttccccgagggcttcaagtgggagcgcgtgatgaactacgaggacggcggcgtggtgaccgtgacccaggactcctccctgcaggacggcgagttcatctacaaggtgaagctgcgcggcaccaacttcccctccgacggccccgtgatgcagaagaagaccatgggctgggaggcctcctccgagcggatgtaccccgaggacggtgccctgaagggcaagatcaagatgaggctgaagctgaaggacggcggccactacacctccgaggtcaagaccacctacaaggccaagaagcccgtgcagctgcccggcgcctacatcgtcgacatcaagttggacatcacctcccacaacgaggactacaccatcgtggaacagtacgaacgcgccgagggccgccactccaccggcggcatggacgagctgtacaagggatctaagaagcgtaggatcaagcaagattagaacccagcggtactcgctgaggaattcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 60 - DNA, 4671 bp, synthetic construct
  misc_feature 1..4671: note = Synthetic polynucleotide.NLS-FnCpf1-Hs-mOR-NLS
  source 1..4671: mol_type = other DNA
ggtagcaaaaagaggcgtatcaagcaggacagcatctaccaggagttcgtcaacaagtattcactgagtaagacactgcggttcgagctgatcccacagggcaagacactggagaacatcaaggcccgaggcctgattctggacgatgagaagcgggcaaaagactataagaaagccaagcagatcattgataaataccaccagttctttatcgaggaaattctgagctccgtgtgcatcagtgaggatctgctgcagaattactcagacgtgtacttcaagctgaagaagagcgacgatgacaacctgcagaaggacttcaagtccgccaaggacaccatcaagaaacagattagcgagtacatcaaggactccgaaaagtttaaaaatctgttcaaccagaatctgatcgatgctaagaaaggccaggagtccgacctgatcctgtggctgaaacagtctaaggacaatgggattgaactgttcaaggctaactccgatatcactgatattgacgaggcactggaaatcatcaagagcttcaagggatggaccacatactttaaaggcttccacgagaaccgcaagaacgtgtactccagcaacgacattcctacctccatcatctaccgaatcgtcgatgacaatctgccaaagttcctggagaacaaggccaaatatgaatctctgaaggacaaagctcccgaggcaattaattacgaacagatcaagaaagatctggctgaggaactgacattcgatatcgactataagactagcgaggtgaaccagagggtcttttccctggacgaggtgtttgaaatcgccaatttcaacaattacctgaaccagtccggcattactaaattcaataccatcattggcgggaagtttgtgaacggggagaataccaagcgcaagggaattaacgaatacatcaatctgtatagccagcagatcaacgacaaaactctgaagaaatacaagatgtctgtgctgttcaaacagatcctgagtgataccgagtccaagtcttttgtcattgataaactggaagatgactcagacgtggtcactaccatgcagagcttttatgagcagatcgccgctttcaagacagtggaggaaaaatctattaaggaaactctgagtctgctgttcgatgacctgaaagcccagaagctggacctgagtaagatctacttcaaaaacgataagagtctgacagacctgtcacagcaggtgtttgatgactattccgtgattgggaccgccgtcctggagtacattacacagcagatcgctccaaagaacctggataatccctctaagaaagagcaggaactgatcgctaagaaaaccgagaaggcaaaatatctgagtctggaaacaattaagctggcactggaggagttcaacaagcacagggatattgacaaacagtgccgctttgaggaaatcctggccaacttcgcagccatccccatgatttttgatgagatcgcccagaacaaagacaatctggctcagatcagtattaagtaccagaaccagggcaagaaagacctgctgcaggcttcagcagaagatgacgtgaaagccatcaaggatctgctggaccagaccaacaatctgctgcacaagctgaaaatcttccatattagtcagtcagaggataaggctaatatcctggataaagacgaacacttctacctggtgttcgaggaatgttacttcgagctggcaaacattgtccccctgtataacaagattaggaactacatcacacagaagccttactctgacgagaagtttaaactgaacttcgaaaatagtaccctggccaacgggtgggataagaacaaggagcctgacaacacagctatcctgttcatcaaggatgacaagtactatctgggagtgatgaataagaaaaacaataagatcttcgatgacaaagccattaaggagaacaaaggggaaggatacaagaaaatcgtgtataagctgctgcccggcgcaaataagatgctgcctaaggtgttcttcagcgccaagagtatcaaattctacaacccatccgaggacatcctgcggattagaaatcactcaacacatactaagaacgggagcccccagaagggatatgagaaatttgagttcaacatcgaggattgcaggaagtttattgacttctacaagcagagcatctccaaacaccctgaatggaaggattttggcttccggttttccgacacacagagatataactctatcgacgagttctaccgcgaggtggaaaatcaggggtataagctgacttttgagaacatttctgaaagttacatcgacagcgtggtcaatcagggaaagctgtacctgttccagatctataacaaagatttttcagcatacagcaagggcagaccaaacctgcatacactgtactggaaggccctgttcgatgagaggaatctgcaggacgtggtctataaactgaacggagaggccgaactgttttaccggaagcagtctattcctaagaaaatcactcacccagctaaggaggccatcgctaacaagaacaaggacaatcctaagaaagagagcgtgttcgaatacgatctgattaaggacaagcggttcaccgaagataagttctttttccattgtccaatcaccattaacttcaagtcaagcggcgctaacaagttcaacgacgagatcaatctgctgctgaaggaaaaagcaaacgatgtgcacatcctgagcattgaccgaggagagcggcatctggcctactataccctggtggatggcaaagggaatatcattaagcaggatacattcaacatcattggcaatgaccggatgaaaaccaactaccacgataaactggctgcaatcgagaaggatagagactcagctaggaaggactggaagaaaatcaacaacattaaggagatgaaggaaggctatctgagccaggtggtccatgagattgcaaagctggtcatcgaatacaatgccattgtggtgttcgaggatctgaacttcggctttaagagggggcgctttaaggtggaaaaacaggtctatcagaagctggagaaaatgctgatcgaaaagctgaattacctggtgtttaaagataacgagttcgacaagaccggaggcgtcctgagagcctaccagctgacagctccctttgaaactttcaagaaaatgggaaaacagacaggcatcatctactatgtgccagccggattcacttccaagatctgccccgtgaccggctttgtcaaccagctgtaccctaaatatgagtcagtgagcaagtcccaggaatttttcagcaagttcgataagatctgttataatctggacaaggggtacttcgagttttccttcgattacaagaacttcggcgacaaggccgctaaggggaaatggaccattgcctccttcggatctcgcctgatcaactttcgaaattccgataaaaaccacaattgggacactagggaggtgtacccaaccaaggagctggaaaagctgctgaaagactactctatcgagtatggacatggcgaatgcatcaaggcagccatctgtggcgagagtgataagaaatttttcgccaagctgacctcagtgctgaatacaatcctgcagatgcggaactcaaagaccgggacagaactggactatctgattagccccgtggctgatgtcaacggaaacttcttcgacagcagacaggcacccaaaaatatgcctcaggatgcagacgccaacggggcctaccacatcgggctgaagggactgatgctgctgggccggatcaagaacaatcaggaggggaagaagctgaacctggtcattaagaacgaggaatacttcgagtttgtccagaatagaaataacggatctggagtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgctaagctgaaggtgaccaagggtggccccctgcccttcgcctgggacatcctgtcccctcatttcacctacggctccaaggcctacgtgaagcaccccgccgacatccccgactacttcaagctgtccttccccgagggcttcaagtgggagcgcgtgatgaactacgaggacggcggcgtggtgaccgtgacccaggactcctccctgcaggacggcgagttcatctacaaggtgaagctgcgcggcaccaacttcccctccgacggccccgtgatgcagaagaagaccatgggctgggaggcctcctccgagcggatgtaccccgaggacggtgccctgaagggcaagatcaagatgaggctgaagctgaaggacggcggccactacacctccgaggtcaagaccacctacaaggccaagaagcccgtgcagctgcccggcgcctacatcgtcgacatcaagttggacatcacctcccacaacgaggactacaccatcgtggaacagtacgaacgcgccgagggccgccactccaccggcggcatggacgagctgtacaagggatctaagaagcgtaggatcaagcaagat

SEQ ID NO: 61 - DNA, 4707 bp, synthetic construct
  misc_feature 1..4707: note = Synthetic polynucleotide.MGSS7H-NLS-FnCpf1-Hs-mOR-NLS
  source 1..4707: mol_type = other DNA
atgggcagcagccatcatcaccaccatcaccatatgggtagcaaaaagaggcgtatcaagcaggacagcatctaccaggagttcgtcaacaagtattcactgagtaagacactgcggttcgagctgatcccacagggcaagacactggagaacatcaaggcccgaggcctgattctggacgatgagaagcgggcaaaagactataagaaagccaagcagatcattgataaataccaccagttctttatcgaggaaattctgagctccgtgtgcatcagtgaggatctgctgcagaattactcagacgtgtacttcaagctgaagaagagcgacgatgacaacctgcagaaggacttcaagtccgccaaggacaccatcaagaaacagattagcgagtacatcaaggactccgaaaagtttaaaaatctgttcaaccagaatctgatcgatgctaagaaaggccaggagtccgacctgatcctgtggctgaaacagtctaaggacaatgggattgaactgttcaaggctaactccgatatcactgatattgacgaggcactggaaatcatcaagagcttcaagggatggaccacatactttaaaggcttccacgagaaccgcaagaacgtgtactccagcaacgacattcctacctccatcatctaccgaatcgtcgatgacaatctgccaaagttcctggagaacaaggccaaatatgaatctctgaaggacaaagctcccgaggcaattaattacgaacagatcaagaaagatctggctgaggaactgacattcgatatcgactataagactagcgaggtgaaccagagggtcttttccctggacgaggtgtttgaaatcgccaatttcaacaattacctgaaccagtccggcattactaaattcaataccatcattggcgggaagtttgtgaacggggagaataccaagcgcaagggaattaacgaatacatcaatctgtatagccagcagatcaacgacaaaactctgaagaaatacaagatgtctgtgctgttcaaacagatcctgagtgataccgagtccaagtcttttgtcattgataaactggaagatgactcagacgtggtcactaccatgcagagcttttatgagcagatcgccgctttcaagacagtggaggaaaaatctattaaggaaactctgagtctgctgttcgatgacctgaaagcccagaagctggacctgagtaagatctacttcaaaaacgataagagtctgacagacctgtcacagcaggtgtttgatgactattccgtgattgggaccgccgtcctggagtacattacacagcagatcgctccaaagaacctggataatccctctaagaaagagcaggaactgatcgctaagaaaaccgagaaggcaaaatatctgagtctggaaacaattaagctggcactggaggagttcaacaagcacagggatattgacaaacagtgccgctttgaggaaatcctggccaacttcgcagccatccccatgatttttgatgagatcgcccagaacaaagacaatctggctcagatcagtattaagtaccagaaccagggcaagaaagacctgctgcaggcttcagcagaagatgacgtgaaagccatcaaggatctgctggaccagaccaacaatctgctgcacaagctgaaaatcttccatattagtcagtcagaggataaggctaatatcctggataaagacgaacacttctacctggtgttcgaggaatgttacttcgagctggcaaacattgtccccctgtataacaagattaggaactacatcacacagaagccttactctgacgagaagtttaaactgaacttcgaaaatagtaccctggccaacgggtgggataagaacaaggagcctgacaacacagctatcctgttcatcaaggatgacaagtactatctgggagtgatgaataagaaaaacaataagatcttcgatgacaaagccattaaggagaacaaaggggaaggatacaagaaaatcgtgtataagctgctgcccggcgcaaataagatgctgcctaaggtgttcttcagcgccaagagtatcaaattctacaacccatccgaggacatcctgcggattagaaatcactcaacacatactaagaacgggagcccccagaagggatatgagaaatttgagttcaacatcgaggattgcaggaagtttattgacttctacaagcagagcatctccaaacaccctgaatggaaggattttggcttccggttttccgacacacagagatataactctatcgacgagttctaccgcgaggtggaaaatcaggggtataagctgacttttgagaacatttctgaaagttacatcgacagcgtggtcaatcagggaaagctgtacctgttccagatctataacaaagatttttcagcatacagcaagggcagaccaaacctgcatacactgtactggaaggccctgttcgatgagaggaatctgcaggacgtggtctataaactgaacggagaggccgaactgttttaccggaagcagtctattcctaagaaaatcactcacccagctaaggaggccatcgctaacaagaacaaggacaatcctaagaaagagagcgtgttcgaatacgatctgattaaggacaagcggttcaccgaagataagttctttttccattgtccaatcaccattaacttcaagtcaagcggcgctaacaagttcaacgacgagatcaatctgctgctgaaggaaaaagcaaacgatgtgcacatcctgagcattgaccgaggagagcggcatctggcctactataccctggtggatggcaaagggaatatcattaagcaggatacattcaacatcattggcaatgaccggatgaaaaccaactaccacgataaactggctgcaatcgagaaggatagagactcagctaggaaggactggaagaaaatcaacaacattaaggagatgaaggaaggctatctgagccaggtggtccatgagattgcaaagctggtcatcgaatacaatgccattgtggtgttcgaggatctgaacttcggctttaagagggggcgctttaaggtggaaaaacaggtctatcagaagctggagaaaatgctgatcgaaaagctgaattacctggtgtttaaagataacgagttcgacaagaccggaggcgtcctgagagcctaccagctgacagctccctttgaaactttcaagaaaatgggaaaacagacaggcatcatctactatgtgccagccggattcacttccaagatctgccccgtgaccggctttgtcaaccagctgtaccctaaatatgagtcagtgagcaagtcccaggaatttttcagcaagttcgataagatctgttataatctggacaaggggtacttcgagttttccttcgattacaagaacttcggcgacaaggccgctaaggggaaatggaccattgcctccttcggatctcgcctgatcaactttcgaaattccgataaaaaccacaattgggacactagggaggtgtacccaaccaaggagctggaaaagctgctgaaagactactctatcgagtatggacatggcgaatgcatcaaggcagccatctgtggcgagagtgataagaaatttttcgccaagctgacctcagtgctgaatacaatcctgcagatgcggaactcaaagaccgggacagaactggactatctgattagccccgtggctgatgtcaacggaaacttcttcgacagcagacaggcacccaaaaatatgcctcaggatgcagacgccaacggggcctaccacatcgggctgaagggactgatgctgctgggccggatcaagaacaatcaggaggggaagaagctgaacctggtcattaagaacgaggaatacttcgagtttgtccagaatagaaataacggatctggagtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgctaagctgaaggtgaccaagggtggccccctgcccttcgcctgggacatcctgtcccctcatttcacctacggctccaaggcctacgtgaagcaccccgccgacatccccgactacttcaagctgtccttccccgagggcttcaagtgggagcgcgtgatgaactacgaggacggcggcgtggtgaccgtgacccaggactcctccctgcaggacggcgagttcatctacaaggtgaagctgcgcggcaccaacttcccctccgacggccccgtgatgcagaagaagaccatgggctgggaggcctcctccgagcggatgtaccccgaggacggtgccctgaagggcaagatcaagatgaggctgaagctgaaggacggcggccactacacctccgaggtcaagaccacctacaaggccaagaagcccgtgcagctgcccggcgcctacatcgtcgacatcaagttggacatcacctcccacaacgaggactacaccatcgtggaacagtacgaacgcgccgagggccgccactccaccggcggcatggacgagctgtacaagggatctaagaagcgtaggatcaagcaagat

SEQ ID NO: 62 - DNA, 6462 bp, synthetic construct
  misc_feature 1..6462: note = Synthetic polynucleotide.Expression cassette comprising 35Spromoter cassette, MGSS7H-NLS-FnCpf1-Hs-mOr-NLS and NOStranscription termination sequence
  source 1..6462: mol_type = other DNA
ggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacaagctgactctagcagatctaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcctctagaggatccaggagcaaccatgggcagcagccatcatcaccaccatcaccatatgggtagcaaaaagaggcgtatcaagcaggacagcatctaccaggagttcgtcaacaagtattcactgagtaagacactgcggttcgagctgatcccacagggcaagacactggagaacatcaaggcccgaggcctgattctggacgatgagaagcgggcaaaagactataagaaagccaagcagatcattgataaataccaccagttctttatcgaggaaattctgagctccgtgtgcatcagtgaggatctgctgcagaattactcagacgtgtacttcaagctgaagaagagcgacgatgacaacctgcagaaggacttcaagtccgccaaggacaccatcaagaaacagattagcgagtacatcaaggactccgaaaagtttaaaaatctgttcaaccagaatctgatcgatgctaagaaaggccaggagtccgacctgatcctgtggctgaaacagtctaaggacaatgggattgaactgttcaaggctaactccgatatcactgatattgacgaggcactggaaatcatcaagagcttcaagggatggaccacatactttaaaggcttccacgagaaccgcaagaacgtgtactccagcaacgacattcctacctccatcatctaccgaatcgtcgatgacaatctgccaaagttcctggagaacaaggccaaatatgaatctctgaaggacaaagctcccgaggcaattaattacgaacagatcaagaaagatctggctgaggaactgacattcgatatcgactataagactagcgaggtgaaccagagggtcttttccctggacgaggtgtttgaaatcgccaatttcaacaattacctgaaccagtccggcattactaaattcaataccatcattggcgggaagtttgtgaacggggagaataccaagcgcaagggaattaacgaatacatcaatctgtatagccagcagatcaacgacaaaactctgaagaaatacaagatgtctgtgctgttcaaacagatcctgagtgataccgagtccaagtcttttgtcattgataaactggaagatgactcagacgtggtcactaccatgcagagcttttatgagcagatcgccgctttcaagacagtggaggaaaaatctattaaggaaactctgagtctgctgttcgatgacctgaaagcccagaagctggacctgagtaagatctacttcaaaaacgataagagtctgacagacctgtcacagcaggtgtttgatgactattccgtgattgggaccgccgtcctggagtacattacacagcagatcgctccaaagaacctggataatccctctaagaaagagcaggaactgatcgctaagaaaaccgagaaggcaaaatatctgagtctggaaacaattaagctggcactggaggagttcaacaagcacagggatattgacaaacagtgccgctttgaggaaatcctggccaacttcgcagccatccccatgatttttgatgagatcgcccagaacaaagacaatctggctcagatcagtattaagtaccagaaccagggcaagaaagacctgctgcaggcttcagcagaagatgacgtgaaagccatcaaggatctgctggaccagaccaacaatctgctgcacaagctgaaaatcttccatattagtcagtcagaggataaggctaatatcctggataaagacgaacacttctacctggtgttcgaggaatgttacttcgagctggcaaacattgtccccctgtataacaagattaggaactacatcacacagaagccttactctgacgagaagtttaaactgaacttcgaaaatagtaccctggccaacgggtgggataagaacaaggagcctgacaacacagctatcctgttcatcaaggatgacaagtactatctgggagtgatgaataagaaaaacaataagatcttcgatgacaaagccattaaggagaacaaaggggaaggatacaagaaaatcgtgtataagctgctgcccggcgcaaataagatgctgcctaaggtgttcttcagcgccaagagtatcaaattctacaacccatccgaggacatcctgcggattagaaatcactcaacacatactaagaacgggagcccccagaagggatatgagaaatttgagttcaacatcgaggattgcaggaagtttattgacttctacaagcagagcatctccaaacaccctgaatggaaggattttggcttccggttttccgacacacagagatataactctatcgacgagttctaccgcgaggtggaaaatcaggggtataagctgacttttgagaacatttctgaaagttacatcgacagcgtggtcaatcagggaaagctgtacctgttccagatctataacaaagatttttcagcatacagcaagggcagaccaaacctgcatacactgtactggaaggccctgttcgatgagaggaatctgcaggacgtggtctataaactgaacggagaggccgaactgttttaccggaagcagtctattcctaagaaaatcactcacccagctaaggaggccatcgctaacaagaacaaggacaatcctaagaaagagagcgtgttcgaatacgatctgattaaggacaagcggttcaccgaagataagttctttttccattgtccaatcaccattaacttcaagtcaagcggcgctaacaagttcaacgacgagatcaatctgctgctgaaggaaaaagcaaacgatgtgcacatcctgagcattgaccgaggagagcggcatctggcctactataccctggtggatggcaaagggaatatcattaagcaggatacattcaacatcattggcaatgaccggatgaaaaccaactaccacgataaactggctgcaatcgagaaggatagagactcagctaggaaggactggaagaaaatcaacaacattaaggagatgaaggaaggctatctgagccaggtggtccatgagattgcaaagctggtcatcgaatacaatgccattgtggtgttcgaggatctgaacttcggctttaagagggggcgctttaaggtggaaaaacaggtctatcagaagctggagaaaatgctgatcgaaaagctgaattacctggtgtttaaagataacgagttcgacaagaccggaggcgtcctgagagcctaccagctgacagctccctttgaaactttcaagaaaatgggaaaacagacaggcatcatctactatgtgccagccggattcacttccaagatctgccccgtgaccggctttgtcaaccagctgtaccctaaatatgagtcagtgagcaagtcccaggaatttttcagcaagttcgataagatctgttataatctggacaaggggtacttcgagttttccttcgattacaagaacttcggcgacaaggccgctaaggggaaatggaccattgcctccttcggatctcgcctgatcaactttcgaaattccgataaaaaccacaattgggacactagggaggtgtacccaaccaaggagctggaaaagctgctgaaagactactctatcgagtatggacatggcgaatgcatcaaggcagccatctgtggcgagagtgataagaaatttttcgccaagctgacctcagtgctgaatacaatcctgcagatgcggaactcaaagaccgggacagaactggactatctgattagccccgtggctgatgtcaacggaaacttcttcgacagcagacaggcacccaaaaatatgcctcaggatgcagacgccaacggggcctaccacatcgggctgaagggactgatgctgctgggccggatcaagaacaatcaggaggggaagaagctgaacctggtcattaagaacgaggaatacttcgagtttgtccagaatagaaataacggatctggagtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgctaagctgaaggtgaccaagggtggccccctgcccttcgcctgggacatcctgtcccctcatttcacctacggctccaaggcctacgtgaagcaccccgccgacatccccgactacttcaagctgtccttccccgagggcttcaagtgggagcgcgtgatgaactacgaggacggcggcgtggtgaccgtgacccaggactcctccctgcaggacggcgagttcatctacaaggtgaagctgcgcggcaccaacttcccctccgacggccccgtgatgcagaagaagaccatgggctgggaggcctcctccgagcggatgtaccccgaggacggtgccctgaagggcaagatcaagatgaggctgaagctgaaggacggcggccactacacctccgaggtcaagaccacctacaaggccaagaagcccgtgcagctgcccggcgcctacatcgtcgacatcaagttggacatcacctcccacaacgaggactacaccatcgtggaacagtacgaacgcgccgagggccgccactccaccggcggcatggacgagctgtacaagggatctaagaagcgtaggatcaagcaagattagaacccagcggtactcgctgaggaattcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 63 - DNA, 3960 bp, synthetic construct
  misc_feature 1..3960: note = Synthetic polynucleotide.NLS-FnCpf1-CO3-NLS
  source 1..3960: mol_type = other DNA
ggtagcaaaaagaggcgtatcaagcaggacatgagcatctaccaggagttcgtgaacaagtacagcctgtcgaagaccctccggttcgagctgatccctcaggggaagacgctggagaacatcaaggcgcgcggcctcatcctcgacgacgagaagcgggcgaaggactacaaaaaggcaaagcagatcatcgacaagtaccaccaattctttattgaggagatcctcagctccgtctgcatcagcgaggacttgctccagaactactccgacgtctatttcaagctcaagaagtcagacgacgacaacctccagaaggacttcaagtccgcgaaggacacgatcaaaaagcagatcagcgagtacatcaaggactccgagaagttcaagaacctcttcaaccagaacctgatcgacgccaagaagggccgggaatcggacctcatcctgtggctcaagcagtcgaaggacaacggcatcgagctgttcaaggccaactccgacatcaccgacatcgacgaggcgctggagatcatcaagtcgttcaaggggtggaccacctacttcaagggcttccacgagaaccgcaagaacgtttactccagcaacgacatccccacctcgatcatctaccggatcgtggacgacaacctgcccaagttcctggagaacaaggccaagtacgagtccctcaaggacaaggccccggaggcgatcaactacgagcagattaaaaaggaccttgctgaggagctgaccttcgacatcgactacaagacctccgaggtgaaccagcgggtgttcagcctcgacgaggtgttcgagatcgccaacttcaacaactacctgaaccagtccgggatcaccaagttcaacacgatcatcggcgggaagttcgtcaacggcgagaacacgaagcggaagggcatcaacgagtacatcaacctctacagccagcagatcaacgacaagaccctcaaaaaatacaaaatgtcggtcctgttcaagcagatcctgtccgacaccgagtccaagagcttcgtcatcgacaagctggaggacgactccgacgtcgtgaccaccatgcagtccttctacgagcagatcgccgccttcaagaccgtggaggagaagtccatcaaggagaccctcagcctcctcttcgacgacctcaaggcgcagaagctggacctctccaagatatacttcaagaacgacaagagcctcaccgacctgtcccagcaagtattcgacgactacagcgtgatcgggacggcggtgctggagtacatcacccagcagatagcgcccaagaacctggacaacccctccaagaaagaacaggagctgattgctaaaaagaccgaaaaggctaagtacctgtccctggagaccatcaagctcgcgctggaggagttcaacaagcaccgggacatcgacaagcagtgccggttcgaggagatcctagcaaacttcgccgcgatccccatgatcttcgacgagatcgcccagaacaaggacaacctggcccagatcagcatcaagtaccagaaccagggcaagaaggaccttcttcaagctagtgccgaggacgacgtgaaggcgattaaggatctgctcgaccagaccaacaacctgctccacaagctcaagatattccacatctcccagtccgaggacaaggccaacatcctggacaaggacgagcacttctacctggtgttcgaggagtgctacttcgagctggccaacatcgtgccgctgtacaacaagatccggaactacatcacccagaagccctactccgacgagaagttcaagctgaacttcgagaactccaccctggcgaacgggtgggacaagaacaaggagcccgacaacacggccatcctcttcatcaaggacgacaaatattatctgggcgtcatgaacaaaaagaacaacaagatattcgatgacaaggcgatcaaggagaacaagggcgagggctacaagaaaatagtatataaactactgcccggcgcgaacaagatgctcccgaaggtgttttttagtgcaaagtctattaagttctacaaccccagcgaggacatcctccgcatccggaaccacagcacgcacaccaagaacggcagcccacagaagggctacgagaagttcgagttcaacatcgaggactgccgcaagttcatcgacttctacaagcagtccatctccaagcaccccgagtggaaggacttcgggttccggttcagcgacacccagcgctacaacagcatcgacgagttctaccgggaggtcgagaaccaggggtacaagctgacgttcgagaacatctccgagagctacatcgacagcgtggtgaaccaggggaagctgtacctgtttcagatatacaacaaggacttctcagcctacagcaaggggcggccgaacctgcacaccctgtactggaaggccctgttcgacgagcggaacctccaggacgtcgtgtacaagctcaacggcgaggcggagctgttctaccggaagcagtccatccccaaaaagattactcaccccgcgaaggaggccatcgccaacaagaacaaggacaaccccaaaaaggaatcagtgttcgagtacgacctcatcaaggacaagcgcttcaccgaggacaaattcttctttcactgcccgatcacgatcaacttcaagtcctccggggcgaacaagttcaacgacgagatcaacctgctgctcaaggagaaggccaacgacgtgcacatcctcagcatcgaccggggcgagcgccacctggcctactacaccctggtggacgggaagggcaacatcataaagcaagataccttcaacatcatcgggaacgaccggatgaagacgaactaccacgacaagctggcggccatcgagaaggaccgggacagcgcccgcaaggactggaaaaagataaacaacattaaggagatgaaggagggctacctgtcccaggtggtccacgagatcgccaagctcgtcatcgagtacaacgccatcgtcgtgttcgaggacttgaacttcgggttcaagcggggccggttcaaggtggagaaacaagtctatcaaaagctggagaagatgctcatcgagaagctcaactacctcgtgttcaaggacaacgagttcgacaagaccggcggcgtcctgcgggcctaccagctcaccgcgccgttcgagacgttcaagaagatggggaagcagacggggatcatctactacgtccccgccgggttcaccagcaagatatgcccggtcacggggttcgtcaaccagctctaccccaagtacgagtcggtgagcaagagccaggagttcttcagcaagttcgacaagatctgctacaacctggacaagggctacttcgagttctcgttcgactacaagaacttcggggacaaggcggcgaagggcaagtggaccatcgccagcttcggctcccgcctcatcaacttccggaactcggacaagaaccacaactgggacacccgcgaggtgtaccccacgaaggagctggagaagctgctcaaggactacagcatcgagtacgggcacggcgagtgcatcaaggccgccatctgcggggagtccgacaagaaattctttgccaagctgacctccgtgctgaacaccatcctccagatgcggaacagcaagaccgggaccgagctggactacctgatctccccggtcgccgacgtcaacggcaacttcttcgattctcgccaggctcccaagaacatgccccaggacgccgacgccaacggggcctaccacatcgggctcaagggcctcatgctgctcgggcggatcaagaacaaccaggagggcaagaaactcaacctggtcatcaagaacgaggagtactttgagttcgtccagaaccggaacaacggatctaagaagcgtaggatcaagcaagat

SEQ ID NO: 64 - DNA, 530 bp, Oryza sativa
  source 1..530: mol_type = unassigned DNA
atccaagtaaattaagttggatcagtagagatgcatggttggtgttctcatgtggtctgaatctgtgttctgtttcatcttttgtgtaagtgtgcaacttcgattctgtaaaacttggcaagtagatgaacgatgtcatttatatgcgtgtagcttgtcaaaatacaaagtagaaacctgtatctgaataagatggcattcatgaaataatattatcgattgaacgaacgaagcaacagaatgatcttccaaattgaagcaaaggaattgatggacaacgagttgctgtactttggagctgtaaatctgtagtacttctcaacgatgctatggaccgtgggccgtccataatcgctgggtggaatgagcccactgtccatgtgcttctgcatttggcccgtgtacagcccatataatgtcgaaaaacctaatacgcgcgcgccgccggcgcgcgtttcggacccagaacgcctagcgccgctgcgccgacgtgcgcgcgtctccactttttttttcgttttctttccatt

SEQ ID NO: 65 - DNA, 6530 bp, synthetic construct
  misc_feature 1..6530: note = Synthetic polynucleotide.Expression cassette comprising Zea maysUbiquitin promoter cassette, NLS-FnCpf1-CO3-NLS and an Oryzasativa transcription termination sequence
  source 1..6530: mol_type = other DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgttgggtgatacttctgcagcggaccgacggtaccatgggtagcaaaaagaggcgtatcaagcaggacatgagcatctaccaggagttcgtgaacaagtacagcctgtcgaagaccctccggttcgagctgatccctcaggggaagacgctggagaacatcaaggcgcgcggcctcatcctcgacgacgagaagcgggcgaaggactacaaaaaggcaaagcagatcatcgacaagtaccaccaattctttattgaggagatcctcagctccgtctgcatcagcgaggacttgctccagaactactccgacgtctatttcaagctcaagaagtcagacgacgacaacctccagaaggacttcaagtccgcgaaggacacgatcaaaaagcagatcagcgagtacatcaaggactccgagaagttcaagaacctcttcaaccagaacctgatcgacgccaagaagggccgggaatcggacctcatcctgtggctcaagcagtcgaaggacaacggcatcgagctgttcaaggccaactccgacatcaccgacatcgacgaggcgctggagatcatcaagtcgttcaaggggtggaccacctacttcaagggcttccacgagaaccgcaagaacgtttactccagcaacgacatccccacctcgatcatctaccggatcgtggacgacaacctgcccaagttcctggagaacaaggccaagtacgagtccctcaaggacaaggccccggaggcgatcaactacgagcagattaaaaaggaccttgctgaggagctgaccttcgacatcgactacaagacctccgaggtgaaccagcgggtgttcagcctcgacgaggtgttcgagatcgccaacttcaacaactacctgaaccagtccgggatcaccaagttcaacacgatcatcggcgggaagttcgtcaacggcgagaacacgaagcggaagggcatcaacgagtacatcaacctctacagccagcagatcaacgacaagaccctcaaaaaatacaaaatgtcggtcctgttcaagcagatcctgtccgacaccgagtccaagagcttcgtcatcgacaagctggaggacgactccgacgtcgtgaccaccatgcagtccttctacgagcagatcgccgccttcaagaccgtggaggagaagtccatcaaggagaccctcagcctcctcttcgacgacctcaaggcgcagaagctggacctctccaagatatacttcaagaacgacaagagcctcaccgacctgtcccagcaagtattcgacgactacagcgtgatcgggacggcggtgctggagtacatcacccagcagatagcgcccaagaacctggacaacccctccaagaaagaacaggagctgattgctaaaaagaccgaaaaggctaagtacctgtccctggagaccatcaagctcgcgctggaggagttcaacaagcaccgggacatcgacaagcagtgccggttcgaggagatcctagcaaacttcgccgcgatccccatgatcttcgacgagatcgcccagaacaaggacaacctggcccagatcagcatcaagtaccagaaccagggcaagaaggaccttcttcaagctagtgccgaggacgacgtgaaggcgattaaggatctgctcgaccagaccaacaacctgctccacaagctcaagatattccacatctcccagtccgaggacaaggccaacatcctggacaaggacgagcacttctacctggtgttcgaggagtgctacttcgagctggccaacatcgtgccgctgtacaacaagatccggaactacatcacccagaagccctactccgacgagaagttcaagctgaacttcgagaactccaccctggcgaacgggtgggacaagaacaaggagcccgacaacacggccatcctcttcatcaaggacgacaaatattatctgggcgtcatgaacaaaaagaacaacaagatattcgatgacaaggcgatcaaggagaacaagggcgagggctacaagaaaatagtatataaactactgcccggcgcgaacaagatgctcccgaaggtgttttttagtgcaaagtctattaagttctacaaccccagcgaggacatcctccgcatccggaaccacagcacgcacaccaagaacggcagcccacagaagggctacgagaagttcgagttcaacatcgaggactgccgcaagttcatcgacttctacaagcagtccatctccaagcaccccgagtggaaggacttcgggttccggttcagcgacacccagcgctacaacagcatcgacgagttctaccgggaggtcgagaaccaggggtacaagctgacgttcgagaacatctccgagagctacatcgacagcgtggtgaaccaggggaagctgtacctgtttcagatatacaacaaggacttctcagcctacagcaaggggcggccgaacctgcacaccctgtactggaaggccctgttcgacgagcggaacctccaggacgtcgtgtacaagctcaacggcgaggcggagctgttctaccggaagcagtccatccccaaaaagattactcaccccgcgaaggaggccatcgccaacaagaacaaggacaaccccaaaaaggaatcagtgttcgagtacgacctcatcaaggacaagcgcttcaccgaggacaaattcttctttcactgcccgatcacgatcaacttcaagtcctccggggcgaacaagttcaacgacgagatcaacctgctgctcaaggagaaggccaacgacgtgcacatcctcagcatcgaccggggcgagcgccacctggcctactacaccctggtggacgggaagggcaacatcataaagcaagataccttcaacatcatcgggaacgaccggatgaagacgaactaccacgacaagctggcggccatcgagaaggaccgggacagcgcccgcaaggactggaaaaagataaacaacattaaggagatgaaggagggctacctgtcccaggtggtccacgagatcgccaagctcgtcatcgagtacaacgccatcgtcgtgttcgaggacttgaacttcgggttcaagcggggccggttcaaggtggagaaacaagtctatcaaaagctggagaagatgctcatcgagaagctcaactacctcgtgttcaaggacaacgagttcgacaagaccggcggcgtcctgcgggcctaccagctcaccgcgccgttcgagacgttcaagaagatggggaagcagacggggatcatctactacgtccccgccgggttcaccagcaagatatgcccggtcacggggttcgtcaaccagctctaccccaagtacgagtcggtgagcaagagccaggagttcttcagcaagttcgacaagatctgctacaacctggacaagggctacttcgagttctcgttcgactacaagaacttcggggacaaggcggcgaagggcaagtggaccatcgccagcttcggctcccgcctcatcaacttccggaactcggacaagaaccacaactgggacacccgcgaggtgtaccccacgaaggagctggagaagctgctcaaggactacagcatcgagtacgggcacggcgagtgcatcaaggccgccatctgcggggagtccgacaagaaattctttgccaagctgacctccgtgctgaacaccatcctccagatgcggaacagcaagaccgggaccgagctggactacctgatctccccggtcgccgacgtcaacggcaacttcttcgattctcgccaggctcccaagaacatgccccaggacgccgacgccaacggggcctaccacatcgggctcaagggcctcatgctgctcgggcggatcaagaacaaccaggagggcaagaaactcaacctggtcatcaagaacgaggagtactttgagttcgtccagaaccggaacaacggatctaagaagcgtaggatcaagcaagattgattaattaagggcccatccaagtaaattaagttggatcagtagagatgcatggttggtgttctcatgtggtctgaatctgtgttctgtttcatcttttgtgtaagtgtgcaacttcgattctgtaaaacttggcaagtagatgaacgatgtcatttatatgcgtgtagcttgtcaaaatacaaagtagaaacctgtatctgaataagatggcattcatgaaataatattatcgattgaacgaacgaagcaacagaatgatcttccaaattgaagcaaaggaattgatggacaacgagttgctgtactttggagctgtaaatctgtagtacttctcaacgatgctatggaccgtgggccgtccataatcgctgggtggaatgagcccactgtccatgtgcttctgcatttggcccgtgtacagcccatataatgtcgaaaaacctaatacgcgcgcgccgccggcgcgcgtttcggacccagaacgcctagcgccgctgcgccgacgtgcgcgcgtctccactttttttttcgttttctttccatt

SEQ ID NO: 66 - DNA, 3960 bp, synthetic construct
  misc_feature 1..3960: note = Synthetic polynucleotide.NLS-FnCpf1-CO4-NLS
  source 1..3960: mol_type = other DNA
ggtagcaaaaagaggcgtatcaagcaggacatgagcatctaccaggagttcgtgaacaagtacagcctgagcaagaccctgcgcttcgagctgattcctcaggggaagaccctggagaacatcaaggcgcgcggcctgatcctggacgacgagaagcgggcgaaggactacaaaaaggcgaagcagatcatcgacaagtaccatcagtttttcatcgaggagattctcagctccgtgtgcatcagcgaagacctgctccagaactacagcgacgtttacttcaagctcaagaaatcggacgacgacaacctccagaaggacttcaagagcgcgaaggacacgattaagaaacagatcagcgagtacatcaaggactccgagaagttcaagaacctgttcaaccagaacctcatcgacgccaagaaaggccaagagtcggacctcatcctctggctcaagcagagcaaggacaacggcatcgagctgttcaaggcgaacagcgacatcaccgacatcgacgaggcgctggagatcatcaagtcgttcaagggctggacgacctacttcaagggcttccacgagaaccgcaagaatgtctactcgagcaacgacatccccacgtcgatcatctaccgcatcgtggacgacaacctcccgaagttcctggagaacaaggcgaagtacgagagcctcaaggacaaggccccggaggccatcaactacgagcagatcaaaaaggatttggctgaggagctgacgttcgacatcgactacaagacctccgaggtgaaccagcgcgtcttcagcctcgacgaggtgttcgagatcgccaacttcaacaactacctcaaccagtcgggcatcaccaagttcaacaccatcatcggcgggaagttcgtcaacggcgagaacacgaagcgcaaggggatcaacgagtacatcaacctgtacagccagcagatcaacgacaagaccctcaagaagtataaaatgtcggtgctgttcaagcagatcctctcggacaccgagagcaagtcgttcgtcatcgacaagctggaggacgacagcgacgtggtgaccaccatgcagagcttctacgagcagatcgcggccttcaagaccgtcgaggagaagtcgatcaaggagacgctcagcctgctgttcgacgacctcaaggcccagaagctcgacctgtccaagatatactttaagaacgacaagagcctcacggacctgtcccagcaggtattcgacgactacagcgtgatcgggacggccgtgctggagtacatcacccaacagatcgcgcccaagaacctggacaacccgtccaagaaggaacaagagctaatcgccaaaaagactgagaaggcgaagtacctgtcgctggagacgatcaagctcgcgcttgaggagtttaacaagcaccgcgacatcgacaagcagtgccggttcgaggagatcctggccaacttcgcggcgatcccgatgatcttcgacgagatcgcccagaacaaggacaacctggcgcagatcagcatcaagtaccagaaccagggcaaaaaagacttgctccaagctagtgcggaggacgacgtgaaggcgattaaggatctgctggaccagactaacaatctgctgcacaagctcaagatctttcacatctctcagtcggaggacaaggcgaacatcctggacaaggacgagcacttctacctagtgttcgaggagtgctacttcgagctggcgaacatcgtgcccctgtacaacaagatccggaactacatcacccagaagccctacagcgacgagaagttcaagctgaacttcgagaacagcacgctggcgaacgggtgggacaagaacaaggagcccgacaacaccgccatcctgttcatcaaggacgacaaatattacctcggcgtcatgaacaaaaagaataacaagatattcgatgacaaggcgatcaaggagaacaagggcgagggctacaagaagatcgtatataaactcctgccgggagcgaacaagatgctcccgaaggttttctttagtgccaagtccatcaagttctacaaccccagcgaggacatcctccgcatccggaaccactccacccacaccaagaacggctcgccgcagaagggctacgagaagttcgagttcaacatcgaggactgccgcaagttcatcgacttctacaagcagtccatctccaagcacccggagtggaaggacttcgggttccggttctccgacacgcagcgctacaactccatcgacgagttctaccgggaggtggagaaccagggctacaagctgacgttcgagaacatctcggagtcctacatcgactccgtggtcaaccagggcaagctgtacctcttccagatatacaataaggacttctccgcctacagcaaggggcggcccaacctccacaccctgtactggaaggcgctcttcgacgagcggaacctccaggacgtcgtgtacaagctgaacggcgaggcggagctgttctaccgcaagcagagcatccccaagaagatcacgcaccccgcgaaggaggccatcgccaacaagaacaaggacaaccccaagaaggaatcggtcttcgagtacgacctcatcaaggacaagcggttcacggaggacaaattctttttccactgcccgatcactattaacttcaagtccagcggcgcgaacaagttcaacgacgagatcaacctgctcctcaaggagaaggcgaacgacgtgcacatcctcagcatcgaccggggcgagcgccacctcgcctactacacgctggtggacgggaagggcaacatcatcaagcaagacaccttcaacatcatcggcaacgaccggatgaagaccaactaccacgacaagctggccgccatcgagaaggaccgcgactcggcccgcaaggactggaaaaagatcaacaatatcaaggagatgaaggagggctacctgagccaagttgtccacgagatcgccaagctggtgatcgagtacaacgccatcgtcgtgttcgaagacctgaacttcggcttcaagcgcggccggttcaaggtcgagaaacaagtctatcagaaacttgagaagatgctgatcgagaagctgaactacctcgtcttcaaggacaacgagttcgacaagaccggcggcgtcctccgcgcgtaccagctcaccgcgccgttcgagacgttcaagaaaatgggcaagcagaccggcatcatctactacgtgcccgccgggttcacgagcaaaatatgtcccgtgaccggcttcgtcaaccagctctaccccaagtacgagtccgtgtcgaagtcccaggaattcttcagcaagttcgacaagatatgctacaacctggacaagggctacttcgagttctccttcgactacaagaacttcggggacaaggcggcgaaggggaagtggaccatcgcctcgttcgggtcgcgcctcatcaacttccggaacagcgacaagaaccacaactgggacacccgcgaggtgtacccgacgaaggagctggagaagctcctcaaggactacagcatcgagtacggccacggggagtgcatcaaggcggccatctgcggcgagtcggacaaaaagttctttgccaaactcacctcggtcctcaacaccatcctccagatgcggaacagcaagacgggcacggagctggactacctcatcagcccggtggccgacgtgaacggcaatttctttgactcacgccaggcccctaagaacatgccccaggacgccgacgccaacggcgcgtaccacatcggcctcaagggcctgatgctgctcggccggatcaagaacaaccaggagggcaagaagctcaacctggtcatcaagaacgaggagtatttcgagttcgtccagaaccgcaacaacggatctaagaagcgtaggatcaagcaagat

SEQ ID NO: 67 - DNA, 6530 bp, synthetic construct
  misc_feature 1..6530: note = Synthetic polynucleotide.Expression cassette comprising Zea maysUbiquitin promoter cassette, NLS-FnCpf1-CO4-NLS and an Oryzasativa transcription termination sequence
  source 1..6530: mol_type = other DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgttgggtgatacttctgcagcggaccgacggtaccatgggtagcaaaaagaggcgtatcaagcaggacatgagcatctaccaggagttcgtgaacaagtacagcctgagcaagaccctgcgcttcgagctgattcctcaggggaagaccctggagaacatcaaggcgcgcggcctgatcctggacgacgagaagcgggcgaaggactacaaaaaggcgaagcagatcatcgacaagtaccatcagtttttcatcgaggagattctcagctccgtgtgcatcagcgaagacctgctccagaactacagcgacgtttacttcaagctcaagaaatcggacgacgacaacctccagaaggacttcaagagcgcgaaggacacgattaagaaacagatcagcgagtacatcaaggactccgagaagttcaagaacctgttcaaccagaacctcatcgacgccaagaaaggccaagagtcggacctcatcctctggctcaagcagagcaaggacaacggcatcgagctgttcaaggcgaacagcgacatcaccgacatcgacgaggcgctggagatcatcaagtcgttcaagggctggacgacctacttcaagggcttccacgagaaccgcaagaatgtctactcgagcaacgacatccccacgtcgatcatctaccgcatcgtggacgacaacctcccgaagttcctggagaacaaggcgaagtacgagagcctcaaggacaaggccccggaggccatcaactacgagcagatcaaaaaggatttggctgaggagctgacgttcgacatcgactacaagacctccgaggtgaaccagcgcgtcttcagcctcgacgaggtgttcgagatcgccaacttcaacaactacctcaaccagtcgggcatcaccaagttcaacaccatcatcggcgggaagttcgtcaacggcgagaacacgaagcgcaaggggatcaacgagtacatcaacctgtacagccagcagatcaacgacaagaccctcaagaagtataaaatgtcggtgctgttcaagcagatcctctcggacaccgagagcaagtcgttcgtcatcgacaagctggaggacgacagcgacgtggtgaccaccatgcagagcttctacgagcagatcgcggccttcaagaccgtcgaggagaagtcgatcaaggagacgctcagcctgctgttcgacgacctcaaggcccagaagctcgacctgtccaagatatactttaagaacgacaagagcctcacggacctgtcccagcaggtattcgacgactacagcgtgatcgggacggccgtgctggagtacatcacccaacagatcgcgcccaagaacctggacaacccgtccaagaaggaacaagagctaatcgccaaaaagactgagaaggcgaagtacctgtcgctggagacgatcaagctcgcgcttgaggagtttaacaagcaccgcgacatcgacaagcagtgccggttcgaggagatcctggccaacttcgcggcgatcccgatgatcttcgacgagatcgcccagaacaaggacaacctggcgcagatcagcatcaagtaccagaaccagggcaaaaaagacttgctccaagctagtgcggaggacgacgtgaaggcgattaaggatctgctggaccagactaacaatctgctgcacaagctcaagatctttcacatctctcagtcggaggacaaggcgaacatcctggacaaggacgagcacttctacctagtgttcgaggagtgctacttcgagctggcgaacatcgtgcccctgtacaacaagatccggaactacatcacccagaagccctacagcgacgagaagttcaagctgaacttcgagaacagcacgctggcgaacgggtgggacaagaacaaggagcccgacaacaccgccatcctgttcatcaaggacgacaaatattacctcggcgtcatgaacaaaaagaataacaagatattcgatgacaaggcgatcaaggagaacaagggcgagggctacaagaagatcgtatataaactcctgccgggagcgaacaagatgctcccgaaggttttctttagtgccaagtccatcaagttctacaaccccagcgaggacatcctccgcatccggaaccactccacccacaccaagaacggctcgccgcagaagggctacgagaagttcgagttcaacatcgaggactgccgcaagttcatcgacttctacaagcagtccatctccaagcacccggagtggaaggacttcgggttccggttctccgacacgcagcgctacaactccatcgacgagttctaccgggaggtggagaaccagggctacaagctgacgttcgagaacatctcggagtcctacatcgactccgtggtcaaccagggcaagctgtacctcttccagatatacaataaggacttctccgcctacagcaaggggcggcccaacctccacaccctgtactggaaggcgctcttcgacgagcggaacctccaggacgtcgtgtacaagctgaacggcgaggcggagctgttctaccgcaagcagagcatccccaagaagatcacgcaccccgcgaaggaggccatcgccaacaagaacaaggacaaccccaagaaggaatcggtcttcgagtacgacctcatcaaggacaagcggttcacggaggacaaattctttttccactgcccgatcactattaacttcaagtccagcggcgcgaacaagttcaacgacgagatcaacctgctcctcaaggagaaggcgaacgacgtgcacatcctcagcatcgaccggggcgagcgccacctcgcctactacacgctggtggacgggaagggcaacatcatcaagcaagacaccttcaacatcatcggcaacgaccggatgaagaccaactaccacgacaagctggccgccatcgagaaggaccgcgactcggcccgcaaggactggaaaaagatcaacaatatcaaggagatgaaggagggctacctgagccaagttgtccacgagatcgccaagctggtgatcgagtacaacgccatcgtcgtgttcgaagacctgaacttcggcttcaagcgcggccggttcaaggtcgagaaacaagtctatcagaaacttgagaagatgctgatcgagaagctgaactacctcgtcttcaaggacaacgagttcgacaagaccggcggcgtcctccgcgcgtaccagctcaccgcgccgttcgagacgttcaagaaaatgggcaagcagaccggcatcatctactacgtgcccgccgggttcacgagcaaaatatgtcccgtgaccggcttcgtcaaccagctctaccccaagtacgagtccgtgtcgaagtcccaggaattcttcagcaagttcgacaagatatgctacaacctggacaagggctacttcgagttctccttcgactacaagaacttcggggacaaggcggcgaaggggaagtggaccatcgcctcgttcgggtcgcgcctcatcaacttccggaacagcgacaagaaccacaactgggacacccgcgaggtgtacccgacgaaggagctggagaagctcctcaaggactacagcatcgagtacggccacggggagtgcatcaaggcggccatctgcggcgagtcggacaaaaagttctttgccaaactcacctcggtcctcaacaccatcctccagatgcggaacagcaagacgggcacggagctggactacctcatcagcccggtggccgacgtgaacggcaatttctttgactcacgccaggcccctaagaacatgccccaggacgccgacgccaacggcgcgtaccacatcggcctcaagggcctgatgctgctcggccggatcaagaacaaccaggagggcaagaagctcaacctggtcatcaagaacgaggagtatttcgagttcgtccagaaccgcaacaacggatctaagaagcgtaggatcaagcaagattagttaattaagggcccatccaagtaaattaagttggatcagtagagatgcatggttggtgttctcatgtggtctgaatctgtgttctgtttcatcttttgtgtaagtgtgcaacttcgattctgtaaaacttggcaagtagatgaacgatgtcatttatatgcgtgtagcttgtcaaaatacaaagtagaaacctgtatctgaataagatggcattcatgaaataatattatcgattgaacgaacgaagcaacagaatgatcttccaaattgaagcaaaggaattgatggacaacgagttgctgtactttggagctgtaaatctgtagtacttctcaacgatgctatggaccgtgggccgtccataatcgctgggtggaatgagcccactgtccatgtgcttctgcatttggcccgtgtacagcccatataatgtcgaaaaacctaatacgcgcgcgccgccggcgcgcgtttcggacccagaacgcctagcgccgctgcgccgacgtgcgcgcgtctccactttttttttcgttttctttccatt

SEQ ID NO: 68 - DNA, 3960 bp, synthetic construct
  misc_feature 1..3960: note = Synthetic polynucleotide.NLS-FnCpf1-CO5-NLS
  source 1..3960: mol_type = other DNA
ggtagcaaaaagaggcgtatcaagcaggacatgtccatataccaggagtttgttaataaatatagcttgtccaaaaccctgcggtttgaactcatacctcaggggaagactttggagaatatcaaagcgcggggactgatactggacgacgaaaagcgcgcaaaagattacaagaaagcgaaacagatcatcgataaataccatcaatttttcatagaggagattctcagttctgtctgtatcagtgaggacctcctccaaaattattcagacgtctattttaaactcaagaagtcggacgacgacaaccttcagaaagattttaagtcagcaaaagacacaatcaaaaaacaaatatcggaatacataaaggactcagaaaagttcaagaatctttttaaccaaaatctgatagacgcgaagaaagggcaggaatctgatcttatactctggcttaagcagtctaaagacaacggcatagaactctttaaggcaaacagcgatataaccgacatagatgaagccctcgagataattaagtccttcaaaggctggactacatattttaaagggttccatgagaataggaagaacgtgtattcctcgaatgatattcccacctcgataatctaccggattgtggatgataatctgcctaaatttttggaaaataaagcgaagtacgaaagtttgaaagataaagcaccagaagcaattaattatgaacaaattaagaaagatctggctgaggaacttacgttcgatatcgattataaaacatcagaagttaatcagcgggtttttagcctggatgaagtttttgagatcgccaacttcaacaattatcttaatcagagcgggattaccaaattcaacactattatcggtggtaaattcgttaatggtgagaacacaaagagaaaaggtataaacgaatacataaatttgtacagtcaacagattaacgataaaactttgaaaaagtacaagatgtcagtgcttttcaaacaaatcctttccgacacagaatccaaaagttttgtgatagacaaattggaagatgatagcgacgtcgtcacgaccatgcaatcattttatgagcaaattgcagccttcaagacggttgaggaaaaaagtataaaagaaacgttgtcgctcttgttcgacgacctgaaagcacagaaattggatttgtctaagatatactttaaaaacgacaaatccctcacggacttgtcgcagcaagtctttgatgattattcggtgattgggacagccgtgctcgaatacatcacccagcaaatcgctccgaaaaatctggacaatccgtctaaaaaagagcaagagctgatcgcaaagaaaaccgaaaaagcgaaatacctgtctctggagacaatcaaattggccttggaagagttcaataagcacagagacattgataaacaatgtcggtttgaggaaattcttgctaactttgccgctatcccgatgatcttcgatgagattgcgcaaaataaggataatctggcccaaatctcgatcaagtatcaaaatcagggtaagaaggacctgcttcaagcatcggcggaggatgatgtgaaagcgattaaggacttgttggatcagaccaacaatttgttgcacaagctgaagatattccacatctcccagagtgaggataaggccaacatcctggacaaggatgaacatttctatttggtcttcgaagagtgttattttgaattggccaacatagttcctctttataacaagatccgcaattatattacacaaaagccttattccgatgaaaaatttaaacttaacttcgaaaatagcacattggcgaatggttgggataaaaataaggagcctgacaatactgctatacttttcattaaggacgataagtactacctcggcgttatgaacaagaagaataataagatctttgacgacaaagcaatcaaagagaacaaaggcgaaggttacaaaaaaatcgtgtacaaactcctgcctggcgcgaataaaatgcttccgaaggtttttttcagtgcgaagtccattaagttttataacccttccgaggatattttgagaattagaaatcactccacccataccaagaatggcagcccccagaaggggtatgaaaagttcgaatttaatatcgaagactgccgcaagtttatagacttttataaacagtccatatctaaacatcccgaatggaaagattttggtttccggttttctgacactcagaggtacaacagcatagatgagttctaccgcgaagttgaaaaccaaggctacaagcttacatttgagaacatcagcgagtcatatattgactcagtcgttaatcagggcaaactttatttgttccaaatttacaacaaagacttttcagcgtacagcaagggaaggccaaatctccatacactgtattggaaggcgctgtttgacgagcggaatcttcaagatgttgtgtataaactcaacggggaggccgaattgttctacaggaagcaaagcattcctaagaaaattacccaccccgctaaggaagcgatagcaaataaaaataaagacaatccgaaaaaagagagcgtttttgagtacgaccttataaaggataagagattcaccgaggataagtttttcttccactgtccaataactattaactttaaatcctccggagccaacaagtttaacgacgaaattaatttgctgctgaaagagaaggcgaacgacgttcacattctgtcaatagacagaggggagagacacttggcatactacacgctcgttgatggaaaaggtaacattattaagcaagatactttcaacatcattgggaatgacagaatgaaaacaaactatcacgataaactcgcggcaattgagaaggaccgggattcggcgaggaaagactggaagaaaatcaacaatataaaggagatgaaggaaggatacctgtctcaagtcgtccacgaaatagccaagcttgttatagagtataatgcgattgtggtctttgaagaccttaactttggatttaaacgcggccggtttaaagtcgagaaacaagtgtatcaaaaactggaaaaaatgttgatcgagaaactgaattatctcgtcttcaaggacaacgagttcgataaaaccgggggcgtcttgcgcgcttatcaactgacggcaccgtttgaaactttcaagaagatgggcaaacagactgggattatctactacgttcctgccggattcacctccaaaatatgcccagttactggatttgttaaccagttgtaccctaagtacgaatcagtcagcaagtcccaagagtttttctcaaaatttgataagatctgctataacctggacaaggggtacttcgagttttccttcgactataaaaatttcggcgacaaggcagctaaaggtaaatggacgattgcaagtttcggctccaggcttattaatttcagaaacagcgacaaaaaccataactgggacacgcgcgaggtctaccctacgaaggaactggaaaaacttctcaaggattacagtatagaatacggacacggggagtgtatcaaagctgcgatatgcggagagtcggataaaaagtttttcgcaaagttgacatcagttttgaacactatcttgcagatgagaaattccaagactggcacggagttggactaccttattagcccagtggcggatgtcaacgggaacttttttgattcgaggcaagcccctaagaatatgcctcaagacgctgatgcaaacggggcatatcacataggactcaaagggttgatgctgctgggcagaattaagaacaaccaggaaggaaagaagctcaatcttgttatcaaaaatgaagagtactttgaattcgttcaaaaccggaataacggatctaagaagcgtaggatcaagcaagat

SEQ ID NO: 69 - DNA, 6530 bp, synthetic construct
  misc_feature 1..6530: note = Synthetic polynucleotide.Expression cassette comprising Zea maysUbiquitin promoter cassette, NLS-FnCpf1-CO5-NLS and an Oryzasativa transcription termination sequence
  source 1..6530: mol_type = other DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgttgggtgatacttctgcagcggaccgacggtaccatgggtagcaaaaagaggcgtatcaagcaggacatgtccatataccaggagtttgttaataaatatagcttgtccaaaaccctgcggtttgaactcatacctcaggggaagactttggagaatatcaaagcgcggggactgatactggacgacgaaaagcgcgcaaaagattacaagaaagcgaaacagatcatcgataaataccatcaatttttcatagaggagattctcagttctgtctgtatcagtgaggacctcctccaaaattattcagacgtctattttaaactcaagaagtcggacgacgacaaccttcagaaagattttaagtcagcaaaagacacaatcaaaaaacaaatatcggaatacataaaggactcagaaaagttcaagaatctttttaaccaaaatctgatagacgcgaagaaagggcaggaatctgatcttatactctggcttaagcagtctaaagacaacggcatagaactctttaaggcaaacagcgatataaccgacatagatgaagccctcgagataattaagtccttcaaaggctggactacatattttaaagggttccatgagaataggaagaacgtgtattcctcgaatgatattcccacctcgataatctaccggattgtggatgataatctgcctaaatttttggaaaataaagcgaagtacgaaagtttgaaagataaagcaccagaagcaattaattatgaacaaattaagaaagatctggctgaggaacttacgttcgatatcgattataaaacatcagaagttaatcagcgggtttttagcctggatgaagtttttgagatcgccaacttcaacaattatcttaatcagagcgggattaccaaattcaacactattatcggtggtaaattcgttaatggtgagaacacaaagagaaaaggtataaacgaatacataaatttgtacagtcaacagattaacgataaaactttgaaaaagtacaagatgtcagtgcttttcaaacaaatcctttccgacacagaatccaaaagttttgtgatagacaaattggaagatgatagcgacgtcgtcacgaccatgcaatcattttatgagcaaattgcagccttcaagacggttgaggaaaaaagtataaaagaaacgttgtcgctcttgttcgacgacctgaaagcacagaaattggatttgtctaagatatactttaaaaacgacaaatccctcacggacttgtcgcagcaagtctttgatgattattcggtgattgggacagccgtgctcgaatacatcacccagcaaatcgctccgaaaaatctggacaatccgtctaaaaaagagcaagagctgatcgcaaagaaaaccgaaaaagcgaaatacctgtctctggagacaatcaaattggccttggaagagttcaataagcacagagacattgataaacaatgtcggtttgaggaaattcttgctaactttgccgctatcccgatgatcttcgatgagattgcgcaaaataaggataatctggcccaaatctcgatcaagtatcaaaatcagggtaagaaggacctgcttcaagcatcggcggaggatgatgtgaaagcgattaaggacttgttggatcagaccaacaatttgttgcacaagctgaagatattccacatctcccagagtgaggataaggccaacatcctggacaaggatgaacatttctatttggtcttcgaagagtgttattttgaattggccaacatagttcctctttataacaagatccgcaattatattacacaaaagccttattccgatgaaaaatttaaacttaacttcgaaaatagcacattggcgaatggttgggataaaaataaggagcctgacaatactgctatacttttcattaaggacgataagtactacctcggcgttatgaacaagaagaataataagatctttgacgacaaagcaatcaaagagaacaaaggcgaaggttacaaaaaaatcgtgtacaaactcctgcctggcgcgaataaaatgcttccgaaggtttttttcagtgcgaagtccattaagttttataacccttccgaggatattttgagaattagaaatcactccacccataccaagaatggcagcccccagaaggggtatgaaaagttcgaatttaatatcgaagactgccgcaagtttatagacttttataaacagtccatatctaaacatcccgaatggaaagattttggtttccggttttctgacactcagaggtacaacagcatagatgagttctaccgcgaagttgaaaaccaaggctacaagcttacatttgagaacatcagcgagtcatatattgactcagtcgttaatcagggcaaactttatttgttccaaatttacaacaaagacttttcagcgtacagcaagggaaggccaaatctccatacactgtattggaaggcgctgtttgacgagcggaatcttcaagatgttgtgtataaactcaacggggaggccgaattgttctacaggaagcaaagcattcctaagaaaattacccaccccgctaaggaagcgatagcaaataaaaataaagacaatccgaaaaaagagagcgtttttgagtacgaccttataaaggataagagattcaccgaggataagtttttcttccactgtccaataactattaactttaaatcctccggagccaacaagtttaacgacgaaattaatttgctgctgaaagagaaggcgaacgacgttcacattctgtcaatagacagaggggagagacacttggcatactacacgctcgttgatggaaaaggtaacattattaagcaagatactttcaacatcattgggaatgacagaatgaaaacaaactatcacgataaactcgcggcaattgagaaggaccgggattcggcgaggaaagactggaagaaaatcaacaatataaaggagatgaaggaaggatacctgtctcaagtcgtccacgaaatagccaagcttgttatagagtataatgcgattgtggtctttgaagaccttaactttggatttaaacgcggccggtttaaagtcgagaaacaagtgtatcaaaaactggaaaaaatgttgatcgagaaactgaattatctcgtcttcaaggacaacgagttcgataaaaccgggggcgtcttgcgcgcttatcaactgacggcaccgtttgaaactttcaagaagatgggcaaacagactgggattatctactacgttcctgccggattcacctccaaaatatgcccagttactggatttgttaaccagttgtaccctaagtacgaatcagtcagcaagtcccaagagtttttctcaaaatttgataagatctgctataacctggacaaggggtacttcgagttttccttcgactataaaaatttcggcgacaaggcagctaaaggtaaatggacgattgcaagtttcggctccaggcttattaatttcagaaacagcgacaaaaaccataactgggacacgcgcgaggtctaccctacgaaggaactggaaaaacttctcaaggattacagtatagaatacggacacggggagtgtatcaaagctgcgatatgcggagagtcggataaaaagtttttcgcaaagttgacatcagttttgaacactatcttgcagatgagaaattccaagactggcacggagttggactaccttattagcccagtggcggatgtcaacgggaacttttttgattcgaggcaagcccctaagaatatgcctcaagacgctgatgcaaacggggcatatcacataggactcaaagggttgatgctgctgggcagaattaagaacaaccaggaaggaaagaagctcaatcttgttatcaaaaatgaagagtactttgaattcgttcaaaaccggaataacggatctaagaagcgtaggatcaagcaagattgattaattaagggcccatccaagtaaattaagttggatcagtagagatgcatggttggtgttctcatgtggtctgaatctgtgttctgtttcatcttttgtgtaagtgtgcaacttcgattctgtaaaacttggcaagtagatgaacgatgtcatttatatgcgtgtagcttgtcaaaatacaaagtagaaacctgtatctgaataagatggcattcatgaaataatattatcgattgaacgaacgaagcaacagaatgatcttccaaattgaagcaaaggaattgatggacaacgagttgctgtactttggagctgtaaatctgtagtacttctcaacgatgctatggaccgtgggccgtccataatcgctgggtggaatgagcccactgtccatgtgcttctgcatttggcccgtgtacagcccatataatgtcgaaaaacctaatacgcgcgcgccgccggcgcgcgtttcggacccagaacgcctagcgccgctgcgccgacgtgcgcgcgtctccactttttttttcgttttctttccatt

SEQ ID NO: 70 - DNA, 3960 bp, synthetic construct
  misc_feature 1..3960: note = Synthetic polynucleotide.NLS-FnCpf1-CO6-NLS
  source 1..3960: mol_type = other DNA
ggtagcaaaaagaggcgtatcaagcaggacatgagtatttatcaagagttcgttaataagtacagtctttctaagacgctcaggtttgagctcattccacaaggtaagacccttgagaatattaaagcccgcggactgatcctggacgacgagaagcgcgccaaagactataagaaggcgaaacagatcatagataagtatcaccaattcttcatagaagagatacttagctctgtttgtatctcagaggatctgctccagaactactcggatgtgtactttaaactcaagaagtctgacgatgacaatctccagaaggactttaagtccgccaaagacacaatcaagaagcagatttcggaatacataaaggacagtgagaagttcaagaacttgttcaaccagaatctcatagacgccaagaagggtcaggagagcgatcttattctgtggttgaaacaatcaaaggacaacggtattgagttgtttaaagcaaactctgacatcaccgacattgacgaggctctggagatcattaagagttttaaagggtggacaacctactttaagggatttcatgagaatcgcaagaacgtgtactcgagcaatgacatacctacaagcataatctatcggatagtcgacgataacctcccgaagttcctcgagaataaagcgaagtacgaatcgcttaaggataaggcgccggaggcgataaactatgaacagattaagaaggatctggctgaagaattgacctttgatattgactataagaccagcgaagtcaaccaacgcgttttcagcctggatgaagtgtttgagatcgccaacttcaataattacttgaatcaatcggggataacaaagtttaatacgattatcggcgggaagtttgtcaacggggagaacacaaagaggaagggcataaatgagtacattaatctctatagtcaacagataaatgacaagacgttgaagaaatacaagatgtcagtccttttcaagcagatcctgtctgacacagaatcgaagagcttcgtgatagataagctggaagatgattccgacgttgtcacaaccatgcaatcgttttacgagcagatcgcggccttcaagaccgttgaggagaagtctattaaagagactctttctcttctgtttgatgacttgaaggcccagaagcttgacttgtccaagatctacttcaagaacgataaatctctgacagatctcagccagcaagtgtttgacgactattctgttatcggaacggccgtgctggagtacatcacacagcagatcgcgcctaagaaccttgataatccgagcaagaaagaacaggagttgattgcaaagaagacagagaaggccaaatacctttctctggagacaattaaacttgcactggaagaattcaataagcacagagatatcgacaagcagtgtcgctttgaggagatcctggcaaattttgctgccatcccaatgatttttgatgagattgcccagaacaaggacaatctcgcgcagatatcaatcaagtatcagaaccaaggcaagaaggacctcctgcaagcctcagccgaagatgacgttaaggccataaaggatctccttgatcagaccaataacttgctgcacaagctgaagatctttcatatcagccagtcggaagacaaagcaaatatccttgacaaggacgagcatttctatctggtgttcgaagaatgctatttcgagctggccaatatcgtgcctctgtacaataagatccgcaattatatcacgcagaagccgtatagcgacgagaagttcaagctgaatttcgagaactccaccttggccaatggttgggataagaacaaagaaccggacaatacggccatcttgtttatcaaagatgacaagtactatctgggagtgatgaataagaagaacaataagatcttcgatgacaaagccattaaggagaataaaggggaaggttacaagaagattgtctataaactgctccccggggccaacaagatgctgcccaaagtttttttcagtgccaagagcatcaagttttataatccctcagaagacatactgagaatcagaaaccactcgacccataccaagaacggctctccgcagaaggggtacgagaaattcgaattcaacatcgaagactgtagaaagttcatcgatttttacaagcaatcgatatcaaagcaccctgaatggaaggattttggttttcgctttagtgacactcagcggtataatagcattgatgagttctaccgcgaagttgagaatcagggatacaaattgacattcgagaatatcagcgaatcgtacattgatagcgtcgtcaaccaggggaaactttacctcttccagatttataataaagacttctcggcgtactccaagggaagaccaaatcttcatactctgtattggaaggcactcttcgatgagagaaatcttcaggatgtcgtttataaacttaatggggaagcggagctgttctaccgcaagcagagcatccctaagaagatcacgcaccccgcgaaggaggcgatagccaataagaacaaggataacccgaagaaggagtccgtcttcgaatatgacctgatcaaggataagagattcactgaggacaaattcttcttccattgccctattacgattaattttaaatcgagcggcgcgaataaattcaacgacgagatcaacctgctcctcaaagagaaggccaatgatgtccacattctctcaatcgacagaggggagaggcaccttgcctactatacgctcgttgatggtaaaggtaacatcattaagcaggacacgttcaacatcatcgggaacgaccgcatgaagacaaactatcacgataaattggcggcaatcgagaaggatcgggatagtgccaggaaggactggaagaagattaataacatcaaggagatgaaggagggatatctttctcaagtcgtccacgagatcgcgaagctggttatcgagtacaacgccatagtggtcttcgaagatctgaacttcggattcaagagagggagatttaaggtggagaagcaagtttatcagaaactggagaagatgctgatagagaagctcaactacctcgtgtttaaagacaacgagtttgacaagacaggtggggtgttgagggcctatcagctcacggcccccttcgagaccttcaagaagatgggaaagcagacggggataatctactacgtccctgctggcttcacttcgaagatctgcccagttacaggattcgttaaccagttgtatcccaaatacgagtccgtctcaaagtcacaagaattcttttctaaattcgataagatctgctacaacctggataagggctacttcgagtttagctttgactataagaatttcggggacaaagcggcaaaggggaagtggacaatcgcaagttttggctcccggctgattaactttcggaattctgacaagaatcacaactgggatactagagaggtctatccaactaaagagttggagaagttgctcaaggactactccattgaatatggtcacggggaatgcattaaagcggccatctgcggagagtccgataagaagttctttgccaaacttacatcggtcttgaacaccatactgcagatgcggaacagcaagacgggaacggaactcgactaccttatctcacctgtggcggatgttaatggaaactttttcgattcgaggcaggcgcccaagaacatgccgcaagatgcagatgccaatggagcatatcacatcggtcttaaagggctcatgctgcttggccgcatcaagaacaaccaagaggggaagaagttgaacctggttattaagaacgaagaatacttcgaatttgtccagaaccgcaacaacggatctaagaagcgtaggatcaagcaagat

SEQ ID NO: 71 - DNA, 6530 bp, synthetic construct
  misc_feature 1..6530: note = Synthetic polynucleotide.Expression cassette comprising Zea maysUbiquitin promoter cassette, NLS-FnCpf1-CO6-NLS and an Oryzasativa transcription termination sequence
  source 1..6530: mol_type = other DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgttgggtgatacttctgcagcggaccgacggtaccatgggtagcaaaaagaggcgtatcaagcaggacatgagtatttatcaagagttcgttaataagtacagtctttctaagacgctcaggtttgagctcattccacaaggtaagacccttgagaatattaaagcccgcggactgatcctggacgacgagaagcgcgccaaagactataagaaggcgaaacagatcatagataagtatcaccaattcttcatagaagagatacttagctctgtttgtatctcagaggatctgctccagaactactcggatgtgtactttaaactcaagaagtctgacgatgacaatctccagaaggactttaagtccgccaaagacacaatcaagaagcagatttcggaatacataaaggacagtgagaagttcaagaacttgttcaaccagaatctcatagacgccaagaagggtcaggagagcgatcttattctgtggttgaaacaatcaaaggacaacggtattgagttgtttaaagcaaactctgacatcaccgacattgacgaggctctggagatcattaagagttttaaagggtggacaacctactttaagggatttcatgagaatcgcaagaacgtgtactcgagcaatgacatacctacaagcataatctatcggatagtcgacgataacctcccgaagttcctcgagaataaagcgaagtacgaatcgcttaaggataaggcgccggaggcgataaactatgaacagattaagaaggatctggctgaagaattgacctttgatattgactataagaccagcgaagtcaaccaacgcgttttcagcctggatgaagtgtttgagatcgccaacttcaataattacttgaatcaatcggggataacaaagtttaatacgattatcggcgggaagtttgtcaacggggagaacacaaagaggaagggcataaatgagtacattaatctctatagtcaacagataaatgacaagacgttgaagaaatacaagatgtcagtccttttcaagcagatcctgtctgacacagaatcgaagagcttcgtgatagataagctggaagatgattccgacgttgtcacaaccatgcaatcgttttacgagcagatcgcggccttcaagaccgttgaggagaagtctattaaagagactctttctcttctgtttgatgacttgaaggcccagaagcttgacttgtccaagatctacttcaagaacgataaatctctgacagatctcagccagcaagtgtttgacgactattctgttatcggaacggccgtgctggagtacatcacacagcagatcgcgcctaagaaccttgataatccgagcaagaaagaacaggagttgattgcaaagaagacagagaaggccaaatacctttctctggagacaattaaacttgcactggaagaattcaataagcacagagatatcgacaagcagtgtcgctttgaggagatcctggcaaattttgctgccatcccaatgatttttgatgagattgcccagaacaaggacaatctcgcgcagatatcaatcaagtatcagaaccaaggcaagaaggacctcctgcaagcctcagccgaagatgacgttaaggccataaaggatctccttgatcagaccaataacttgctgcacaagctgaagatctttcatatcagccagtcggaagacaaagcaaatatccttgacaaggacgagcatttctatctggtgttcgaagaatgctatttcgagctggccaatatcgtgcctctgtacaataagatccgcaattatatcacgcagaagccgtatagcgacgagaagttcaagctgaatttcgagaactccaccttggccaatggttgggataagaacaaagaaccggacaatacggccatcttgtttatcaaagatgacaagtactatctgggagtgatgaataagaagaacaataagatcttcgatgacaaagccattaaggagaataaaggggaaggttacaagaagattgtctataaactgctccccggggccaacaagatgctgcccaaagtttttttcagtgccaagagcatcaagttttataatccctcagaagacatactgagaatcagaaaccactcgacccataccaagaacggctctccgcagaaggggtacgagaaattcgaattcaacatcgaagactgtagaaagttcatcgatttttacaagcaatcgatatcaaagcaccctgaatggaaggattttggttttcgctttagtgacactcagcggtataatagcattgatgagttctaccgcgaagttgagaatcagggatacaaattgacattcgagaatatcagcgaatcgtacattgatagcgtcgtcaaccaggggaaactttacctcttccagatttataataaagacttctcggcgtactccaagggaagaccaaatcttcatactctgtattggaaggcactcttcgatgagagaaatcttcaggatgtcgtttataaacttaatggggaagcggagctgttctaccgcaagcagagcatccctaagaagatcacgcaccccgcgaaggaggcgatagccaataagaacaaggataacccgaagaaggagtccgtcttcgaatatgacctgatcaaggataagagattcactgaggacaaattcttcttccattgccctattacgattaattttaaatcgagcggcgcgaataaattcaacgacgagatcaacctgctcctcaaagagaaggccaatgatgtccacattctctcaatcgacagaggggagaggcaccttgcctactatacgctcgttgatggtaaaggtaacatcattaagcaggacacgttcaacatcatcgggaacgaccgcatgaagacaaactatcacgataaattggcggcaatcgagaaggatcgggatagtgccaggaaggactggaagaagattaataacatcaaggagatgaaggagggatatctttctcaagtcgtccacgagatcgcgaagctggttatcgagtacaacgccatagtggtcttcgaagatctgaacttcggattcaagagagggagatttaaggtggagaagcaagtttatcagaaactggagaagatgctgatagagaagctcaactacctcgtgtttaaagacaacgagtttgacaagacaggtggggtgttgagggcctatcagctcacggcccccttcgagaccttcaagaagatgggaaagcagacggggataatctactacgtccctgctggcttcacttcgaagatctgcccagttacaggattcgttaaccagttgtatcccaaatacgagtccgtctcaaagtcacaagaattcttttctaaattcgataagatctgctacaacctggataagggctacttcgagtttagctttgactataagaatttcggggacaaagcggcaaaggggaagtggacaatcgcaagttttggctcccggctgattaactttcggaattctgacaagaatcacaactgggatactagagaggtctatccaactaaagagttggagaagttgctcaaggactactccattgaatatggtcacggggaatgcattaaagcggccatctgcggagagtccgataagaagttctttgccaaacttacatcggtcttgaacaccatactgcagatgcggaacagcaagacgggaacggaactcgactaccttatctcacctgtggcggatgttaatggaaactttttcgattcgaggcaggcgcccaagaacatgccgcaagatgcagatgccaatggagcatatcacatcggtcttaaagggctcatgctgcttggccgcatcaagaacaaccaagaggggaagaagttgaacctggttattaagaacgaagaatacttcgaatttgtccagaaccgcaacaacggatctaagaagcgtaggatcaagcaagattgattaattaagggcccatccaagtaaattaagttggatcagtagagatgcatggttggtgttctcatgtggtctgaatctgtgttctgtttcatcttttgtgtaagtgtgcaacttcgattctgtaaaacttggcaagtagatgaacgatgtcatttatatgcgtgtagcttgtcaaaatacaaagtagaaacctgtatctgaataagatggcattcatgaaataatattatcgattgaacgaacgaagcaacagaatgatcttccaaattgaagcaaaggaattgatggacaacgagttgctgtactttggagctgtaaatctgtagtacttctcaacgatgctatggaccgtgggccgtccataatcgctgggtggaatgagcccactgtccatgtgcttctgcatttggcccgtgtacagcccatataatgtcgaaaaacctaatacgcgcgcgccgccggcgcgcgtttcggacccagaacgcctagcgccgctgcgccgacgtgcgcgcgtctccactttttttttcgttttctttccatt

SEQ ID NO: 72 - DNA, 3960 bp, synthetic construct
  misc_feature 1..3960: note = Synthetic polynucleotide.NLS-FnCpf1-CO7-NLS
  source 1..3960: mol_type = other DNA
ggtagcaaaaagaggcgtatcaagcaggacatgtccatctatcaggaatttgttaacaagtactctcttagcaaaactcttaggttcgaattgatacctcagggaaagacacttgagaatattaaggcgcgcgggctgatacttgatgatgaaaagcgggcaaaggactataagaaagctaagcaaataattgataagtaccaccagttttttattgaagagatcctgtcctccgtttgtatatccgaagacttgcttcagaattactcagatgtttattttaaattgaagaaatctgacgatgataatcttcaaaaggatttcaaatcggcaaaagacacaatcaaaaaacagattagcgagtacatcaaggactccgaaaagtttaagaatctctttaatcagaatcttatagacgcaaagaaaggacaggaatcggacttgattttgtggttgaagcagtccaaggataacgggatagaactctttaaagccaactccgatataacggacatcgacgaagccctcgaaatcattaagtcgtttaaaggttggaccacgtacttcaaaggattccacgagaacagaaagaacgtttactcgagcaacgacattcctactagcatcatatatagaatagtggatgataatttgcccaaattccttgagaacaaggcaaaatatgaatccctgaaagacaaggcgcccgaagctatcaactacgagcagataaagaaagatctggctgaggagctgacgtttgacattgattacaaaacaagcgaggtcaaccagagggttttttcgctggacgaggtttttgaaatagcgaattttaataactacctgaaccaatccggcatcactaaatttaacacaatcataggggggaagttcgtgaacggagagaacacaaagcgcaaagggattaatgagtacatcaatttgtacagccagcagatcaatgacaaaacgttgaagaaatataagatgagcgtcttgtttaagcagattctctcggatacggaatccaaatcattcgtcattgacaagctggaggatgacagcgatgtggtgacaactatgcagtccttctatgaacaaattgcagcttttaaaacggtcgaagagaagtcgattaaagaaacgctttcgctcctgttcgatgacctgaaagcgcagaagctggacctttcgaagatatatttcaaaaacgataagtcactcacggaccttagccaacaggttttcgatgactattctgtcatagggactgctgtgcttgagtatattactcagcaaatagcccccaaaaacctggacaacccgtctaagaaggaacaagagctgatagcgaagaaaacagagaaagctaaatatctttcacttgaaactataaagcttgcactggaggaattcaacaaacatcgcgacatcgacaagcagtgcagatttgaggagatcttggccaacttcgcagctattccaatgatttttgacgagatagcacagaataaggacaacctggcacagattagcataaaataccagaatcaggggaagaaggatcttcttcaggcttcggctgaggacgatgtcaaagccatcaaagatctgctcgaccagaccaacaatttgttgcataaactcaagatcttccacatatcgcagtccgaagataaagcgaacatacttgacaaagacgaacatttttatcttgttttcgaggagtgttattttgagttggcaaacatcgttcccctctacaacaaaattcgcaactatataactcagaagccctattctgacgagaaattcaaacttaatttcgaaaacagcactctcgcaaacggctgggataaaaacaaggaacccgacaacaccgccatactttttattaaggatgataaatattatttgggcgtgatgaataaaaagaacaataaaatattcgatgataaagcaattaaggaaaataaaggggaagggtataaaaaaatcgtgtataagctgcttccaggagctaataaaatgctgccaaaagtcttcttctctgccaagtcgatcaagttttacaatccttctgaagatattttgcggatcagaaatcactctactcacactaaaaacggttcaccccagaaaggatacgagaagtttgagttcaacatcgaagactgtcggaagtttatcgacttttacaagcagtctatatcaaaacaccccgaatggaaagattttggttttcggttcagcgacacgcagagatataattcaattgatgagttctacagggaggtggagaaccaagggtataaacttacttttgagaacatttccgaatcctatattgattcggtggtcaatcaggggaaactgtacctgtttcagatatataacaaagacttctccgcgtattctaaaggacggcccaatctccatactctttattggaaggcgctgtttgacgagcggaaccttcaggatgttgtctataagttgaacggggaagctgagctgttctatcggaagcagtctattccaaaaaagataacgcaccccgcgaaggaggcaattgcaaacaagaacaaagacaatccaaagaaggagtcggtgtttgagtacgatctgataaaagacaaaaggtttaccgaggacaagttttttttccactgtccgatcaccatcaattttaagtcttccggcgccaacaaattcaatgacgaaataaatctgctgctcaaggaaaaggcaaatgatgttcatattctgtcgatagaccgcggggaaagacacctcgcgtattatacattggtcgatgggaaaggcaatattatcaaacaagacaccttcaatatcattggtaacgataggatgaaaacgaactatcatgataaacttgcagctattgaaaaggacagagactcggctcggaaagattggaaaaagatcaataacatcaaggaaatgaaggaagggtatctctcccaggtcgttcatgaaatcgccaaactggttattgagtacaatgctatagtggtttttgaagatcttaattttgggtttaagagagggagatttaaagtcgagaaacaggtttatcaaaaacttgaaaaaatgttgatagaaaaattgaactatcttgtgtttaaggacaatgagttcgataaaaccgggggggttttgagagcttatcaactgacggctcccttcgagacattcaaaaaaatggggaagcagaccggcatcatttattatgtgcccgcgggcttcacttctaaaatttgtcctgtgacagggttcgtgaaccaattgtaccccaagtacgaaagcgtctccaagtcccaagaattctttagcaagtttgacaaaatttgctataacctggacaaagggtactttgagttttcctttgattataagaatttcggggataaagctgcgaaaggtaagtggacgatagcctctttcgggtcgcgccttattaacttcaggaattccgacaaaaaccataattgggacacccgcgaggtctaccctacaaaggaactcgaaaagttgcttaaggattattcaatagagtatggtcatggcgagtgtattaaggctgctatctgtggtgagtcagataagaaattcttcgcgaaattgacatctgttttgaacaccatcctccaaatgaggaactctaaaacggggacggagcttgattatctcatctcacccgttgctgatgtgaacggtaatttctttgattcacgccaagccccgaagaacatgccccaagacgccgatgctaacggcgcttatcatatagggctgaaggggctcatgcttctggggcgcatcaaaaataaccaagaagggaagaaactcaatctggttatcaaaaatgaggaatatttcgaattcgtgcaaaaccgcaacaatggatctaagaagcgtaggatcaagcaagat

SEQ ID NO: 73 - DNA, 6530 bp, synthetic construct
  misc_feature 1..6530: note = Synthetic polynucleotide.Expression cassette comprising Zea maysUbiquitin promoter cassette, NLS-FnCpf1-CO7-NLS and an Oryzasativa transcription termination sequence
  source 1..6530: mol_type = other DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgttgggtgatacttctgcagcggaccgacggtaccatgggtagcaaaaagaggcgtatcaagcaggacatgtccatctatcaggaatttgttaacaagtactctcttagcaaaactcttaggttcgaattgatacctcagggaaagacacttgagaatattaaggcgcgcgggctgatacttgatgatgaaaagcgggcaaaggactataagaaagctaagcaaataattgataagtaccaccagttttttattgaagagatcctgtcctccgtttgtatatccgaagacttgcttcagaattactcagatgtttattttaaattgaagaaatctgacgatgataatcttcaaaaggatttcaaatcggcaaaagacacaatcaaaaaacagattagcgagtacatcaaggactccgaaaagtttaagaatctctttaatcagaatcttatagacgcaaagaaaggacaggaatcggacttgattttgtggttgaagcagtccaaggataacgggatagaactctttaaagccaactccgatataacggacatcgacgaagccctcgaaatcattaagtcgtttaaaggttggaccacgtacttcaaaggattccacgagaacagaaagaacgtttactcgagcaacgacattcctactagcatcatatatagaatagtggatgataatttgcccaaattccttgagaacaaggcaaaatatgaatccctgaaagacaaggcgcccgaagctatcaactacgagcagataaagaaagatctggctgaggagctgacgtttgacattgattacaaaacaagcgaggtcaaccagagggttttttcgctggacgaggtttttgaaatagcgaattttaataactacctgaaccaatccggcatcactaaatttaacacaatcataggggggaagttcgtgaacggagagaacacaaagcgcaaagggattaatgagtacatcaatttgtacagccagcagatcaatgacaaaacgttgaagaaatataagatgagcgtcttgtttaagcagattctctcggatacggaatccaaatcattcgtcattgacaagctggaggatgacagcgatgtggtgacaactatgcagtccttctatgaacaaattgcagcttttaaaacggtcgaagagaagtcgattaaagaaacgctttcgctcctgttcgatgacctgaaagcgcagaagctggacctttcgaagatatatttcaaaaacgataagtcactcacggaccttagccaacaggttttcgatgactattctgtcatagggactgctgtgcttgagtatattactcagcaaatagcccccaaaaacctggacaacccgtctaagaaggaacaagagctgatagcgaagaaaacagagaaagctaaatatctttcacttgaaactataaagcttgcactggaggaattcaacaaacatcgcgacatcgacaagcagtgcagatttgaggagatcttggccaacttcgcagctattccaatgatttttgacgagatagcacagaataaggacaacctggcacagattagcataaaataccagaatcaggggaagaaggatcttcttcaggcttcggctgaggacgatgtcaaagccatcaaagatctgctcgaccagaccaacaatttgttgcataaactcaagatcttccacatatcgcagtccgaagataaagcgaacatacttgacaaagacgaacatttttatcttgttttcgaggagtgttattttgagttggcaaacatcgttcccctctacaacaaaattcgcaactatataactcagaagccctattctgacgagaaattcaaacttaatttcgaaaacagcactctcgcaaacggctgggataaaaacaaggaacccgacaacaccgccatactttttattaaggatgataaatattatttgggcgtgatgaataaaaagaacaataaaatattcgatgataaagcaattaaggaaaataaaggggaagggtataaaaaaatcgtgtataagctgcttccaggagctaataaaatgctgccaaaagtcttcttctctgccaagtcgatcaagttttacaatccttctgaagatattttgcggatcagaaatcactctactcacactaaaaacggttcaccccagaaaggatacgagaagtttgagttcaacatcgaagactgtcggaagtttatcgacttttacaagcagtctatatcaaaacaccccgaatggaaagattttggttttcggttcagcgacacgcagagatataattcaattgatgagttctacagggaggtggagaaccaagggtataaacttacttttgagaacatttccgaatcctatattgattcggtggtcaatcaggggaaactgtacctgtttcagatatataacaaagacttctccgcgtattctaaaggacggcccaatctccatactctttattggaaggcgctgtttgacgagcggaaccttcaggatgttgtctataagttgaacggggaagctgagctgttctatcggaagcagtctattccaaaaaagataacgcaccccgcgaaggaggcaattgcaaacaagaacaaagacaatccaaagaaggagtcggtgtttgagtacgatctgataaaagacaaaaggtttaccgaggacaagttttttttccactgtccgatcaccatcaattttaagtcttccggcgccaacaaattcaatgacgaaataaatctgctgctcaaggaaaaggcaaatgatgttcatattctgtcgatagaccgcggggaaagacacctcgcgtattatacattggtcgatgggaaaggcaatattatcaaacaagacaccttcaatatcattggtaacgataggatgaaaacgaactatcatgataaacttgcagctattgaaaaggacagagactcggctcggaaagattggaaaaagatcaataacatcaaggaaatgaaggaagggtatctctcccaggtcgttcatgaaatcgccaaactggttattgagtacaatgctatagtggtttttgaagatcttaattttgggtttaagagagggagatttaaagtcgagaaacaggtttatcaaaaacttgaaaaaatgttgatagaaaaattgaactatcttgtgtttaaggacaatgagttcgataaaaccgggggggttttgagagcttatcaactgacggctcccttcgagacattcaaaaaaatggggaagcagaccggcatcatttattatgtgcccgcgggcttcacttctaaaatttgtcctgtgacagggttcgtgaaccaattgtaccccaagtacgaaagcgtctccaagtcccaagaattctttagcaagtttgacaaaatttgctataacctggacaaagggtactttgagttttcctttgattataagaatttcggggataaagctgcgaaaggtaagtggacgatagcctctttcgggtcgcgccttattaacttcaggaattccgacaaaaaccataattgggacacccgcgaggtctaccctacaaaggaactcgaaaagttgcttaaggattattcaatagagtatggtcatggcgagtgtattaaggctgctatctgtggtgagtcagataagaaattcttcgcgaaattgacatctgttttgaacaccatcctccaaatgaggaactctaaaacggggacggagcttgattatctcatctcacccgttgctgatgtgaacggtaatttctttgattcacgccaagccccgaagaacatgccccaagacgccgatgctaacggcgcttatcatatagggctgaaggggctcatgcttctggggcgcatcaaaaataaccaagaagggaagaaactcaatctggttatcaaaaatgaggaatatttcgaattcgtgcaaaaccgcaacaatggatctaagaagcgtaggatcaagcaagattgattaattaagggcccatccaagtaaattaagttggatcagtagagatgcatggttggtgttctcatgtggtctgaatctgtgttctgtttcatcttttgtgtaagtgtgcaacttcgattctgtaaaacttggcaagtagatgaacgatgtcatttatatgcgtgtagcttgtcaaaatacaaagtagaaacctgtatctgaataagatggcattcatgaaataatattatcgattgaacgaacgaagcaacagaatgatcttccaaattgaagcaaaggaattgatggacaacgagttgctgtactttggagctgtaaatctgtagtacttctcaacgatgctatggaccgtgggccgtccataatcgctgggtggaatgagcccactgtccatgtgcttctgcatttggcccgtgtacagcccatataatgtcgaaaaacctaatacgcgcgcgccgccggcgcgcgtttcggacccagaacgcctagcgccgctgcgccgacgtgcgcgcgtctccactttttttttcgttttctttccatt

SEQ ID NO: 74 - DNA, 6062 bp, synthetic construct; identical to SEQ ID NO: 15
  misc_feature 1..6062: note = Synthetic polynucleotide.Expression cassette comprising Zea maysUbiquitin promoter cassette, NLS-LbCpf1-CO2-NLS and an Oryzasativa transcription termination sequence
  source 1..6062: mol_type = other DNA

SEQ ID NO: 75 - DNA, 3684 bp, synthetic construct
  misc_feature 1..3684: note = Synthetic polynucleotide.Codon optimized LbCpf1(TAT)- CO2
  source 1..3684: mol_type = other DNA
atgtcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgcgcgggtgggacaaggacgtggagaccgaccgccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcac

SEQ ID NO: 76 - protein, 1228 aa, synthetic construct
  REGION 1..1228: note = Synthetic polypeptide.LbCpf1(TAT)-CO2 comprisingG532R/K538V/Y542R substitutions
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDVETDRRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

(19) United States
(12) Patent Application Publication
(10) Pub. No.: US 2024/0200086 A1
(43) Pub. Date: Jun. 20, 2024

(54) COMPOSITIONS AND METHODS FOR GENOME EDITING IN PLANTA

(71) Applicant: Monsanto Technology LLC, St. Louis, MO (US)

(72) Inventors: Stanislaw FLASINSKI, Ballwin, MO (US); Elysia KRIEGER, Kirkwood, MO (US); Ervin NAGY, Lake Saint Louis, MO (US); Krishnakumar SRIDHARAN, Cary, NC (US); Xudong YE, Chesterfield, MO (US)

(73) Assignee: Monsanto Technology LLC, St. Louis, MO (US)

(21) Appl. No.: 18/501,949

(22) Filed: Nov. 3, 2023

Related U.S. Application Data
(63) Continuation of application No. 17/817,196, filed on Aug. 3, 2022, now Pat. No. 11,859,191.
Application No. 17/817,196 is a continuation of application No. 16/563,581, filed on Sep. 6, 2019, now Pat. No. 11,414,669.
(60) Provisional application No. 62/727,784, filed on Sep. 6, 2018.

(51) Int. Cl. C12N 15/82

(52) U.S. Cl. CPC C12N 15/8213; C12N 15/8205; C12N 2310/20; C12N 2800/80

(57) ABSTRACT

This disclosure is related to plant-optimized recombinant nucleic acids encoding Cpf1 and their use in planta. Also disclosed are compositions, expression cassettes, and plant cells comprising the recombinant nucleic acids as well as methods and kits for modifying a target sequence in a plant genome using the recombinant nucleic acids.

### CROSS-REFERENCE TO RELATED APPLICATION

This application claims the benefit of U.S. Provisional Application No. 62/727,784, filed Sep. 6, 2018, which is incorporated by reference in its entirety herein.

### INCORPORATION OF SEQUENCE LISTING

A sequence listing contained in the file named P34668US01_SL.txt, which is 373,411 bytes (measured in MS-Windows®) and created on Sep. 5, 2019, and comprises 76 sequences, is filed electronically herewith and incorporated by reference in its entirety.

### FIELD

This disclosure relates to plant-optimized recombinant nucleic acids encoding Cpf1 and their use in planta.

### BACKGROUND

Clustered Regularly Interspaced Short Palindromic Repeats from Prevotella and Francisella 1 or CRISPR/Cpf1 (also known as Cas12a) was first demonstrated for genome editing in mammalian cells in 2015 (Zetsche et al., 2015, Cell 163, 759-771). Cpf1 (CRISPR from Prevotella and Francisella 1) is a large, 1,300 amino acid protein, belonging to class 2 CRISPR system. Different from Cas9 nuclease, the PAM motif of Cpf1 is located at 5′ of the target site and the mature gRNA is a single strand of approximately 44 bp.

Cpf1 genome editing in plants was first observed in rice (Xu et al., 2017, Plant Biotechnology Journal 15, 713-717), where up to 41% mutation rate was achieved at OsBel locus using pre-crRNA gRNA structure and LbCpf1. Subsequently, Cpf1 genome editing of rice and tobacco were observed in different laboratories using both LbCpf1 and FnCpf1 (Endo et al., Scientific Reports volume 6, Article number: 38169 (2016); Hu et al., 2017, Journal of Genetics and Genomics 44, 71-73; Tang et al., Nature Plants volume 3, Article number: 17018 (2017); Begemann et al., 2017, Sci Rep. 7, 11606). However, there remains a need for more effective Cpf1-based genome editing technologies in plants.

### SUMMARY

Several embodiments relate to a recombinant nucleic acid comprising a sequence selected from the group consisting of: SEQ ID NOs 1, 10, 38, 45-51 and 75. In some embodiments, the recombinant nucleic acid further comprises a nucleic acid sequence encoding one or more nuclear localization signals operably linked to the recombinant nucleic acid comprising a sequence selected from the group consisting of: SEQ ID NOs 1, 10, 38, 45-51 and 75. In some embodiments, the nuclear localization signal is provided on the 5′ end of Cpf1. In some embodiments, the nuclear localization signal is provided on the 3′ end of Cpf1. In some embodiments, the nuclear localization signal is provided on the 5′ and 3′ end of Cpf1. In some embodiments, the recombinant nucleic acid further comprises a promoter operably linked to the recombinant nucleic acid comprising a sequence selected from the group consisting of: SEQ ID NOs 1, 10, 38, 45-51 and 75. In some embodiments, the promoter comprises a sequence selected from the group consisting of SEQ ID NOs 7, 22, 27, and 32. In some embodiments, the recombinant nucleic acid further comprising one or more of an intron, a kozak sequence, a leader sequence and a terminator sequence. Several embodiments relate to a recombinant nucleic acid comprising a sequence selected from the group consisting of SEQ ID NOs 4, 6, 12, 14, 41, 63, 66, 68, 70, and 72.

Several embodiments relate to a plant cell comprising a recombinant nucleic acid comprising a sequence selected from the group consisting of: SEQ ID NOs 1, 10, 38, 45-51 and 75. Several embodiments relate to a plant cell comprising a recombinant nucleic acid comprising a nucleic acid sequence encoding one or more nuclear localization signals operably linked to the recombinant nucleic acid comprising a sequence selected from the group consisting of: SEQ ID NOs 1, 10, 38, 45-51 and 75. In some embodiments, the nuclear localization signal is provided on the 5′ end of Cpf1. In some embodiments, the nuclear localization signal is provided on the 3′ end of Cpf1. In some embodiments, the nuclear localization signal is provided on the 5′ and 3′ end of Cpf1. Several embodiments relate to a plant cell comprising a promoter operably linked to a recombinant nucleic acid comprising a sequence selected from the group consisting of: SEQ ID NOs 1, 10, 38, 45-51 and 75, and optionally one or more nuclear localization signals, an intron, a kozak sequence, a leader sequence and a terminator sequence. In some embodiments, the promoter comprises a sequence selected from the group consisting of SEQ ID NOs 7, 22, 27, and 32. Several embodiments relate to a plant cell comprising recombinant nucleic acid comprising a sequence selected from the group consisting of SEQ ID NOs 4, 6, 12, 14, 41, 63, 66, 68, 70, and 72. In some embodiments, the plant cell is a monocot or a dicot. In some embodiments, the plant cell is a maize, cotton, soybean, canola, wheat, tomato, rice, brassica, melon, cucurbit, or lettuce cell.

Several embodiments relate to an expression cassette comprising a recombinant nucleic acid sequence selected from the group consisting of SEQ ID NOs 15, 20, 26, 31, 36, 40, 56, 59, 65, 67, 69, 71, and 73. Several embodiments relate to a plant cell comprising an expression cassette comprising a recombinant nucleic acid sequence selected from the group consisting of SEQ ID NOs 15, 20, 26, 31, 36, 40, 56, 59, 65, 67, 69, 71, and 73. Several embodiments relate to an agrobacterium T-DNA vector comprising an expression SEQ ID NOs 15, 20, 26, 31, 36, 40, 56, 59, 65, 67, 69, 71, and 73. Several embodiments relate to an Agrobacterium cell comprising an agrobacterium T-DNA vector comprising an expression SEQ ID NOs 15, 20, 26, 31, 36, 40, 56, 59, 65, 67, 69, 71, and 73. In some embodiments, the agrobacterium T-DNA vector further comprises an expression cassette for a selectable marker gene. In some embodiments, the agrobacterium T-DNA vector further comprising a promoter operably linked to a one or more crRNA sequences and one or more spacer sequences, where in the spacer sequence is complementary to at least 23 base pairs of a target site. In some embodiments, the crRNA sequence is a pre-crRNA or a mature crRNA.

Several embodiments relate to a composition comprising: (a) recombinant nucleic acid comprising a sequence selected from the group consisting of: SEQ ID NOs 1, 10, 38, 45-51 and 75, and (b) a recombinant nucleic acid encoding a guide RNA comprised of at least one crRNA and at least one spacer RNA sequence. Several embodiments relate to a composition comprising: (a) recombinant nucleic acid comprising a sequence selected from the group consisting of: SEQ ID NOs 6, 12, 14, 41, 63, 66, 68, 70, and 72, and (b) a recombinant nucleic acid encoding a guide RNA comprised of at least one crRNA and at least one spacer RNA sequence. In some embodiments, the composition is provided on a particle suitable for biolistic delivery to a plant cell.

Several embodiments relate to a method for modifying a target sequence in the genome of a plant cell, comprising: introducing into the plant cell a recombinant nucleic acid comprising a sequence selected from the group consisting of: SEQ ID NOs 1, 10, 38, 45-51 and 75 operably linked to a promoter, and introducing into the plant cell a guide polynucleotide comprising a nucleic acid sequence that is complementary to a target sequence or a recombinant nucleic acid encoding the guide polynucleotide, wherein the guide polynucleotide and a Cpf1 nuclease expressed from the recombinant nucleic acid comprising a sequence selected from the group consisting of: SEQ ID NOs 1, 10, 38, 45-51 and 75 form a complex that can bind to and modify the target sequence. Several embodiments relate to a method for modifying a target sequence in the genome of a plant cell, comprising: introducing into the plant cell a recombinant nucleic acid comprising a sequence selected from the group consisting of: SEQ ID NOs 1, 10, 38, 45-51 and 75 operably linked to a promoter, and introducing into the plant cell a guide polynucleotide comprising a nucleic acid sequence that is complementary to a target sequence or a recombinant nucleic acid encoding the guide polynucleotide, wherein the guide polynucleotide and a Cpf1 nuclease expressed from the recombinant nucleic acid comprising a sequence selected from the group consisting of: SEQ ID NOs 1, 10, 38, 45-51 and 75 form a complex that can bind to and modify the target sequence, wherein the recombinant nucleic acid comprising a sequence selected from the group consisting of: SEQ ID NOs 1, 10, 38, 45-51 and 75 is operably linked to a promoter comprising a sequence selected from the group consisting of SEQ ID NOs 7, 22, 27, and 32. In some embodiments, the method further comprises incubating the plant cell at temperatures between 24° C. and 35° C. for a period of at least about 1-8 hours. In some embodiments, the method further comprises incubating the plant cell at temperatures between 28° C. and 35° C. for a period of at least about 4 hours. In some embodiments, the plant cell is a monocot or a dicot. In some embodiments, the plant cell is a maize, cotton, soybean, canola, wheat, tomato, rice, brassica, melon, cucurbit, or lettuce cell. In some embodiments, the method further comprises introducing a donor DNA to the plant cell. In some embodiments, the method further comprises identifying at least one plant cell comprising in its genome the donor DNA, or a portion thereof, integrated into or near said target sequence.

Several embodiments relate to a method for modifying a target sequence in the genome of a plant cell, the method comprising: introducing a guide polynucleotide comprising a nucleic acid sequence that is substantially complementary to the target sequence, or a recombinant nucleic acid encoding the guide polynucleotide, into a plant cell comprising in its genome a recombinant nucleic acid comprising a sequence selected from the group consisting of: SEQ ID NOs 1, 10, 38, 45-51 and 75 operably linked to a promoter, wherein the guide polynucleotide and a Cpf1 nuclease expressed from the recombinant nucleic acid comprising a sequence selected from the group consisting of: SEQ ID NOs 1, 10, 38, 45-51 and 75 form a complex that can bind to and modify the target sequence. Several embodiments relate to a method for modifying a target sequence in the genome of a plant cell, the method comprising: introducing a guide polynucleotide comprising a nucleic acid sequence that is substantially complementary to the target sequence, or a recombinant nucleic acid encoding the guide polynucleotide, into a plant cell comprising in its genome a recombinant nucleic acid comprising a sequence selected from the group consisting of: SEQ ID NOs 1, 10, 38, 45-51 and 75 operably linked to a promoter comprising a sequence selected from the group consisting of SEQ ID NOs 7, 22, 27, and 32, wherein the guide polynucleotide and a Cpf1 nuclease expressed from the recombinant nucleic acid comprising a sequence selected from the group consisting of: SEQ ID NOs 1, 10, 38, 45-51 and 75 form a complex that can bind to and modify the target sequence. In some embodiments, the method further comprises incubating the plant cell at temperatures between 24° C. and 35° C. for a period of at least about 1-8 hours. In some embodiments, the method further comprises incubating the plant cell at temperatures between 28° C. and 35° C. for a period of at least about 4 hours. In some embodiments, the plant cell is a monocot or a dicot. In some embodiments, the plant cell is a maize, cotton, soybean, canola, wheat, tomato, rice, brassica, melon, cucurbit, or lettuce cell. In some embodiments, the method further comprises introducing a donor DNA to the plant cell. In some embodiments, the method further comprises identifying at least one plant cell comprising in its genome the donor DNA, or a portion thereof, integrated into or near said target sequence.

Several embodiments relate to a kit for modifying a target sequence in the genome of a plant cell, the kit comprising a guide polynucleotide comprising a nucleic acid sequence that is complementary to a target sequence or a recombinant nucleic acid encoding the guide polynucleotide, and recombinant nucleic acid comprising a sequence selected from the group consisting of: SEQ ID NOs 1, 10, 38, 45-51 and 75. Several embodiments relate to a kit for modifying a target sequence in the genome of a plant cell, the kit comprising a guide polynucleotide comprising a nucleic acid sequence that is complementary to a target sequence or a recombinant nucleic acid encoding the guide polynucleotide, and recombinant nucleic acid comprising a sequence selected from the group consisting of: SEQ ID NOs 4, 6, 7, 12, 14, 15, 20, 22, 26, 27, 31, 32, 36, 40, 41, 56, 59, 63, 65, 66, 67, 68, 69, 70, 71, 72 and 73. Several embodiments relate to a kit for modifying a target sequence in the genome of a plant cell, the kit comprising a guide polynucleotide comprising a nucleic acid sequence that is complementary to a target sequence or a recombinant nucleic acid encoding the guide polynucleotide, a recombinant nucleic acid comprising a sequence selected from the group consisting of: SEQ ID NOs 1, 4, 6, 7, 10, 12, 14, 15, 20, 22, 26, 27, 31, 32, 36, 40, 41, 56, 59, 63, 65, 66, 67. 68, 69, 70, 71, 72, 73 and 75, and a recombinant nucleic acid encoding a selectable marker.

### BRIEF DESCRIPTION OF THE DRAWINGS

The accompanying drawings, which are incorporated in and form a part of the specification, illustrate the aspects of this disclosure and together with the description, serve to explain the principles of the disclosure. In the drawings:

FIG. 1 illustrates the expression of LbCpf1-mOrange fluorescent proteins in corn protoplasts denoted by average mOrange intensities.

FIG. 2 illustrates the expression of FnCpf1-mOrange fluorescent proteins in corn protoplasts denoted by average mOrange intensities.

### DETAILED DESCRIPTION

Unless defined otherwise, all technical and scientific terms used herein have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used.

The practice of the present disclosure employs, unless otherwise indicated, conventional techniques of biochemistry, chemistry, molecular biology, microbiology, cell biology, genomics, plant breeding, and biotechnology, which are within the skill of the art. See, e.g., Green and Sambrook, MOLECULAR CLONING: A LABORATORY MANUAL, 4th edition (2012); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)); Harlow and Lane, eds. (1988) ANTIBODIES, A LABORATORY MANUAL; ANIMAL CELL CULTURE (R. I. Freshney, ed. (1987)); RECOMBINANT PROTEIN PURIFICATION: PRINCIPLES AND METHODS, 18-1142-75, GE Healthcare Life Sciences; C. N. Stewart, A. Touracv, V. Citovsky, T. Tzfira eds. (2011) PLANT TRANSFORMATION TECHNOLOGIES (Wiley-Blackwell); and R. H. Smith (2013) PLANT TISSUE CULTURE. TECHNIQUES AND EXPERIMENTS (Academic Press, Inc.). The inventors do not intend to be limited to a mechanism or mode of action. Reference thereto is provided for illustrative purposes only.

Any references cited herein are incorporated by reference in their entireties.

As used herein, the singular form “a,” “an,” and “the” include plural references unless the context clearly dictates otherwise. For example, the term “a compound” or “at least one compound” may include a plurality of compounds, including mixtures thereof. Thus, for example, reference to “plant,” “the plant,” or “a plant” also includes a plurality of plants; also, depending on the context, use of the term “plant” can also include genetically similar or identical progeny of that plant; use of the term “a nucleic acid” optionally includes, as a practical matter, many copies of that nucleic acid molecule.

As used herein, the term “about” indicates that a value includes the inherent variation of error for the method being employed to determine a value, or the variation that exists among experiments.

As used herein, “encoding” refers either to a polynucleotide (DNA or RNA) encoding for the amino acids of a polypeptide or a DNA encoding for the nucleotides of an RNA. As used herein, “coding sequence” and “coding region” are used interchangeably and refer to a polynucleotide that encodes a polypeptide. The boundaries of a coding region are generally determined by a translation start codon at its 5′ end and a translation stop codon at its 3′ end.

As used herein, the term “identity” when used in relation to nucleic acids, describes the degree of similarity between two or more nucleotide sequences. The percentage of “sequence identity” between two sequences can be determined by comparing two optimally aligned sequences over a comparison window, such that the portion of the sequence in the comparison window may comprise additions or deletions (gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. A sequence that is identical at every position in comparison to a reference sequence is said to be identical to the reference sequence and vice-versa. An alignment of two or more sequences may be performed using any suitable computer program. For example, a widely used and accepted computer program for performing sequence alignments is CLUSTALW v1.6 (Thompson, et al. (1994) Nucl. Acids Res., 22: 4673-4680).

As used herein, the terms “nucleic acid,” “polynucleotide,” and “oligonucleotide are used interchangeably and refer to deoxyribonuclotides (DNA), ribonucleotides (RNA), and functional analogues thereof, such as complementary DNA (cDNA) in linear or circular conformation. Nucleic acid molecules provided herein can be single stranded or double stranded. Nucleic acid molecules comprise the nucleotide bases adenine (A), guanine (G), thymine (T), cytosine (C). Uracil (U) replaces thymine in RNA molecules. Analogues of the natural nucleotide bases, as well as nucleotide bases that are modified in the base, sugar, and/or phosphate moieties are also provided herein. The symbol “N” can be used to represent any nucleotide base (e.g., A, G, C, T, or U). The symbol “Y” can be used to represent thymine or cytosine bases. The symbol “V” can be used to represent the nucleotide bases A, C or G. As used herein, “complementary” in reference to a nucleic acid molecule or nucleotide bases refers to A being complementary to T (or U), and G being complementary to C. Two complementary nucleic acid molecules are capable of hybridizing with each other under appropriate conditions. In an aspect of the present disclosure, two nucleic acid sequences are homologous if they have at least 70%, at least 75%, at least 80%, at least 85%, at least 90%, at least 95%, at least 96%, at least 97%, at least 98%, at least 99% or 100% sequence identity with each other.

As used herein, the term “plant” refers to any photosynthetic, cukaryotic, unicellular or multicellular organism of the kingdom Plantae and includes a whole plant or a cell or tissue culture derived from a plant, comprising any of: whole plants, plant components or organs (e.g., leaves, stems, roots, etc.), plant tissues, seeds, plant cells, protoplasts and/or progeny of the same. A progeny plant can be from any filial generation, e.g., F1, F2, F3, F4, F5, F6, F7, etc. A “plant cell” is a biological cell of a plant, taken from a plant or derived through culture from a cell taken from a plant. The term plant encompasses monocotyledonous and dicotyledonous plants. The methods, systems, and compositions described herein are useful across a broad range of plants. Suitable plants in which the methods, systems, and compositions disclosed herein can be used include, but are not limited to, cereals and forage grasses (e.g., alfalfa, rice, maize, wheat, barley, oat, sorghum, pearl millet, finger millet, cool-season forage grasses, and bahiagrass), oilseed crops (e.g., soybean, oilseed brassicas including canola and oilseed rape, sunflower, peanut, flax, sesame, and safflower), legume grains and forages (e.g., common bean, cowpea, pea, faba bean, lentil, tepary bean, Asiatic beans, pigeonpea, vetch, chickpea, lupine, alfalfa, and clovers), temperate fruits and nuts (e.g., apple, pear, peach, plums, berry crops, cherries, grapes, olive, almond, and Persian walnut), tropical and subtropical fruits and nuts (e.g., citrus including limes, oranges, and grapefruit; banana and plantain, pineapple, papaya, mango, avocado, kiwifruit, passionfruit, and persimmon), vegetable crops (e.g., solanaceous plants including tomato, eggplant, and peppers; vegetable brassicas; radish, carrot, cucurbits, alliums, asparagus, and leafy vegetables), sugar cane, tubers (e.g., beets, parsnips, potatoes, turnips, sweet potatoes), and fiber crops (sugarcane, sugar beet, stevia, potato, sweet potato, cassava, and cotton), plantation crops, ornamentals, and turf grasses (tobacco, coffee, cocoa, tea, rubber tree, medicinal plants, ornamentals, and turf grasses), and forest tree species.

As used herein, “plant genome” refers to a nuclear genome, a mitochondrial genome, or a plastid (e.g., chloroplast) genome of a plant cell. In some embodiments, a plant genome may comprise a parental genome contributed by the male and a parental genome contributed by the female. In some embodiments, a plant genome may comprise only one parental genome.

As used herein, “polynucleotide” refers to a nucleic acid molecule containing multiple nucleotides and generally refers both to “oligonucleotides” (a polynucleotide molecule of 18-25 nucleotides in length) and polynucleotides of 26 or more nucleotides. Aspects of this disclosure include compositions including oligonucleotides having a length of 18-25 nucleotides (e. g., 18-mers, 19-mers, 20-mers, 21-mers, 22-mers, 23-mers, 24-mers, or 25-mers), or medium-length polynucleotides having a length of 26 or more nucleotides (e. g., polynucleotides of 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, or about 300 nucleotides), or long polynucleotides having a length greater than about 300 nucleotides (e. g., polynucleotides of between about 300 to about 400 nucleotides, between about 400 to about 500 nucleotides, between about 500 to about 600 nucleotides, between about 600 to about 700 nucleotides, between about 700 to about 800 nucleotides, between about 800 to about 900 nucleotides, between about 900 to about 1000 nucleotides, between about 300 to about 500 nucleotides, between about 300 to about 600 nucleotides, between about 300 to about 700 nucleotides, between about 300 to about 800 nucleotides, between about 300 to about 900 nucleotides, or about 1000 nucleotides in length, or even greater than about 1000 nucleotides in length, for example up to the entire length of a target gene including coding or non-coding or both coding and non-coding portions of the target gene). Where a polynucleotide is double-stranded, its length can be similarly described in terms of base pairs.

As used herein, terms “polypeptide”, “peptide” and “protein” are used interchangeably to refer to a polymer of amino acid residues. The term also applies to amino acid polymers in which one or more amino acids are chemical analogues or modified derivatives of a corresponding naturally-occurring amino acids.

As used herein, “protoplast” refers to a plant cell that has had its protective cell wall completely or partially removed using, for example, mechanical or enzymatic means resulting in an intact biochemical competent unit of living plant that can reform their cell wall, proliferate and regenerate grow into a whole plant under proper growing conditions.

As used herein, a “recombinant nucleic acid” refers to a nucleic acid molecule (DNA or RNA) having a coding and/or non-coding sequence distinguishable from endogenous nucleic acids found in natural systems. In some aspects, a recombinant nucleic acid provided herein is used in any composition, system or method provided herein. In some aspects, a recombinant nucleic acid may encode any CRISPR enzyme provided herein can be used in any composition, system or method provided herein. In some aspects, a recombinant nucleic acid may comprise or encode any guide RNA provided herein can be used in any composition, system or method provided herein. In an aspect, a vector provided herein comprises any recombinant nucleic acid provided herein. In another aspect, a cell provided herein comprises a recombinant nucleic acid provided herein. In another aspect, a cell provided herein comprises a vector provided herein.

As used herein, the term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as meristem, or particular cell types (e.g., pollen). Regulatory elements may also direct expression in a temporal-dependent manner, such as in a cell-cycle dependent or developmental stage-dependent manner, which may or may not also be tissue or cell-type specific. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); and SV40 enhancer.

As used herein, the terms “target sequence” or “target site” refer to a nucleotide sequence against which a guide RNA is capable of hybridizing. A target sequence may be genic or non-genic. In some aspects, a target sequence provided herein comprises a genic region. In other aspects, a target sequence provided herein comprises an intergenic region. In yet another aspect, a target sequence provided herein comprises both a genic region and an intergenic region. In an aspect, a target sequence provided herein comprises a coding nucleic acid sequence. In another aspect, a target sequence provided herein comprises a non-coding nucleic acid sequence. In an aspect, a target sequence provided herein is located in a promoter. In another aspect, a target sequence provided herein comprises an enhancer sequence. In yet another aspect, a target sequence provided herein comprises both a coding nucleic acid sequence and a non-coding nucleic acid sequence. In one aspect, a target sequence provided herein is recognized and cleaved by a double-strand break inducing agent, such as a system comprising a Cpf1 enzyme and a guide RNA.

As used herein, the term “donor” or “donor DNA” means a single stranded or double stranded DNA that comprises a polynucleotide sequence to be inserted at or near the target site of a Cpf1 enzyme and guide system. In some embodiments, the donor DNA comprises a transgene for insertion into the plant cell genome. In some embodiments, the donor DNA comprises a first and a second region of homology that flank the transgene, where the first and second regions of homology share homology to a first and a second genomic region present in or flanking the target site. A region of homology can be of any length that is sufficient to promote homologous recombination at the target site. For example, a region of homology can comprise at least 5-10, 10-15, 15-20, 20-25, 25-30, 30-35, 35-40, 40-45, 45- 50, 50-55, 55-60, 60-65, 65-70, 70-75, 75-80, 80-85, 85-90, 90-95, 95-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1,000, 1,000-1,150, 1,150-1,200, 1,200-1,250, 1,250-1,300, 1,300-1,350, 1,350-1,400, 1,400-1,450, 1,450-1,500, 1,500-1,550,1,550 -1,600, 1,600-1,650, 1,650-1,700, 1,700-1,750, 1,750-1,800, 1,800-1,850, 1,850-1,900, 1,900-1,950, 1,950-2,000, or more bases in length. In some embodiments, the donor DNA comprises a polynucleotide sequence that comprises at least 1, 2, 3, 4, 5, 6, 7, 8, 9, 10 or more nucleotide modifications compared to the target site. In some embodiments, the donor DNA comprises a polynucleotide sequence that is at least 80%, 81%, 82%, 83%, 84%, 85%, 86%, 87%, 88%, 89%, 90%, 91%, 92%, 93%, 94%, 95%, 96%, 97%, 98%, 99% identical to a polynucleotide sequence at or adjacent to the target site. In some embodiments, the donor DNA is 20, 25, 26, 27, 28, 29, 30, 31, 30-35, 35-40, 40-45, 45- 50, 50-55, 55-60, 60-65, 65-70, 70-75, 75-80, 80-85, 85-90, 90-95, 95-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1,000, 1,000-1,150, 1,150-1,200, 1,200-1,250, 1,250-1,300, 1,300-1,350, 1,350-1,400, 1,400-1,450, 1,450-1,500, 1,500-1,550, 1,550-1,600, 1,600-1,650, 1,650-1,700, 1,700-1,750, 1,750-1,800, 1,800-1,850, 1,850-1,900, 1,900-1,950, 1,950-2,000, 2,000-2,100, 2,000-2,200, 2,000-2,300, 2,000-2,400, 2,000-2,500, 2,000-2,600, 2,000-2,700, 2,000-2,800, 2,000-2,900, 2,000-3,000, 3,000-3,100, 3,000-3,200, 3,000-3,300, 3,000-3,400, 3,000-3,500, 3,000-3,600, 3,000-3,700, 3,000-3,800, 3,000-3,900, 3,000-4,000, 4,000-4,100, 4,000-4,200, 4,000-4,300, 4,000-4,400, 4,000-4,500, 4,000-4,600, 4,000-4,700, 4,000-4,800, 4,000-4,900, 4,000-5,000, or more nucleotides in length.

In an aspect, a Cpf1 nuclease provided herein is a Lachnospiraceae bacterium Cpf1 (LbCpf1) nuclease. In another aspect, a Cpf1 nuclease provided herein is a Francisella novicida Cpf1 (FnCpf1) nuclease.

A prerequisite for cleavage of the target site by a CRISPR ribonucleoprotein is the presence of a conserved Protospacer Adjacent Motif (PAM) near the target site. Depending on the CRISPR nuclease, cleavage can occur within a certain number of nucleotides (e.g., between 18-23 nucleotides for Cpf1) from the PAM site. PAM sites are only required for type I and type II CRISPR associated proteins, and different CRISPR endonucleases recognize different PAM sites. Without being limiting, the Cpf1 from Lachnospiraceae bacterium can recognize at least the following PAM sites: TTTN, and YTN; (where T is thymine; Y is thymine or cytosine; and N is thymine, cytosine, guanine, or adenine). Without being limiting, the Cpf1 from Francisella novicida can recognize at least the following PAM sites: TTN (where T is thymine; and N is thymine, cytosine, guanine, or adenine). In certain embodiments, the LbCpf1 protein disclosed here has been modified to recognize a non-natural PAM. LbCpf1 variants comprising one or more amino acid substitutions resulting in altered PAM sequence specificities have been disclosed in the art (for example see Gao et. al., Nature Biotech., 2017 August; 35(8):789-792). Gao et. al. have disclosed two LbCpf1 variants: SEQ ID NO: 39 comprising the amino acid substitutions G532R/K595R that can recognize TYCV PAM (where T is thymine; Y is thymine or cytosine; C is cytosine and V is cytosine, guanine, or adenine) and SEQ ID NO: 76 comprising the amino acid substitutions G532R/K538V/Y542R that can recognize the TATV PAM (where T is thymine; A is adenine; and V is cytosine, guanine, or adenine). As used herein, LbCpf1(TYC) variant refers to an LbCpf1 nuclease comprising the amino acid substitutions G532R/K595R. As used herein, LbCpf1(TAT) variant (SEQ ID NO: 76) refers to an LbCpf1 nuclease comprising the mutations G532R/K538V/Y542R.

The instant disclosure provides a recombinant nucleic acid encoding the Cpf1 nuclease of SEQ ID NO 2, 39, 43, 76 or a fragment thereof, wherein the recombinant nucleic acid is optimized for expression in a plant cell. A sequence can be optimized for expression in a plant cell by modifying a nucleotide sequence encoding a protein such as, for example, the nucleic acid sequence encoding the Cpf1 nuclease of SEQ ID NO 2, 39, 43 or a fragment thereof, using one or more plant-preferred codons for improved expression. In some embodiments, the plant-optimized recombinant nucleic acid encoding the Cpf1 nuclease of SEQ ID NO 2, or a fragment thereof, comprises a sequence having at least 80% identity, at least 81% identity, at least 82% identity, at least 83% identity, at least 84% identity, at least 85% identity, at least 90% identity, at least 91% identity, at least 92% identity, at least 93% identity, at least 94% identity, at least 95% identity, at least 96% identity, at least 97% identity, at least 98% identity, at least 99%, or 100% identity to a sequence selected from SEQ ID NOs: 1 and 10, or a fragment thereof. In some embodiments, the plant-optimized recombinant nucleic acid encoding the LbCpf1(TYC) nuclease (SEQ ID NO: 39), or a fragment thereof, comprises a sequence having at least 80% identity, at least 81% identity, at least 82% identity, at least 83% identity, at least 84% identity, at least 85% identity, at least 90% identity, at least 91% identity, at least 92% identity, at least 93% identity, at least 94% identity, at least 95% identity, at least 96% identity, at least 97% identity, at least 98% identity, at least 99%, or 100% identity to a sequence selected from SEQ ID NOs: 38, or a fragment thereof. In some embodiments, the plant-optimized recombinant nucleic acid encoding the LbCpf1(TAT) nuclease (SEQ ID NO: 76) or a fragment thereof, comprises a sequence having at least 80% identity, at least 81% identity, at least 82% identity, at least 83% identity, at least 84% identity, at least 85% identity, at least 90% identity, at least 91% identity, at least 92% identity, at least 93% identity, at least 94% identity, at least 95% identity, at least 96% identity, at least 97% identity, at least 98% identity, at least 99%, or 100% identity to a sequence selected from SEQ ID NOs: 75, or a fragment thereof.

In some embodiments, the plant-optimized recombinant nucleic acid encoding the FnCpf1 nuclease (SEQ ID NO 43), or a fragment thereof, comprises a sequence having at least 80% identity, at least 81% identity, at least 82% identity, at least 83% identity, at least 84% identity, at least 85% identity, at least 90% identity, at least 91% identity, at least 92% identity, at least 93% identity, at least 94% identity, at least 95% identity, at least 96% identity, at least 97% identity, at least 98% identity, at least 99%, or 100% identity to a sequence selected from SEQ ID NOs: 45-48,50, 51 or a fragment thereof.

In some embodiments, the plant-optimized recombinant nucleic acid is operably linked to a heterologous promoter. In one aspect, a recombinant nucleic acid provided herein comprises one or more, two or more, three or more, four or more, five or more, six or more, seven or more, eight or more, nine or more, or ten or more heterologous promoters operably linked to one or more, two or more, three or more, four or more, five or more, six or more, seven or more, eight or more, nine or more, or ten or more plant-optimized recombinant nucleic acids encoding a Cpf1 nuclease. In some embodiments, a plant-optimized recombinant nucleic acids encoding a Cpf1 nuclease provided herein is provided to a plant cell in combination with one or more, two or more, three or more, four or more, five or more, six or more, seven or more, eight or more, nine or more, or ten or more guide polynucleotides. As used herein, the term “guide polynucleotide” refers to a polynucleotide sequence that can form a complex with a Cpf1 endonuclease and enables the Cpf1 endonuclease to bind to, and optionally cleave, a target site. The guide polynucleotide sequence can be a RNA sequence, a DNA sequence, or any combination thereof (e.g., a RNA-DNA hybrid sequence). In one aspect, a guide polynucleotide provided herein comprises a CRISPR repeat sequence and a spacer sequence that is complementary to a target site. In one aspect, a guide polynucleotide provided herein comprises one or more repeats of a CRISPR repeat sequence, a spacer sequence, and a CRISPR repeat sequence. In some embodiments, the guide polynucleotide comprises two or more spacer sequences that are complementary to different target sites. In some embodiments, the guide polynucleotide comprises one or more CRISPR repeat sequences selected from a pre-crRNA and a mature cr-RNA. In some embodiments, the guide polynucleotide is operably linked to a promoter. In certain embodiments, recombinant nucleic acids encoding guide polynucleotides may be designed in an array format such that multiple guide polynucleotides can be simultaneously released. In some embodiments, expression of one or more guide polynucleotides is U6-driven. In some embodiments, Cpf1 enzymes complex with multiple guide polynucleotides to mediate genome editing and at multiple target sequences. Some embodiments relate to expression of singly or in tandem array format from 1 up to 4 or more different guide sequences; e.g. up to about 20 or about 30 guides sequences. Each individual guide sequence may target a different target sequence. Such may be processed from, e.g. one chimeric pol3 transcript. Pol3 promoters such as U6 or H1 promoters may be used.

In some embodiments, a plant-optimized recombinant nucleic acid as disclosed herein is expressed or delivered in a vector. As used herein, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is an Agrobacterium T-DNA. Another type of vector is a viral vector, wherein virally-derived DNA or RNA sequences are present in the vector for packaging into a virus (e.g., retroviruses, replication defective retroviruses, Tobacco mosaic virus (TMV), Potato virus X (PVX) and Cowpea mosaic virus (CPMV), tobamovirus, Gemini viruses, adenoviruses, replication defective adenoviruses, and adeno-associated viruses). Viral vectors also include polynucleotides carried by a virus for transfection into a host cell. In some embodiments, a viral vector may be delivered to a plant using Agrobacterium. Certain vectors are capable of autonomous replication in a host cell into which they are introduced. Other vectors are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.). In some embodiments, an expression vector can comprise a plant-optimized recombinant nucleic acid in a form suitable for expression of the plant-optimized recombinant nucleic acid in a plant cell, which means that the expression vector comprises one or more regulatory elements that are operatively-linked to the plant-optimized recombinant nucleic acid to be expressed. Regulatory elements may include enhancers, termination sequences, introns, etc.

In certain embodiments, the plant-optimized recombinant nucleic acid may be operably linked to a nucleic acid sequence encoding one or more nuclear localization signal (NLS), nuclear export signal (NES), functional domains, and flexible linkers. The one or more of the NLS, the NES or the functional domain may be conditionally activated or inactivated. In particular embodiments it can be of interest to target the Cpf1 encoded by the plant-optimized recombinant nucleic acid to the chloroplast. In many cases, this targeting may be achieved by the operably linking the plant-optimized recombinant nucleic acid encoding Cpf1 to a nucleic acid encoding a chloroplast transit peptide (CTP) or plastid transit peptide. Other options for targeting to the chloroplast which have been described are the maize cab-m7 signal sequence (U.S. Pat. No. 7,022,896, WO 97/41228, incorporated by reference herein) a pea glutathione reductase signal sequence (WO 97/41228, incorporated by reference herein) and the CTP described in US2009029861, incorporated by reference herein.

Several embodiments relate to a method for modifying a target sequence in the genome of a plant cell, the method comprising: introducing a recombinant nucleic acid optimized for expression in a plant cell comprising one or more of SEQ ID NOs: 1, 4, 6, 10, 12, 14, 15, 26, 31, 36, 38, 40, 41, 45, 46, 47, 48, 49, 50, 51, 63 ,65, 66, 67, 68, 68, 70, 71, 72, 73, and 75 and a guide polynucleotide comprising a targeting domain that is complementary to a target sequence into the plant cell, where the recombinant nucleic acid expresses Cpf1 endonuclease in the plant cell and the Cpf1 endonuclease and the guide polynucleotide are capable of forming a complex that can recognize, bind to, and optionally nick or cleave the target sequence. In some embodiments, the guide polynucleotide and/or the recombinant nucleic acid are introduced into the plant cell by biolistic delivery. Several embodiments relate to a method for modifying a target sequence in the genome of a plant cell, the method comprising: introducing a guide polynucleotide comprising a targeting domain that is complementary to a target sequence in the plant genome into a plant cell comprising a recombinant nucleic acid optimized for expression in a plant cell, wherein the recombinant nucleic acid comprises one or more of SEQ ID NOs: 11, 4, 6, 10, 12, 14, 15, 26, 31, 36, 38, 40, 41, 45, 46, 47, 48, 49, 50, 51, 63 ,65, 66, 67, 68, 68, 70, 71, 72, 73, and 75 where the recombinant nucleic acid expresses Cpf1 endonuclease in the plant cell and the Cpf1 endonuclease and the guide polynucleotide are capable of forming a complex that can recognize, bind to, and optionally nick or cleave the target sequence. In some embodiments, the guide polynucleotide is introduced into the plant cell by biolistic delivery. In some embodiments, the method further comprises incubating the plant cell at temperatures between 24° C. and 25° C., 25° C. and 26° C., 26° C. and 27° C., 27° C. and 28° C., 28° C. and 29° C., 29° C. and 30° C., 30° C. and 31° C., 31° C. and 32° C., 32° C. and 33° C., 33° C. and 34° C., 34° C. and 35° C., 35° C. and 36° C., 36° C. and 37° C., 37° C. and 38° C., 38° C. and 39° C., 39° C. and 40° C., for a period of at least about 10 min., 15 min., 20 min., 25 min., 30 min., 35 min., 40 min., 45 min., 50 min., 55 min., 1 hr., 2 hrs., 3 hr., 4 hrs., 5 hrs., 6 hrs., 7 hrs., 8 hrs., 9 hrs., 10 hrs., 11 hrs., 12 hrs., 13 hrs., 14 hrs., 15 hrs., 16 hrs., 17 hrs., 18 hrs, 19 hrs., 20 hrs. 21 hrs., 22 hrs., 23 hrs., 24 hrs., 25 hrs., 26 hrs., 27 hrs., 28 hrs., 29 hrs., 30 hrs., 31 hrs., 32 hrs., 33 hrs., 34 hrs., 35 hrs., 36 hrs., 37 hrs., 38 hrs., 39 hrs., 40 hrs., 41 hrs., 42 hrs., 43 hrs. 44 hrs., 45 hrs., 46 hrs., 47 hrs., 48 hrs., 3 days, 4 days, 5 days, 6 days, or 7 days. In some embodiments, the methods described herein can further comprise identifying at least one plant cell, plant or progeny plant that has a modification at the target sequence, where the modification at the target sequence is selected from the group consisting of (i) a replacement of at least one nucleotide, (ii) a deletion of at least one nucleotide, (iii) an insertion of at least one nucleotide, and (iv) any combination of (i)-(iii). The method can further provide a donor DNA to the plant cell, where the donor DNA comprises a polynucleotide sequence of interest. This can produce a plant cell or plant having a detectable targeted genome modification.

Several embodiments relate to a method for modifying a target sequence in the genome of a plant cell, method comprising: obtaining a plant cell comprising in its genome a recombinant nucleic acid comprising a sequence selected from the group consisting of: SEQ ID NOs 1, 4, 6, 10, 12, 14, 15, 26, 31, 36, 38, 40, 41, 45, 46, 47, 48, 49, 50, 51, 63 ,65, 66, 67, 68, 68, 70, 71, 72, 73, and 75 and introducing into the plant cell a guide polynucleotide comprising a targeting domain that is complementary to a target sequence in the plant genome or a recombinant nucleic acid encoding the guide polynucleotide, where the guide polynucleotide and Cpf1 endonuclease encoded by the recombinant nucleic acid are capable of forming a complex that can bind to, and modify the target sequence. In some embodiments, the guide polynucleotide is introduced into the plant cell by biolistic delivery. In some embodiments, the method further comprises incubating the plant cell at temperatures between 24° C. and 25° C. 25° C. and 26° C., 26° C. and 27° C., 27° C. and 28° C., 28° C. and 29° C., 29° C. and 30° C., 30° C. and 31° C., 31° C. and 32° C., 32° C. and 33° C., 33° C. and 34° C., 34° C. and 35° C., 35° C. and 36° C., 36° C. and 37° C., 37° C. and 38° C., 38° C. and 39° C., 39° C. and 40° C., for a period of at least about 10 min., 15 min., 20 min., 25 min., 30 min., 35 min., 40 min., 45 min., 50 min., 55 min., 1 hr., 2 hrs., 3 hr., 4 hrs., 5 hrs., 6 hrs., 7 hrs., 8 hrs., 9 hrs., 10 hrs., 11 hrs., 12 hrs., 13 hrs., 14 hrs., 15 hrs., 16 hrs., 17 hrs., 18 hrs, 19 hrs., 20 hrs. 21 hrs., 22 hrs., 23 hrs., 24 hrs., 25 hrs., 26 hrs., 27 hrs., 28 hrs., 29 hrs., 30 hrs., 31 hrs., 32 hrs., 33 hrs., 34 hrs., 35 hrs., 36 hrs., 37 hrs., 38 hrs., 39 hrs., 40 hrs., 41 hrs., 42 hrs., 43 hrs. 44 hrs., 45 hrs., 46 hrs., 47 hrs., 48 hrs., 3 days, 4 days, 5 days, 6 days, or 7 days. In some embodiments, the methods described herein can further comprise identifying at least one plant cell, plant or progeny plant that has a modification at the target sequence, where the modification at the target sequence is selected from the group consisting of (i) a replacement of at least one nucleotide, (ii) a deletion of at least one nucleotide, (iii) an insertion of at least one nucleotide, and (iv) any combination of (i)-(iii). The method can further provide a donor DNA to the plant cell, where the donor DNA comprises a polynucleotide sequence of interest. This can produce a plant cell or plant having a detectable targeted genome modification.

The plant cell may be of a monocot or dicot. In some embodiments, the plant cell may be from or of a crop or grain plant such as cassava, corn, sorghum, alfalfa, cotton, soybean, canola, wheat, oat or rice. The plant cell may also be of an algae, tree or production plant, fruit or vegetable (e.g., trees such as citrus trees, e.g., orange, grapefruit or lemon trees; peach or nectarine trees; apple or pear trees; nut trees such as almond or walnut or pistachio trees; nightshade plants; plants of the genus Brassica; plants of the genus Lactuca; plants of the genus Spinacia; plants of the genus Capsicum; cotton, tobacco, asparagus, avocado, papaya, cassava, carrot, cabbage, broccoli, cauliflower, tomato, eggplant, pepper, lettuce, spinach, strawberry, potato, squash, melon, blueberry, raspberry, blackberry, grape, coffee, cocoa, etc).

The methods for genome editing using the recombinant nucleic acid molecules as described herein can be used to confer desired traits on essentially any plant. A wide variety of plants and plant cell systems may be engineered for the desired physiological and agronomic characteristics described herein using the nucleic acid constructs of the present disclosure and the various transformation methods mentioned above.

### EXAMPLES

The following examples are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); ss, single stranded; ds, double stranded and the like.

### Example 1

### Design and Analysis of LbCpf1-CO1, an Engineered Polynucleotide Optimized for Expression in Plant Cells.

This example describes the creation and testing of a synthetic polynucleotide encoding Lachnospiraceae bacterium ND2006 (LbCpf1) nuclease that is optimized for expression in plant cells.

A nucleotide sequence of Cpf1 from Lachnospiraceae bacterium ND2006 (LbCpf1) that was codon optimized for expression in human cells has been described by Zetsche et. al, (Cell 2015, 163, 759-771). The human codon optimized sequence disclosed by Zetsche et. al., was modified through algorithmic methods, partly based on corn codon preference, to design LbCpf1-CO1(Coding sequence Optimized version 1) (SEQ ID NO: 1) to optimize the sequence for expression of the LbCpf1 protein (SEQ ID NO: 2) in plant cells.

The plant-optimized LbCpf1-CO1 sequence was then incorporated into six different expression vectors to test its activity in corn cells. Three of the expression vectors were designed with an expression cassette (SEQ ID NO: 3) comprising the LbCpf1-CO1 nuclease and a nucleotide sequence encoding the Nuclear Localization Sequence (NLS) from the heat stress transcription factor 1 (HSFA1) gene from Solanum lycopersicum (SEQ ID NO:4) on the 5′ and 3′ ends. Three of the expression vectors were designed with an expression cassette (SEQ ID NO:5) comprising a processable potato LS1 intron sequence (SEQ ID NO: 6) introduced into the NLS-LbCpf1-CO1-NLS sequence to eliminate expression of the LbCpf1protein in Agrobacterium. The NLS-LbCpf1-CO1-NLS expression cassettes also comprised a Zea mays Ubiquitin M1 promoter leader and intron sequence (SEQ ID NOs:7) operably linked to the NLS-LbCpf1-CO1-NLS nuclease and a transcription terminator sequence from a rice Lipid transfer protein (LTP) gene (SEQ ID NO:8). Each plant vector also comprised a gRNA expression array comprising either 2 or 4 guide RNA sequences (mature crRNA+spacer) positioned in tandem and targeting 2 or 4 sites in a corn chromosome. The first crRNA sequence was 35 nt while the remaining ones were 20 nt and the spacer sequence was 30 nt. The gRNA arrays were operably linked to the maize U6 Pol III promoter (SEQ ID NO:9) and a poly T terminator sequence. All the expression vectors also included a third expression cassette containing the selectable marker gene CP4 that provides resistance to the herbicide glyphosate. See Table 1.

TABLE 1
SUMMARY OF RESULTS OF FRAGMENT LENGTH ANALYSIS (FLA)
GENERATED FROM CORN PLANTS STABLY TRANSFORMED WITH
LBCPF1-CO1 AND GRNAS TARGETING 8 UNIQUE GENOMIC TARGET SITES.

| | Intron | | | | | |
|---|---|---|---|---|---|---|
| | in Cpf1- | | | Plants | # Edited | Mutation |
| | CO1 | Genomic sites | Plants | returning | samples by | efficiency |
| Vector | cassette | targeted (TS) | tested | data | FLA | (%) |
| 1 | No | ZmTS1, ZmTS2 | 47 | 34 | 0 | 0 |
| 2 | No | ZmTS3, ZmTS4, | 55 | 50 | 0 | 0 |
| | | ZmTS5, ZmTS6 | | | | |
| 3 | No | ZmTS7, ZmTS8 | 45 | 44 | 0 | 0 |
| 4 | Yes | ZmTS7, ZmTS8 | 38 | 37 | 1 | 2.63 |
| 5 | Yes | ZmTS1, Zm TS2 | 65 | 64 | 0 | 0 |
| 6 | Yes | ZmTS3, ZmTS4, | 35 | 29 | 0 | 0 |
| | | ZmTS5, ZmTS6 | | | | |
| Total | | | 285 | 258 | 1 | |

Corn 01DKD2 cultivar embryos were transformed with agrobacterium containing the plant expression vectors described in Table 1. Transformed plants were selected on glyphosate, leaf samples from regenerated plantlets were harvested after 2 weeks and genomic DNA was extracted for Fragment Length Analysis (FLA). FLA is a PCR-based molecular assay that can be used to identify indel (insertion or deletion) mutations introduced at the target site by NHEJ-mediated (Non Homologous End Joining) DNA repair following dsDNA cleavage by the Cpf1-guide complex. Genomic DNA was subjected to a PCR reaction with primers flanking the target site to generate amplicons. The amplicons fragment length was then compared to a wild type amplicon to identify mutants. PCR reactions were carried out using 5′ FAM-labeled primer, a standard primer and Phusion™ polymerase (New England Biolabs, MA) according to manufactures instructions to generate 200 to 500 bp PCR fragments. 1 ul PCR product was combined with 0.5 ul GeneScan 1200 LIZ Size Standard (Thermo Fisher, MA), 8.5 ul formamide and run on ABI sequencer (Thermo Fisher, MA). Two FLA reactions were multiplexed and subsequently analyzed for fragment length variation to identify plants with mutations at the target sites. As shown in Table 1, 258 plants returned high quality FLA data, out of which only 1 plant was identified as having mutations at one of the target sites.

### Example 2

### Design and Analysis of LbCpf1-CO2, an Engineered Polynucleotide Optimized for Expression in Plant Cells.

This example describes the design and expression analysis of Lachnospiraceae bacterium ND2006 (LbCpf1) nuclease that is optimized for expression in plant cells.

The LbCpf1-CO1 nucleotide sequence described in Example 1 was manually analyzed for the presence of deleterious motifs that could potentially reduce gene expression. These deleterious motifs were given a higher priority for removal/replacement by nucleotide sequences coding for synonymous codons. Additionally, a monocot-specific codon frequency table was used for optimization of the nucleotide sequence for expression in monocots. Based on these criteria, a second optimized LbCpf1 (referred to as LbCpf1-CO2) nucleotide sequence was generated (SEQ ID NO: 10) for expression of the LbCpf1 protein (SEQ ID NO: 2) in planta. When compared to LbCpf1-CO1, the LbCpf1-CO2 sequence was determined to have a threefold reduction in the presence of deleterious motifs within its coding sequence. The full length LbCpf1-CO2 nucleotide sequence shows only 85.6% sequence identity with the human codon optimized LbCpf1 nucleotide sequence disclosed by Zetsche et. al., (Cell 2015, 163, 759-771), only 77.5% sequence identity with LbCpf1-CO1 and only 69.4% sequence identity with the native bacterial LbCpf1 sequence.

Three expression cassettes (Prom35S::HIStag:NLS:LbCpf1-CO2:mOrange:NLS::TermNOS; Prom35S::HIStag:NLS:LbCpf1-Os:mOrange:NLS::TermNOS; and Prom_{35S}::HIStag:NLS:mOrange:NLS::Term_{NOS}) were generated by standard cloning techniques and as described below:
(1) Prom35S::HIS tag:NLS:LbCpf1-CO2:mOrange:NLS::TermNOS

The LbCpf1-CO2 coding sequence was fused 5′ to the coding sequence of mOrange (mOr) from Entacmaea quadricolor (SEQ ID NO: 52). The LbCpf1-CO2:mOrange fusion gene was then flanked at the 5′ and 3′ ends with the NLS sequence from tomato HSFA1 gene (SEQ ID NO:3) and a nucleotide sequence encoding a HIS tag (MGSS7H) (SEQ ID NO: 54) was introduced to the 5′ end. The nucleotide sequence was operably linked to the Cauliflower mosaic virus 35S promoter and an Agrobacterium NOS terminator.
(2) Prom_{35S}::HIS tag:NLS:LbCpf1-Os:mOrange:NLS::Term_{NOS}

The rice codon-optimized Cpf1 (LbCpf1-Os) nucleotide sequence described by Xu et. al. (Plant Biotechnology Journal, 2017, 15, 713-717) (SEQ ID NO: 11) was used as a control to compare in planta expression. The LbCpf1-Os coding sequence was fused 5′ to the coding sequence of mOrange (mOr) from Entacmaea quadricolor (SEQ ID NO: 52). The LbCpf1-Os:mOrange fusion gene was then flanked at the 5′ and 3′ ends with the NLS sequence from tomato HSFA1 gene (SEQ ID NO:3) and a nucleotide sequence encoding a HIS tag (MGSS7H) (SEQ ID NO:54) was introduced to the 5′ end. The nucleotide sequence was operably linked to the Cauliflower mosaic virus 35S promoter and an Agrobacterium NOS terminator.
(3) Prom_{35S}::HIS tag:NLS:mOrange:NLS::Term_{NOS}

The coding sequence of mOrange (mOr) gene (SEQ ID NO:52) from Entacmaea quadricolor was flanked at the 5′ and 3′ ends with the NLS sequence from tomato HSFA1 gene (SEQ ID NO:3) and a nucleotide sequence encoding a HIS tag (MGSS7H) (SEQ ID NO:54) was introduced to the 5′ end. The nucleotide sequence was operably linked to the Cauliflower mosaic virus 35S promoter and an Agrobacterium NOS terminator.

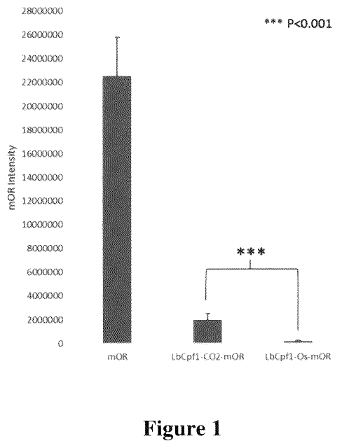

The expression cassettes described above were cloned into plant expression constructs. Corn leaf protoplasts were transfected with either the LbCpf1-CO2-mOr construct, the LbCpf1-Os-mOr construct, or the control mOr construct to evaluate expression levels (Table 2). Since mOrange was fused to LbCpf1-CO2 and LbCpf1-Os, the relative mOrange fluorescence levels reflects LbCpf-CO2 and LbCpf1-Os expression levels. Transformations were carried out using standard polyethylene glycol (PEG) based transfection methods. To quantify transformation frequency, an expression vector comprising the luciferase gene was co-transfected. Following transformation, the protoplasts were incubated in the dark in incubation buffer and harvested after 48 hours. Transformation efficiency was calculated by quantifying luciferase expression. The average mOrange expression from 3 technical replicates was determined using Operetta™ (Perkin Elmer) analysis software. As shown in FIG. 1 and Table 2, mOrange intensity was significantly higher in protoplasts expressing LbCpf1-CO2-mOrange than in cells expressing LbCpf1-Os-mOrange.

TABLE 2
EXPRESSION ANALYSIS OF LBCPF1-CO2-MOR AND LBCPF1-OS-
MOR FLUORESCENT PROTEINS IN CORN PROTOPLASTS

| | | Fold |
|---|---|---|
| | | increase in |
| | | expression |
| | Fluo- | compared |
| | rescence | to Cpf1- |
| Expression Construct | detected | Os-mOr |
| Prom35S::HIStag:NLS:LbCpf1- | Yes | 14 |
| CO2:mOrange:NLS::TermNOS | | |
| Prom35S::HIStag:NLS:LbCpf1- | Yes | 1 |
| Os:mOrange:NLS::TermNOS | | |
| Prom_{35S}:: HIS tag:NLS:mOrange:NLS::Term_{NOS} | Yes | 135 |

### Example 3

### Analysis of LbCpf1-CO2 Activity in Corn Plants.

This example describes testing the LbCpf1-CO2 nucleotide sequence for activity at multiple genomic sites in corn plants using multiplexed guide RNAs.

An agrobacterium LbCpf1-CO2 T-DNA vector comprising: an expression cassette for a selectable marker conferring resistance to the herbicide glyphosate; an expression cassette (SEQ ID NO:15) comprising NLS-LbCpf1-CO2-NLS (SEQ ID NO: 12) linked to a 5′ Kozak sequence (SEQ ID NO:13) resulting in Koz-NLS-LbCpf1-CO2-NLS (SEQ ID NO: 14), which was operably linked to a Zea mays Ubiquitin M1 promoter cassette (SEQ ID NOs:7) and the transcription terminator sequence from rice LTP (SEQ ID NO:8); and an expression cassette comprising the Zea mays U6 promoter (SEQ ID NO:9) and a polyT terminator operably linked to gRNA expression array comprising three gRNAs positioned in tandem and targeting the three genomic sites, was created. Each gRNA comprised a 21 bp crRNA sequence linked to a 23 bp spacer sequence that was complementary to target site ZmTS9, ZmTS10 or ZmTS11 in the corn genome.

As a control, an agrobacterium LbCpf1-Os T-DNA vector comprising: an expression cassette for a selectable marker conferring resistance to the herbicide glyphosate; an expression cassette (SEQ ID NO: 18) comprising a Kozak sequence immediately upstream of the coding sequence of LbCpf1-Os (SEQ ID NO: 11) fused to the tomato HSFA NLS (SEQ ID NO:3) at the 5′ end and the 3′ end which was operably linked to the Zea mays Ubiquitin M1 promoter cassette (SEQ ID NO: 7) and to the rice LTP terminator (SEQ ID NO: 8); and an expression cassette comprising the Zea mays U6 promoter (SEQ ID NO:9) operably linked to gRNA expression array comprising three gRNAs positioned in tandem and targeting the three genomic sites, was created. Each gRNA comprised a 21 bp crRNA sequence linked to a 23bp spacer sequence that was complementary to target site ZmTS9, ZmTS10 or ZmTS11 in the corn genome.

Corn 01DKD2 cultivar embryos were transformed with either the LbCpf1-CO2 or LbCpf1-Os T-DNA vectors described above by agrobacterium-mediated transformation. Transformed plants were selected on glyphosate, leaf samples from regenerated plantlets were harvested after 2 weeks and genomic DNA was extracted for Fragment Length Analysis (FLA) to determine genome mutation rates, as described in Example 1. Table 3 summarizes the results and shows the mutation rate detected at each site in stably transformed corn plants.

TABLE 3
SUMMARY OF RESULTS OF FLA GENERATED FROM CORN
PLANTS STABLY TRANSFORMED WITH EITHER LBCPF1-CO2
OR LBCPF1-OS AND GRNA ARRAY TARGETING 3 UNIQUE
GENOMIC TARGET SITES

| T-DNA | | Plants | # Edited plants | Mutation |
|---|---|---|---|---|
| Vector | Target site | assayed | by FLA analysis | frequency |
| LbCpf1-CO2 | ZmTS9 | 48 | 20 | 41.6% |
| | ZmTS10 | 47 | 6 | 12.7% |
| | ZmTS11 | 46 | 2 | 4.3% |
| LbCpf1-Os | ZmTS9 | 49 | 4 | 8% |
| | ZmTS10 | 49 | 1 | 2% |
| | ZmTS11 | 47 | 2 | 4.3% |

As shown in Table 3, all three sites targeted for cleavage with the guide/LbCpf1-CO2 system described above exhibited the presence of mutations which is indicative of DNA cleavage and repair. The frequency of mutations at the three sites ranged from 4.3% at ZmTS11, 12.7% for ZmTS10 to almost 42% at ZmTS9. 20 plants identified as having mutations in ZmTS9 were further analyzed to confirm the presence of mutations at the target site. PCR primers flanking the target site were used to generate amplicons which were cloned via Zero blunt-end Topo™M cloning (LifeTechnologies), sequenced and compared to the reference sequence. The presence of mutations was confirmed in all 20 events. For the guide/LbCpf1-Os system, mutations were identified at all three sites and the frequency of mutations at the three sites ranged from 2% at TS10, 4.3% for TS11 to almost 9% at TS1. Taken together, the data shows that the plant coding sequence optimized LbCpf1-CO2 is properly transcribed and translated in the corn host cell, is functional and can successfully promote gRNA directed chromosomal cleavage at target sites.

### Example 4

Analysis of LbCpf1-CO2 Activity in Combination with a Single gRNA Expression System in Corn Plants.

This example describes the testing the LbCpf1-CO2 nucleotide sequence for the ability to induce cleavage and subsequent edits at a genomic target site in corn plants utilizing a single gRNA expression cassette.

An agrobacterium T-DNA vector comprising: an expression cassette for a selectable marker gene that conferred resistance to the herbicide glyphosate; an expression cassette (SEQ ID NO:15) comprising a Kozak sequence introduced 5′ to the NLS-LbCpf1-CO2-NLS nucleotide sequence and operably linked to a Zea mays Ubiquitin M1 promoter cassette and the transcription terminator sequence from rice LTP; and an expression cassette comprising the Zea mays U6 Pol III promoter (SEQ ID NO: 9) and a poly T terminator operably linked to a single guide RNA (gRNA) comprising a crRNA sequence linked to a 23 bp spacer sequence complementary to a unique target site (ZmTS12) in the corn chromosome.

Corn 01DKD2 cultivar embryos were transformed with agrobacterium containing the T-DNA vector and stably transformed plants were selected on glyphosate. Leaf samples from regenerated plantlets were harvested after 2 weeks and genomic DNA was extracted for Fragment Length Analysis (FLA) to determine genome mutation rates, as described in Example 1.

TABLE 4
FLA RESULTS GENERATED FROM CORN PLANTS
STABLY TRANSFORMED WITH LBCPF1-CO2 AND
GRNA TARGETING ZMTS12 GENOMIC TARGET SITE.

| | | | # Edited | |
|---|---|---|---|---|
| Nuclease | | | plants by FLA | Mutation |
| sequence | Target site | Plants assayed | analysis | frequency |
| LbCpf1-CO2 | ZmTS12 | 247 | 158 | 64% |

As shown in Table 4, mutations were identified at the target site in 64% of corn plants stably transformed with a vector comprising the LbCp1-CO2 nucleotide sequence and a single guide RNA.

### Example 5

### Analysis of the Effect of the Addition of a Kozak Fragment Upstream of the LbCpf1-Os Nucleotide Sequence on Nuclease Activity in Plant Cells.

This example describes testing the addition of the Kozak sequence (SEQ ID NO:15) upstream of the LbCpf1-Os nucleotide sequence for the ability to enhance nuclease activity in corn plants.

An agrobacterium LbCpf1-Os (Kozak minus) T-DNA vector comprising: an expression cassette for a selectable marker conferring resistance to the herbicide glyphosate; an expression cassette (SEQ ID NO:19) comprising NLS-LbCpf1-Os-NLS (SEQ ID NO:16), with an ATG sequence incorporated immediately 5′ to SEQ ID NO: 16 and operably linked to a Zea mays Ubiquitin M1 promoter cassette (SEQ ID NOs:7) and the transcription terminator sequence from rice LTP (SEQ ID NO:8); and an expression cassette comprising the Zea mays U6 promoter (SEQ ID NO:9) operably linked to gRNA expression array comprising three gRNAs positioned in tandem and targeting the three genomic sites, was created. Each gRNA comprised a 21 bp crRNA sequence linked to a 23 bp spacer sequence that was complementary to target site ZmTS9, ZmTS10 or ZmTS11 in the corn genome.

Corn plants were transformed with agrobacterium containing either the T-DNA vector described above comprising the LbCpf1-Os (Kozak minus) expression cassette (SEQ ID NO:19) or the T-DNA vector described in Example 3 comprising a Kozak sequence immediately upstream of the coding sequence of LbCpf1-Os (SEQ ID NO:18). Transformed plants were selected on glyphosate, leaf samples from regenerated plantlets were harvested after 2 weeks and genomic DNA was extracted for Fragment Length Analysis (FLA) to determine genome mutation rates, as described in Example 1. Table 5 summarizes the results and shows the mutation rate for each site in stably transformed corn plants.

TABLE 5
SUMMARY OF FLA RESULTS GENERATED FROM CORN
PLANTS STABLY TRANSFORMED WITH LBCPF1-OS AND
GRNA ARRAY TARGETING 3 UNIQUE GENOMIC TARGET SITES

| | | | | # Edited | |
|---|---|---|---|---|---|
| | | | | plants by | |
| Kozak | Nuclease | | Plants | FLA | Mutation |
| sequence | sequence | Target site | assayed | analysis | frequency |
| + | LbCpf1-Os | ZmTS9 | 49 | 4 | 8% |
| | | ZmTS10 | 49 | 1 | 2% |
| | | ZmTS11 | 47 | 2 | 4.3% |
| − | LbCpf1-Os | ZmTS9 | 33 | 0 | 0% |
| | | ZmTS10 | 35 | 0 | 0% |
| | | ZmTS11 | 35 | 0 | 0% |

Plants transformed with the LbCpf1-Os comprising a Kozak sequence upstream of the nuclease coding sequence exhibited mutations at all three target sites at frequency ranging from 2% at ZmTS10, 4.3% for ZmTS11 to almost 8% at ZmTS9. No mutants were identified at any of the three target sites in plants transformed with the LbCpf1-Os expression cassette lacking the Kozak sequence.

### Example 6

### Analysis of LbCpf1-CO2 Activity in Soybean Plants.

This example describes testing the LbCpf1-CO2 nucleotide sequence for activity in soybean plants by assaying the ability of the nuclease to target cleavage at multiple unique genomic sites using multiplexed guides.

An agrobacterium LbCpf1-CO2 T-DNA vector was created comprising: an expression cassette for a selectable marker conferring resistance to the antibiotic spectinomycin; an expression cassette (SEQ ID NO: 20) comprising NLS-LbCpf1-CO2-NLS (SEQ ID NO:12) with ATGGCG fused in frame 5′ to SEQ ID NO 12 as the translational start site, which was operably linked to a promoter sequence (SEQ ID NO:37) and a transcriptional terminator sequence from Medicago truncatula (disclosed in US20140283200); and an expression cassette comprising the Glycine max U6 Pol III promoter (disclosed in US20170166912) and a polyT terminator operably linked to a gRNA array comprising three gRNAs arranged in tandem and a transcriptional terminator sequence. Each gRNA comprised a 21 bp mature crRNA sequence linked to a 23 bp spacer sequence that was complementary to either the GmFAD2-1A-TS, GmPDS-TS1 or GmPDS-TS2 target site.

An agrobacterium LbCpf1-Os T-DNA control vector was created comprising: an expression cassette for a selectable marker conferring resistance to the antibiotic spectinomycin; an expression cassette (SEQ ID NO: 21) comprising NLS-LbCpf1-Os-NLS with ATGGCG fused in frame 5′ as the translational start site, which was operably linked to a promoter sequence (SEQ ID NO:37) and a transcriptional terminator sequence from Medicago truncatula (disclosed in US20140283200); and an expression cassette comprising the Glycine max U6 Pol III promoter (disclosed in US20170166912) and polyT terminator operably linked to a gRNA array comprising three gRNAs arranged in tandem and a transcriptional terminator sequence. Each gRNA comprised a 21bp mature crRNA sequence linked to a 23 bp spacer sequence that was complementary to either the GmFAD2-1A-TS, GmPDS-TS1 or GmPDS-TS2 target site.

Excised embryos from A3555 soybean plants were co-cultured with the Agrobacterium containing either the LbCpf1-CO2 T-DNA vector or the LbCpf1-Os T-DNA control vector described above. Transformed plants were selected on spectinomycin, leaf samples from regenerated plantlets were harvested after 4 weeks, and genomic DNA was extracted for Fragment Length Analysis (FLA) to determine genome mutation rates, as described in Example 1. A summary of FLA results generated from soy plants stably transformed with either LbCpf1-CO2 or LbCpf1-Os and gRNA array targeting 3 unique genomic target sites is provided in Table 6.

The plants were also scored for the albino phenotype typically associated with reduction/loss of PDS gene function (Table 7). PDS catalyzes a rate-limiting step in the biosynthesis of carotenoids in plants (Misawa, et. al., The Plant Journal, 1993, 4; 833-840). Reducing the endogenous PDS gene expression will therefore result in plants with a bleached phenotype and lowered chlorophyll content. Presence of an albino phenotype is therefore indicative of mutations at the PDS locus.

TABLE 6
SUMMARY OF FLA RESULTS GENERATED FROM SOY
PLANTS STABLY TRANSFORMED WITH EITHER LBCPF1-CO2
OR LBCPF1-OS AND GRNA ARRAY TARGETING 3 UNIQUE
GENOMIC TARGET SITES

| | | | # Edited | |
|---|---|---|---|---|
| Nuclease seq | | | plants by | Mutation |
| variant | Target sites | Plants assayed | FLA | rate |
| LbCpf1-CO2 | GmFAD2-1A | 62 | 22 | 36% |
| | GmPDS-TS1 | 62 | 0 | 0% |
| | GmPDS-TS2 | 62 | 28 | 45% |
| LbCpf1-Os | GmFAD2-1A | 88 | 20 | 22% |
| | GmPDS-TS1 | 88 | 0 | 0% |
| | GmPDS-TS2 | 88 | 37 | 42% |

TABLE 7
SUMMARY OF PLANTS SCORED FOR PDS GENE
MUTATIONS INDICATED BY AN ALBINO PHENOTYPE.

| | | | Albino frequency |
|---|---|---|---|
| Nuclease | Plants assayed | Albino plants | rate |
| LbCpf1-CO2 | 62 | 52 | 84% |
| LbCpf1-Os | 88 | 60 | 68% |

As summarized in Table 6, of the 3 sites targeted by LbCpf1-Os and LbCpf1-CO2, soybean plants were recovered where mutations were identified at FAD2 and PDS1-TS2 sites. The mutations at the PDS locus was further confirmed by scoring for the albino phenotype (see Table 7).

### Example 7

Plant Expression Vectors with Unique Cpf1-CO2 Expression Cassettes

Prom_{Mt.Ubiq}::NLS:LbCpf1-CO2:NLS::Term_{Mt}: An expression cassette (SEQ ID NO: 26) for the expression of a Cpf1-CO2 endonuclease was created comprising: a promoter (SEQ ID NO:22), leader (SEQ ID NO:23) and intron (SEQ ID NO:24) derived from Medicago truncatula Ubiquitin operably linked 5′ to the NLS- LbCpf1-CO2-NLS coding sequence (SEQ ID NO: 12) wherein ATGGCG sequence was fused in frame 5′ to SEQ ID NO 12 and served as the translational start site. The resulting sequence was in turn operably linked 5′ to a UTR sequence from a gene from Medicago truncatula (SEQ ID NO:25).

The expression cassette was introduced into an agrobacterium vector that also comprised a gRNA cassette designed to guide LbCpf1 to a unique GmTS1 target site on the soy chromosome. The gRNA comprised a 21 bp crRNA sequence linked to a 23 bp spacer sequence that was complementary to GmTS1 within the soy genome. The gRNA was operably linked to Glycine max U6 Pol III promoter (disclosed in US20170166912, incorporated by reference herein) and a poly T terminator. The vector also comprised an expression cassette for a selectable marker conferring resistance to the antibiotic spectinomycin.

Prom_{EFIa}::NLS:LbCpf1-CO2:NLS::Term_{Mt}: An expression cassette (SEQ ID NO: 31) for the expression of a Cpf1-CO2 endonuclease was created comprising: a promoter (SEQ ID NO:27), leader 5′ (SEQ ID NO:28), intron (SEQ ID NO:29), leader 3′ (SEQ ID NO:30) derived from Cucumis melo EIF1alpha gene operably linked 5′ to the NLS-LbCpf1-CO2-NLS coding sequence (SEQ ID NO: 12) wherein ATGGCG sequence was fused in frame 5′ to SEQ ID NO 12 and served as the translational start site. The resulting sequence was operably linked to a UTR sequence from a gene from Medicago truncatula (SEQ ID NO:25). The expression cassette was introduced into an agrobacterium vector that also comprised a gRNA cassette designed to guide LbCpf1 to a unique GmTS2 target site on the soy chromosome. The gRNA comprised a 21 bp crRNA sequence linked to a 23 bp spacer sequence that was complementary to GmTS2 within the soy genome. The gRNA was operably linked to Glycine max U6 Pol III promoter (disclosed in US20170166912) and a poly T terminator. The vector also comprised an expression cassette for a selectable marker conferring resistance to the antibiotic spectinomycin.

Prom_{At.Ubiq}::NLS:LbCpf1-CO2:NLS::Term_{Gh}: An expression cassette (SEQ ID NO: 36) for the expression of a Cpf1-CO2 endonuclease was created comprising a promoter (SEQ ID NO:32), leader (SEQ ID NO:33) and intron (SEQ ID NO:34) derived from Arabidopsis Ubiquitin 10 gene operably linked 5′ to the NLS-LbCpf1-CO2-NLS coding sequence (SEQ ID NO: 12) where ATGGCG sequence was fused in frame 5′ to SEQ ID NO 12 and served as the translational start site. The resulting sequence was operably linked to a UTR sequence from a gene from Gossypium barbadense (SEQ ID NO: 35).

The expression cassette was introduced into an agrobacterium vector that also comprised a gRNA cassette designed to guide LbCpf1 to a unique GmTS3 target site on the soy chromosome. The gRNA comprised a 21 bp crRNA sequence linked to a 23 bp spacer sequence that was complementary to GmTS3 within the soy genome. The gRNA was operably linked to Glycine max U6 Pol III promoter (disclosed in US20170166912) and a poly T terminator. The vector also comprised an expression cassette for a selectable marker conferring resistance to the antibiotic spectinomycin.

### Example 8

### Testing the Activity of LbCpf1-CO2 Expression Cassettes

The Agrobacterium T-DNA vectors described in Example 7, were introduced into A. tumefaciens. Excised embryos from A3555 Soybean plants were co-cultured with the Agrobacterium containing the vectors by standard methods known in the art and grown on spectinomycin to select for transformed plants. Leaf samples from regenerated plantlets were harvested after 2 weeks and genomic DNA was extracted for Fragment Length Analysis (FLA) to determine genome mutation rates at the target sites GmTS1, GmTS2 and GmTS3 as described in Example 1. A summary of FLA results generated from soy plants stably transformed with the three LbCpf1-CO2 expression cassettes and gRNAs targeting the unique soy genomic target sites is provided in Table 8.

TABLE 8
SUMMARY OF FLA RESULTS GENERATED FROM SOY
PLANTS STABLY TRANSFORMED WITH LBCPF1-CO2
EXPRESSION CASSETTES AND GRNA
TARGETING 3 UNIQUE GENOMIC TARGET SITES

| | Genomic | | # of edited | |
|---|---|---|---|---|
| Expression vector with | target | Plants | plants by | Target site |
| LbCpf1 cassette | site | assayed | FLA | mutation |
| Prom_{Mt.Ubiq}::NLS:LbCpf1- | GmTS1 | 84 | 68 | 81% |
| CO2:NLS::Term_{Mt} | | | | |
| Prom_{EFIa}::NLS:LbCpf1- | GmTS2 | 84 | 58 | 69% |
| CO2:NLS::Term_{Mt} | | | | |
| Prom_{At.Ubiq}::NLS:LbCpf1- | GmTS3 | 84 | 72 | 86% |
| CO2:NLS::Term_{Gb} | | | | |

As shown in Table 8, all three sites targeted for cleavage with the guide/LbCpf1 -CO2 expression systems described above exhibited the presence of mutations which is indicative of DNA cleavage and repair.

### Example 9

### Analysis of LbCpf1(TYC)-CO2 Variant Activity in Corn Plants.

This example describes the testing of a recombinant polynucleotide encoding Lachnospiraceae LbCpf1(TYC) PAM variant nuclease that is optimized for expression in plant cells.

LbCpf1 variants comprising amino acid mutations resulting in altered PAM sequence specificities have been described by Gao et. al. (see Nature Biotech., 2017 August; 35(8):789-792). For example, Gao et. al. have described an LbCpf1(TYC) variant comprising the mutations G532R/K595R that can be engineered to recognize TYCV PAM. Two nucleotide substitutions were introduced into the LbCpf1-CO2 sequence (SEQ ID NO: 10) resulting in LbCpf1(TYC)-CO2 (SEQ ID NO:38) encoding the LbCpf1(TYC) protein (SEQ ID NO:39) comprising the mutations G532R/K595R.

To test the activity of LbCpf1(TYC), an agrobacterium T-DNA vector was generated. The vector comprised a Cpf1 expression cassette (SEQ ID NO:40) comprising the maize ubiquitin promoter (SEQ ID NO: 7) operably linked to a sequence (SEQ ID NO: 41) encoding LbCpf1(TYC)-CO2 comprising two nuclear localization signals (SEQ ID NOs: 42 and 3). The NLS-LbCpf1(TYC)-CO2-NLS was operably linked to a transcription terminator sequence from a rice Lipid transfer protein (LTP) gene (disclosed in US201801058230-0175, incorporated herein by reference). The vector also comprised a gRNA expression cassette encoding gRNAs designed to target two unique target sites in the corn genome, ZmTS13 and ZMTS14. The ZmTS13 and ZMTS14 sites were chosen since the TYCV PAM was present immediately upstream to each site. The 5′PAM for ZmTS13 was the sequence TTCA. The 5′PAM for ZmTS14 was the sequence TCCA. The gRNA expression cassette comprised the Zea mays U6 Pol III promoter (SEQ ID NO: 9) operably linked to two guide RNAs positioned in tandem and targeting the ZmTS13 and ZmTS14 sites. The expression vector also included a third expression cassette containing the selectable marker gene that provides resistance to the herbicide glyphosate.

Corn 01DKD2 cultivar embryos were transformed with the LbCpf1(TYC)-CO2 vector described above by agrobacterium-mediated transformation. Transformed plants were selected on glyphosate, leaf samples from regenerated plantlets were harvested and genomic DNA was extracted for Fragment Length Analysis (FLA) to determine genome mutation rates specifically at ZmTS13 and ZmTS14 sites, as described in Example 1. ZmTS13 and ZmTS14 are arrayed in antisense orientation relative to each other in the genome and overlap by 8 nts, thus individual editing rates at each gRNA target site were not able to be ascertained. Table 9 summarizes the results and shows the cumulative mutation rate detected at or near the two sites in stably transformed corn plants. As shown in Table 9, 48% (40 of the 83) plants tested exhibited the presence of mutations at the expected region which is indicative of DNA cleavage by LbCpf1(TYC) and subsequent repair.

TABLE 9
FLA RESULTS GENERATED FROM CORN PLANTS STABLY
TRANSFORMED WITH LBCPF1(TYC)-CO2 EXPRESSION
CASSETTE AND GRNA TARGETING 2 UNIQUE GENOMIC
TARGET SITES.

| | | | # Edited plants | Cumulative |
|---|---|---|---|---|
| | Target sites | Plants | by FLA | Mutation |
| T-DNA Vector | tested | assayed | analysis | frequency |
| LbCpf1(TYC)- | ZmTS13 | 83 | 40 | 48% |
| CO2 | ZmTS14 | | | |

### Example 10

### Analysis of FnCpf1 Engineered Polynucleotides Optimized for Expression in Plant Cells.

This example describes the design and expression analysis of polynucleotide sequences encoding Francisella novicida (FnCpf1) nuclease that are optimized for expression in plant cells.

A nucleotide sequence of Cpf1 from Francisella novicida (FnCpf1) that was codon optimized for expression in human cells has been described by Zetsche et. al, (Cell 2015, 163, 759-771). To optimize the expression of the FnCpf1 protein (SEQ ID NO:43) in plant cells, the human codon optimized sequence disclosed by Zetsche et. al., (SEQ ID NO:44), described here as FnCpf1-Hs was modified through algorithmic methods, partly based on plant codon frequency tables, to design seven FnCpf1 CO (Codon optimized) sequences (see Table 10).

TABLE 10
CODON OPTIMIZED FNCPF1 AND THE CODON FREQUENCY
TABLES USED TO DESIGN EACH SEQUENCE.

| Codon optimized FnCpf1 | Codon frequency table | SEQ ID NO: |
|---|---|---|
| FnCpf1-CO1 | Glycine max | 45 |
| FnCpf1-CO2 | Monocot | 46 |
| FnCpf1-CO3 | Glycine max | 47 |
| FnCpf1-CO4 | Monocot | 48 |
| FnCpf1-CO5 | Oryza sativa | 49 |
| FnCpf1-CO6 | Oryza sativa | 50 |
| FnCpf1-CO7 | Zea mays | 51 |

### Expression Analysis of FnCpf1-CO Variants Via Quantification of FnCpf1-mOr Intensity:

Three expression cassettes (Prom35S::HIStag:NLS:FnCpf1-CO1:mOrange:NLS::TermNOS; Prom35S::HIStag:NLS:FnCpf1-CO2:mOrange:NLS::TermNOS; and Prom_{35S}::HIStag:NLS:FnCpf1-Hs:mOrange:NLS::Term_{NOS}) were generated by standard cloning techniques and are described below:

(1) Prom_{35S}::HIS tag:NLS:FnCpf1-CO1:mOrange:NLS::Term_{NOS}

The FnCpf1-CO1 coding sequence (SEQ ID NO: 45) was fused 5′ to the coding sequence of mOrange (mOr) from Entacmaea quadricolor. (SEQ ID NO:52) The FnCpf1-CO1:mOrange fusion gene was flanked at the 5′ end with an NLS sequence from potato (SEQ ID NO: 42) and at the 3′ end with an NLS sequence from tomato HSFA1 gene (SEQ ID NO:3) resulting in NLS:FnCpf1-CO1:mOrange:NLS (SEQ ID NO: 53). A nucleotide sequence encoding a HIS tag (MGSS7H) (SEQ ID NO: 54) was introduced at the 5′ end of SEQ ID NO:53. A ‘TAG’ termination codon was introduced to the 3′ end of the resulting nucleotide sequence (SEQ ID NO: 55) which was then operably linked to the Cauliflower mosaic virus 35S promoter (disclosed in US9938535-0047, incorporated herein by reference) and an Agrobacterium NOS terminator (MK078637). The expression cassette (SEQ ID NO: 56) was cloned into a plant expression vector.

(2) Prom_{35S}::HIS tag:NLS:FnCpf1-CO2:mOrange:NLS::Term_{NOS}

The FnCpf1-CO2 coding sequence (SEQ ID NO: 46) was fused 5′ to the coding sequence of mOrange (mOr) from Entacmaea quadricolor. (SEQ ID NO:52). The FnCpf1-CO2:mOrange fusion gene was flanked at the 5′ end with an NLS sequence from potato (SEQ ID NO: 42) and at the 3′ end with an NLS sequence from tomato HSFA1 gene (SEQ ID NO:3) resulting in NLS-FnCpf1-CO2-NLS(SEQ ID NO:57). A nucleotide sequence encoding a HIS tag (MGSS7H) (SEQ ID NO: 54) was introduced at the 5′ end of SEQ ID NO:57. A ‘TAG’ termination codon was introduced to the 3′ end of the resulting nucleotide sequence(SEQ ID NO:58) which was then operably linked to the Cauliflower mosaic virus 35S promoter and an Agrobacterium NOS terminator (MK078637). The expression cassette (SEQ ID NO:59) was cloned into a plant expression vector.

(3) Prom_{35S}::HIS tag:NLS:FnCpf1-Hs:mOrange:NLS::Term_{NOS}

The human codon-optimized Cpf1 (FnCpf1-Hs) nucleotide sequence described by Zetsche et. al. (Cell 2015, 163, 759-771) (SEQ ID NO:44) was fused 5′ to the coding sequence of mOrange (mOr) from Entacmaea quadricolor. (SEQ ID NO:52). The FnCpf1-Hs:mOrange fusion gene was then flanked at the 5′ end with an NLS sequence from potato (SEQ ID NO: 42) and at the 3′ end with an NLS sequence from tomato HSFA1 gene (SEQ ID NO:3) resulting in NLS-FnCpf1-Hs:mOrange-NLS(SEQ ID NO: 60). A nucleotide sequence encoding a HIS tag (MGSS7H) (SEQ ID NO: 54) was introduced at the 5′ end of SEQ ID NO:60. A ‘TAG’ termination codon was introduced to the 3′ end of the resulting nucleotide sequence (SEQ ID NO:61) which was then operably linked to the Cauliflower mosaic virus 35S promoter and an Agrobacterium NOS terminator (MK078637). The expression cassette (SEQ ID NO:62) was cloned into a plant expression vector.

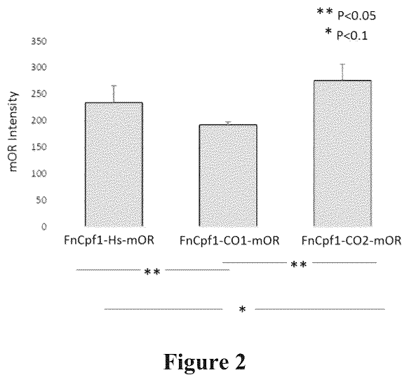

To evaluate and quantify the expression of the fusion proteins, corn leaf protoplasts were transfected with expression vectors comprising either of the three expression cassettes described above. Since mOrange was fused to FnCpf1-CO1, FnCpf1-CO2 and FnCpf1-Hs, the relative mOrange fluorescence levels reflects FnCpf1 expression levels. Transformations were carried out using standard polyethylene glycol (PEG) based transfection methods. To quantify transformation frequency, an expression vector comprising the luciferase gene was co-transfected. Following transformation, the protoplasts were incubated in the dark in incubation buffer and harvested after 48 hours. Transformation efficiency was calculated by quantifying luciferase expression. The average mOrange expression from 5 technical replicates was determined using Operetta™ (Perkin Elmer) analysis software. As shown in FIG. 2, mOrange fluorescence was detected from all three samples. The observed intensity was the highest in protoplasts expressing the FnCpf1-CO2-mOrange expression construct.

### Expression Analysis FnCpf1-CO Variants Via Qualitative Western Blots:

In addition to the three expression constructs described above, five expression constructs were generated and are described below:

(4) Prom_{Ubiq}::NLS:FnCpf1-CO3:NLS::Term_{Os}:

The FnCpf1-CO3 sequence (SEQ ID NO:47) was flanked at the 5′ end with an NLS sequence from potato (SEQ ID NO: 42) and at the 3′ end with an NLS sequence from tomato HSFA1 gene (SEQ ID NO:3) resulting in NLS-FnCpf1-CO3-NLS (SEQ ID NO:63). An ATG sequence encoding the translation initiation codon was added 5′ to the potato NLS sequence and a TGA termination codon sequence was introduced 3′ to the tomato NLS sequence. The resulting sequence was operably linked to the maize ubiquitin promoter (SEQ ID NO: 7) and a transcription terminator sequence from a rice (SEQ ID NO:64). The FnCpf1-CO3 expression cassette sequence is set forth as SEQ ID NO:65. The expression cassette was cloned into a plant expression vector.

(5) Prom_{Ubiq}::NLS:FnCpf1-CO4:NLS::Term_{Os}:

The FnCpf1-CO4 sequence (SEQ ID NO:48) was flanked at the 5′ end with an NLS sequence from potato (SEQ ID NO: 42) and at the 3′ end with an NLS sequence from tomato HSFA1 gene (SEQ ID NO:3) resulting in NLS-FnCpf1-CO4-NLS(SEQ ID NO:66). An ATG sequence encoding the translation initiation codon was added 5′ to the potato NLS sequence and a TAG termination codon sequence was introduced 3′ to the tomato NLS sequence. The resulting sequence was operably linked to the maize ubiquitin promoter (SEQ ID NO: 7) and a transcription terminator sequence from a rice (SEQ ID NO: 64). The FnCpf1-CO4 expression cassette sequence is set forth as SEQ ID NO:67. The expression cassette was cloned into a plant expression vector.

(6) Prom_{Ubiq}::NLS:FnCpf1-CO5:NLS::Term_{Os:}

The FnCpf1-CO5 sequence (SEQ ID NO:49) was flanked at the 5′ end with an NLS sequence from potato (SEQ ID NO: 42) and at the 3′ end with an NLS sequence from tomato HSFA1 gene (SEQ ID NO:3) resulting in NLS-FnCpf1-CO5-NLS (SEQ ID NO:68). An ATG sequence encoding the translation initiation codon was added 5′ to the potato NLS sequence and a TGA termination codon sequence was introduced 3′ to the tomato NLS sequence. The resulting sequence was operably linked to the maize ubiquitin promoter (SEQ ID NO: 7) and a transcription terminator sequence from a rice (SEQ ID NO: 64). The FnCpf1-CO5 expression cassette sequence is set forth as SEQ ID NO:69. The expression cassette was cloned into a plant expression vector.

(7) Prom_{Ubiq}::NLS:FnCpf1-CO6:NLS::Term_{Os}:

The FnCpf1-CO6 sequence (SEQ ID NO:50) was flanked at the 5′ end with an NLS sequence from potato (SEQ ID NO: 42) and at the 3′ end with an NLS sequence from tomato HSFA1 gene (SEQ ID NO:3) resulting in NLS-FnCpf1-CO6-NLS (SEQ ID NO:70). An ATG sequence encoding the translation initiation codon was added 5′ to the potato NLS sequence and a TGA termination codon sequence was introduced 3′ to the tomato NLS sequence. The resulting sequence was operably linked to the maize ubiquitin promoter (SEQ ID NO: 7) and a transcription terminator sequence from a rice (SEQ ID NO: 64). The FnCpf1-CO6 expression cassette sequence is set forth as SEQ ID NO:71. The expression cassette was cloned into a plant expression vector.

(8) Prom_{Ubiq}::NLS:FnCpf1-CO7:NLS::Term_{Os}:

The FnCpf1-CO7 sequence (SEQ ID NO:51) was flanked at the 5′ end with an NLS sequence from potato (SEQ ID NO: 42) and at the 3′ end with an NLS sequence from tomato HSFA1 gene (SEQ ID NO:3) resulting in NLS-FnCpf1-CO7-NLS (SEQ ID NO:72). An ATG sequence encoding the translation initiation codon was added 5′ to the potato NLS sequence and a TGA termination codon sequence was introduced 3′ to the tomato NLS sequence. The resulting sequence was operably linked to the maize ubiquitin promoter (SEQ ID NO: 7) and a transcription terminator sequence from a rice (SEQ ID NO: 64). The FnCpf1-CO7 expression cassette sequence is set forth as SEQ ID NO:73. The expression cassette was cloned into a plant expression vector.

Corn protoplast cells were transformed with the eight plant expression vectors described above and in Table 11. As a negative control, cells were transformed with an expression vector for GFP. Transformations were carried out using standard polyethylene glycol (PEG) based transfection methods. Following transformation, the protoplasts were incubated in the dark in incubation buffer and harvested after 48 hours. 32*10⁴ cells from each transformation were lysed using 50 uL of lysis buffer. Total protein was extracted from each of the lysed samples and 30 ug protein per sample was resolved on an SDS-PAGE gel and electro-blotted onto nitrocellulose membranes by standard methods. 5 ng, 1 ng and 500 pg of purified FnCpf1 protein were loaded as positive controls. Western blots using anti-FnCpf1 antibody (Cell Signaling Technology, Danvers, MA) were performed to detect the presence of FnCpf1 proteins using standard methods. As noted in Table 11, a band corresponding to the FnCpf1-mOr was visually observed in the lanes containing protein extract from protoplasts expressing FnCpf1-CO2-mOr (Sample 3). Similarly, bands corresponding to FnCpf1 were visually observed in the lanes containing protein extract from protoplasts expressing FnCpf1 -CO3 and FnCpf1-CO4 (Samples 4 and 5).

TABLE 11
EXPRESSION ANALYSIS FNCPF1-CODON OPTIMIZED VARIANTS VIA
QUALITATIVE WESTERN BLOTS

| | | Protein band |
|---|---|---|
| Sample | Expression cassette | observed |
| 1 | Prom_{35S}::HIStag:NLS:FnCpf1Hs:mOrange:NLS::Term_{NOS} | No |
| 2 | Prom_{35S}:: HIS tag:NLS:FnCpf1-CO1:mOrange:NLS::Term_{NOS} | No |
| 3 | Prom_{35S}:: HIS tag:NLS:FnCpf1-CO2:mOrange:NLS::Term_{NOS} | Yes |
| 4 | Prom_{Ubiq}::NLS:FnCpf1-CO3:NLS::Term_{Os} | Yes |
| 5 | Prom_{Ubiq}::NLS:FnCpf1-CO4:NLS::Term_{Os} | Yes |
| 6 | Prom_{Ubiq}::NLS:FnCpf1-CO5:NLS::Term_{Os} | No |
| 7 | Prom_{Ubiq}::NLS:FnCpf1-CO6:NLS::Term_{Os} | No |
| 8 | Prom_{Ubiq}::NLS:FnCpf1-CO7:NLS::Term_{Os} | No |
| 9 | 5 ng purified FnCpf1 protein (Positive control) | Yes |
| 10 | 1 ng purified FnCpf1 protein (Positive control) | Yes |
| 11 | 500 pg purified FnCpf1 protein (Positive control) | Yes |
| 12 | Prom_{35S}::GFP::Term_{NOS} (Negative control) | No |

### Example 11

### Analysis of FnCpf1 Activity in Corn Protoplasts.

The assay used to evaluate FnCpf1 activity in corn protoplasts was integration of a blunt-end, double-stranded DNA (dsDNA) fragment into the DSB (Double stranded break) created by FnCpf1 protein at a specific target site.

The blunt-end dsDNA fragment (disclosed in WO2019084148-021, incorporated herein by reference) was prepared by pre-annealing complementary ssDNA oligonucleotides. The ZmTS9 target site was chosen as the insertion site and a gRNA expression cassette targeting TS9 was designed. The expression cassette comprised a synthetic U6 promoter operably linked to a 21 bp crRNA sequence linked to a 23 bp spacer sequence that was complementary to ZmTS9 in the corn genome. The gRNA expression cassette was introduced into a plant expression vector. The gRNA vector and the eight plant vectors described in Example 11, each containing an expression cassette for a codon optimized FnCpf1 variant were co-transformed into isolated corn leaf protoplasts along with the double-stranded DNA (dsDNA) fragment essentially as described in patent application publication WO2015131101 (incorporated herein by reference), with minor modifications. Approximately 3.2×10⁵ protoplasts were transformed using PEG with a total of 12 μg of plasmid DNA and 50 pmoles of the dsDNA fragment (assays 2-9 in Table 12). Protoplast samples lacking the nuclease expressing plasmids served as a negative control (see assay 10 in Table 12). Additionally, protoplast samples transformed with nuclease vectors and gRNA cassettes lacking the spacer sequence were used as negative controls (see assays 11-19 in Table 12). As a positive control (assay 1 in Table 12), protoplasts were transformed with the gRNA cassette and a vector comprising an expression cassette (SEQ ID NO:74) for LbCpf1-CO2 that has been shown to be active in corn (see Examples 3-4). The expression cassette (SEQ ID NO: 20) comprised NLS-LbCpf1-CO2-NLS (SEQ ID NO:12) with ATGGCG fused in frame 5′ to SEQ ID NO 12 as the translational start site, and TGA termination codon fused 3′ to SEQ ID NO:12. The resulting sequence was operably linked to the maize ubiquitin promoter (SEQ ID NO: 7) and a transcription terminator sequence from a rice (SEQ ID NO:64). To determine transformation efficiency, 3 ug of GFP internal control plasmid was transformed along with test constructs. Following transformation, the corn protoplasts were incubated in the dark and harvested after 48 hours. Genomic DNA was extracted and assayed for integration of the dsDNA fragment. Integration of the dsDNA fragment into the genomic DNA was detected by standard PCR and agarose gel electrophoresis to assess PCR amplicons. The dsDNA fragment may have integrated in either a 5′ or 3′ orientation with respect to the 5′- and 3′-ends of the DSB. Therefore, two PCR primer sets were run for the target site where the primer sets contained a primer specific to the dsDNA fragment and a primer specific to either the 5′ side or the 3′ side of the DSB at TS11. The PCR amplicons were separated using standard agarose gel electrophoresis and the size of the amplicon was confirmed by comparison to a molecular weight marker. The presence of a band of expected size was indicative of site-directed integration of the donor oligo at the ZmTS9 site following FnCpf1 mediated dsDNA cleavage. As shown in Table 12, expected bands were amplified from protoplasts expressing LbCpf1-CO2, FnCpf1-CO1, FnCpf1-CO2, FnCpf1-CO3, FnCpf1-CO4, FnCpf1-CO6, FnCpf1-CO7 along with the cognate gRNA cassette and ds DNA. Expected bands were not amplified from protoplasts expressing FnCpf1-CO5 or any of the negative controls.

TABLE 12
FNCPF1 MEDIATED SITE DIRECTED INTEGRATION OF DSDNA OLIGO AT ZMTS9
TARGET SITE.

| | | gRNA | Expected |
|---|---|---|---|
| | | targeting | band |
| Assay | Nuclease Expression cassette | ZmTS9 | amplified |
| 1 | Prom_{Ubiq}::NLS:LbCpf1-CO2:NLS::Term_{Os} (Positive control) | + | Yes |
| 2 | Prom_{35S}::HIStag:NLS:FnCpf1Hs:mOrange:NLS::Term_{NOS} | + | No |
| 3 | Prom_{35S}:: HIS tag:NLS:FnCpf1-CO1:mOrange:NLS::Term_{NOS} | + | Yes |
| 4 | Prom_{35S}:: HIS tag:NLS:FnCpf1-CO2:mOrange:NLS::Term_{NOS} | + | Yes |
| 5 | Prom_{Ubiq}::NLS:FnCpf1-CO3:NLS::Term_{Os} | + | Yes |
| 6 | Prom_{Ubiq}::NLS:FnCpf1-CO4:NLS::Term_{Os} | + | Yes |
| 7 | Prom_{Ubiq}::NLS:FnCpf1-CO5:NLS::Term_{Os} | + | No |
| 8 | Prom_{Ubiq}::NLS:FnCpf1-CO6:NLS::Term_{Os} | + | Yes |
| 9 | Prom_{Ubiq}::NLS:FnCpf1-CO7:NLS::Term_{Os} | + | Yes |
| 10 | None | + | No |
| 11 | Prom_{Ubiq}::NLS:LbCpf1-CO2:NLS::Term_{Os} | − | No |
| 12 | Prom_{35S}::HIStag:NLS:FnCpf1Hs:mOrange:NLS::Term_{NOS} | − | No |
| 13 | Prom_{35S}:: HIS tag:NLS:FnCpf1-CO1:mOrange:NLS::Term_{NOS} | − | No |
| 14 | Prom_{35S}:: HIS tag:NLS:FnCpf1-CO2:mOrange:NLS::Term_{NOS} | − | No |
| 15 | Prom_{Ubiq}::NLS:FnCpf1-CO3:NLS::Term_{Os} | − | No |
| 16 | Prom_{Ubiq}::NLS:FnCpf1-CO4:NLS::Term_{Os} | − | No |
| 17 | Prom_{Ubiq}::NLS:FnCpf1-CO5:NLS::Term_{Os} | − | No |
| 18 | Prom_{Ubiq}::NLS:FnCpf1-CO6:NLS::Term_{Os} | − | No |
| 19 | Prom_{Ubiq}::NLS:FnCpf1-CO7:NLS::Term_{Os} | − | No |

1. A recombinant nucleic acid comprising the sequence of SEQ ID No 75.

2. The recombinant nucleic acid of claim 1, further comprising one or more components selected from the group consisting of: a nucleic acid sequence encoding one or more nuclear localization signals, an operably linked promoter, one or more introns, one or more kozak sequences, one or more leader sequences, and one or more terminator sequences.

3. (canceled)

4. The recombinant nucleic acid of claim 2, wherein the promoter comprises a sequence selected from the group consisting of SEQ ID Nos: 7, 22, 27, and 32.

5. (canceled)

6. (canceled)

7. A plant cell comprising the recombinant nucleic acid of claim 1.

8.-14. (canceled)

15. A composition comprising: (a) the recombinant nucleic acid of claim 1, and (b) a recombinant nucleic acid encoding a guide RNA comprised of at least one crRNA and at least one spacer RNA sequence.

16. The composition of claim 15, wherein the composition is provided on a particle suitable for biolistic delivery to a plant cell.

17. A method for modifying a target sequence in the genome of a plant cell, comprising:
a) introducing into the plant cell the recombinant nucleic acid of claim 1 operably linked to a promoter, and
b) introducing into the plant cell a guide polynucleotide comprising a nucleic acid sequence that is complementary to a target sequence or a recombinant nucleic acid encoding the guide polynucleotide, wherein the guide polynucleotide and a Cpf1 nuclease expressed from the recombinant nucleic acid form a complex that can bind to and modify the target sequence.

18. The method of claim 17, wherein the promoter comprises a sequence selected from the group consisting of SEQ ID Nos: 7, 22, 27, and 32.

19. (canceled)

20. (canceled)

21. The method of claim 17, wherein the plant cell is a maize, cotton, soybean, canola, wheat, tomato, rice, brassica, melon, cucurbit, or lettuce cell.

22. The method of claim 17, further comprising introducing a donor DNA to the plant cell.

23. The method of claim 22, further comprising identifying at least one plant cell comprising in its genome the donor DNA, or a portion thereof, integrated into or near said target sequence.

24. A method for modifying a target sequence in the genome of a plant cell, the method comprising: introducing a guide polynucleotide comprising a nucleic acid sequence that is substantially complementary to the target sequence, or a recombinant nucleic acid encoding the guide polynucleotide, into a plant cell comprising in its genome the recombinant nucleic acid of claim 1 operably linked to a promoter, wherein the guide polynucleotide and a Cpf1 nuclease expressed from the recombinant nucleic acid form a complex that can bind to and modify the target sequence.

25. The method of claim 24, wherein the promoter comprises a sequence selected from the group consisting of SEQ ID Nos: 7, 22, 27, and 32.

26. (canceled)

27. (canceled)

28. The method of claim 24, wherein the plant cell is a maize, cotton, soybean, canola, wheat, tomato, rice, brassica, melon, cucurbit, or lettuce cell.

29. The method of claim 24, further comprising introducing a donor DNA to the plant cell.

30. The method of claim 29, further comprising identifying at least one plant cell comprising in its genome the donor DNA, or a portion thereof, integrated into or near said target sequence.

31. A kit for modifying a target sequence in the genome of a plant cell, the kit comprising a guide polynucleotide comprising a nucleic acid sequence that is complementary to a target sequence or a recombinant nucleic acid encoding the guide polynucleotide, and the recombinant nucleic acid of claim 1.

32. (canceled)

33. The kit of claim 31, further comprising a recombinant nucleic acid encoding a selectable marker.
